SEQUENCE LISTING (157 sequences)

SEQ ID NO: 1 - DNA, 1006 bp, Mus musculus
cagctgcagtccaggctcatttaggcagtgagggagggtaagcaagcatcacaaattatcaagctagctagtggaagattagtagtagctgggcagaactaaggcaggattgggagaaggtagggggattttcagcctgggcaaaattcctatcttagaatgactattctatgcacagaactaggaatacagatagattggctaaggggagaaatctgtgtacaaacagtgtccattatgaactctaccccagcaaaagtgaaaggaattattgagaacaaaggttgctgctaaactggacaacacattgaacttggggatttccaacccagctactcacatataagcagacacaaggatcttttcttattcagcctctgactacagacatggcttctgtccgcacctgtgttgtccgttcagaccaatggcggttaatgacctttgagaccactgagaatgacaaagtgaagaagataaatgaacatattaggtcccaaaccaaggtctctgtacaggaccagatccttctgctagactccaaaatcctcaagccccatcgaaaattgtcatcctatgggattgacaaggaaaccactatccaccttaccctgaaggtggtgaagcccagtgatgaagagctgcccttgtttctggtggagtccaaaaacgaggggcaaaggcacctcctccgagttcgaagatccagctcagtggcccaggtgaaagagatgatcgagagtgtgacctctgtgatccctaagaagcaggttgtgaattgcaacggaaagaagctggaagatggaaagatcatggctgactacaacatcaagagtggcagtttgctctttctgacaacacactgcactgggggatgaccacggggatgcggtgatgagaagctcaaagccgatttcctctgagctattaccaatcacatctcttgatgatataaaaaattaacgagaatgaaataaatatttgagggaagtggagtagattaaaa

SEQ ID NO: 2 - protein, 162 aa, Mus musculus
MASVRTCVVRSDQWRLMTFETTENDKVKKINEHIRSQTKVSVQDQILLLDSKILKPHRKLSSYGIDKETTIHLTLKVVKPSDEELPLFLVESKNEGQRHLLRVRRSSSVAQVKEMIESVTSVIPKKQVVNCNGKKLEDGKIMADYNIKSGSLLFLTTHCTGG

SEQ ID NO: 3 - DNA, 1640 bp, Mus musculus
gggcactttctggccagaatccgcggggccccatgctgtggtcccaggaggcacgcgggtcctggctgcagcagcgggactgcagggcgagcctccctggaacgggtgaagttgattccaagtactcagctatggaagatgcaacctaaaacgagagacctgctgcgctcctcttggaatttgtagcctcacattcaatgccaactttctttggaaggagacttgggagggtctgagagccatgaggatggtgctgaaaagactcgattccgttctggaattctgccgccaggcggcgctgctttgaagagccaggcagctacctgctggagcccgaccttccaccagggggcggaggtctccagagaggccctccgggccccgctggcctcggagccaagtcttaaagaggagcctggacccaagcttctcctcagcgactgatgatgttaaactatacgcttctgtgattggtttagccgtggtaagacgggctgctattggagatgctctgttgaaacgtgatggatctggaagggagctgggcacccgacactcgagtggtagctcccatttactgtgggtgttaaggtatggagcatgagcagaagtcaggagctgtgctgctgaggctgctgcgccttctgtggctgctgccccactcctgggcggtgctcgaagcatctacaccagtgttctgggatgacccacagaaccacacattccggcacactctattctgccaggacgggattcccaacatagggctctcggagacctatgacgaggacgaactcttctccttcgacttctcccagaacaccagagtgccccgactgcctgactttgcagagtgggctcagggacagggggatgcctctgccattgcatttgacaaaagcttctgcgagatgttgatgcgggaagtgagcccgaagcttgaaggtcaaatcccagtgtccagaggtttgcctgtggcagaggtattcacactgaagcccctggagtttggcaagcccaacacgttggtctgtttcatcagcaacctctttccaccgaccctgaccgtgaactggcagcttcactcggcccctgtggagggagccagccccacatccatctcagctgtcgatgggctgaccttccaggctttctcttatttaaacttcacaccggaaccctttgacctttactcctgcactgtgacacacgagattgaccgctacacggcaattgcctattgggtaccccagaacgccctgccttcggatctcctagagaatgccctgtgtggtgtggcctttggcctcggtgtgctaggcaccatcatgggcattgtcttcttcctctgctcccagagaccgtgctcaggtgactgattcttctcaaggagatcttggaacagcacaggccaggccagcggggatgtccaggcatctcgtgactacccaaggtctctcatcggagccgaagtcccagggatccctcagggtgtgtgccagcgtgcaggtgtctcagctgagggttccgctgtccctggacttgcatccagaagagccttcatcaggagccccaggctccacctaaccactaccttataccttccctcttccaggaaataaacttcatttcttaatgcc

SEQ ID NO: 4 - protein, 261 aa, Mus musculus
MEHEQKSGAVLLRLLRLLWLLPHSWAVLEASTPVFWDDPQNHTFRHTLFCQDGIPNIGLSETYDEDELFSFDFSQNTRVPRLPDFAEWAQGQGDASAIAFDKSFCEMLMREVSPKLEGQIPVSRGLPVAEVFTLKPLEFGKPNTLVCFISNLFPPTLTVNWQLHSAPVEGASPTSISAVDGLTFQAFSYLNFTPEPFDLYSCTVTHEIDRYTAIAYWVPQNALPSDLLENALCGVAFGLGVLGTIMGIVFFLCSQRPCSGD

SEQ ID NO: 5 - DNA, 2030 bp, Mus musculus
agcagaccaggaaggcagcagccctgaccaccaggagccagatatcacagacactggcagctggctcagctctgcaggagaagagagtaatggctggtgtctcccctgctgtctttggctgcccagatgtcaccctgggaaggaacactgcagtccgtgaagtacaagaaaatgtcacatcagtggacagtttaacactggtctccagcaacactgactttgccttcagcctctacaggaagctggttttgaagaatccagatgaaaatgttgtcttctccccattcagcatctgcactgccttggccctcctgtccctgggagcaaagagcaacaccctgaaggaaatcctagaaggtctcaagttcaacctcacagagacccctgaaccagacatccaccagggctttaggtacttgctagaccttctaagtcagccagggaaccaggtacagatcagcacaggcagtgccctgtttattgaaaagcacctacagatcctggcagagttcaaggagaaggcaagggctctgtaccaggctgaggccttcacagcagatttccagcaacctctcaaggccacaaagctcatcaatgactatgtgagcaatcacacccaggggaagatcaaggaactcatctcaggcctgaaagagagcacattgatggtgctggtgaactacatctactttaaaggcaaatggaagaacccctttgacccgaatgatacatttaagtccgagttctacttggatgagaagaggtctgtgattgtgtccatgatgaaaactggttacctgacaactccctacttccgggatgaggagctgtcctgcactgtggtggagctgaaatacacaggaaatgccagtgccatgttcatcctccctgaccagggcaggatgcagcaggtggaagcaagcttacaaccagagaccctgaggaagtggaagaactctctgaagcccaggatgatacatgagctccgcctgcccaagttctccatctccaccgactacagcctggagcacatccttcctgagttgggcatcagggaagtcttctccacacaggctgacctgtctgcaatcacaggaaccaaggatctgagagtctctcaggtggtccacaaggctgtgctggatgtggctgagacaggcacagaggctgctgctgccacaggaatggcaggtgtcggatgttgtgcagtttttgactttctggaaatatttttcaacaggccattcctgatgattatctctgacacaaaagctcacattgccctctttatggcaaaagttacaaatccagagagatctatgaacttcccaaatggtgagggtgcttcttcccaacggctggaatctaagcgtttatgttttggtgatccgctatgcctgattggacagtgacacttaccaactctgtcacgtcctcatgcagagaaagaagcctatggctggttattgtcagactctctgtcatgatggtatcacgaaatcaggttcctgacctgaaatttgtgttattccctttccctgctgtctccattgtatctgcctcaacccaaaagcctgggcccagcagtgaggctctctcctaacttaggctctgcttatgtctgccttcatcctttcagtcatgatgggactgtgcaagtttacaggccaacccataaggactaagaaaggaacctggtgccctgggtctatctgcagcatctgaaatgcttggtgcccagatctgccttatgcttgccttcctctgggcagaattatcctcagcccctgcatagtctcctggccccacccagatctggcacaggtggagatctcagccctgcagctgcatggggcctgtgggtcagagcagctcctccccctgtagcactcctactcagaacagagtagtctttctcaatgtctcttgttgaccaactacgtaggactggtggacacctctgcattcatcacacaaattgaaagggcaggagctaaggatcaataaacatgtaactgcaaaaaaaaaaa

SEQ ID NO: 6 - protein, 440 aa, Mus musculus
MAGVSPAVFGCPDVTLGRNTAVREVQENVTSVDSLTLVSSNTDFAFSLYRKLVLKNPDENVVFSPFSICTALALLSLGAKSNTLKEILEGLKFNLTETPEPDIHQGFRYLLDLLSQPGNQVQISTGSALFIEKHLQILAEFKEKARALYQAEAFTADFQQPLKATKLINDYVSNHTQGKIKELISGLKESTLMVLVNYIYFKGKWKNPFDPNDTFKSEFYLDEKRSVIVSMMKTGYLTTPYFRDEELSCTVVELKYTGNASAMFILPDQGRMQQVEASLQPETLRKWKNSLKPRMIHELRLPKFSISTDYSLEHILPELGIREVFSTQADLSAITGTKDLRVSQVVHKAVLDVAETGTEAAAATGMAGVGCCAVFDFLEIFFNRPFLMIISDTKAHIALFMAKVTNPERSMNFPNGEGASSQRLESKRLCFGDPLCLIGQ

SEQ ID NO: 7 - DNA, 3348 bp, Mus musculus
gcagaaaagagtcctcttcaacaagggtgttcctgagagcatctgggaatcagaagagccaaggacacctcagagctctggatctgggcttttcccctgttcatggctgcatcgcctctcctcagtttgtgagcctctccctgtagagcagccatggccttgaagaatgtcccgtttcgctcagaggtcctagcctggaactcagacaacctcgctgactatttcaggaagctgaactacagggactgtgagaaggctgtgaagaaataccacattgatggtgctcggtttttgaacctgacagaaaatgacattcagaagttccctaagctgaggatgccacttctcagtaagttgagtcaagatatcaacaagaacgaagagagaaggagcattttcacacgcaaaccccagattccacggtttcttgaggagacagaaagccacgaagaagacgatggtggctggtcatcctttgaagatgactatgaaagtcccaatgatgatgatccagatggggaggacgatggtgactatgagtcccctaatgaggaggaacaggccctggtggacgatgctgctgactatgagcctccaccctccaacaacgaggaggctctgcagagctccatcctgccccccaactctttccacaacaccaactccatgtacatcgaccgacctcccactggcaaggtctcccagcagcctccagtgcccccactgagaccaaaacctgccctccctcccctgccaacaggcaggaatcactcgccactgtctccaccccatcccaaccacgaagagcccagcagaagtggaaacaacaagacagcaaagttgcctgctccatcaatagacagaagcacaaaacctcccctggaccgctcactggcccctcttgaccgagagcccttcatactaggaaagaaaccaccattttctgataagccctcagccccattggggagggaacatttacccaagatacaaaagcctcctttgccaccagccatggacagacacgaaagaaatgaaagactcggacccgtgacaacgaggaagccaccagttccaaggcatggaaggggaccagacagaagagagaatgatgaagatgatgtgcatcagagacctttgccacagccgtcactaccttccatgagctccaacacattcccttcgagatctgtccagcccagctccaagaacacattcccattggcccacatgccgggagccttctcagaaagcaacattggctttcagcagagtgcctccctgccgccatacttctctcaaggtccaggcaacaggccacctctcagaagtgaaggcagaaacttacccttgccggttccaaacaggcctcagcctccatctcctggggaagaagagacacccttagatgaagagtggtatgtctcttatatcacccgaccagaggcagaagctgctctgaggaagataaaccaggatggcaccttcctggttagagacagctctaagaagacagcgaacaatccgtatgtcctcatggtgctgtacaaagacaaagtttacaacatccaaatccgctaccaggaggaaagtcaagtctacttgttgggcactggacttcgaggaaaagaggacttcctgtctgtatcagatattattgactacttcaggaaaatgccgctgttgctcattgatggaaagaatcgagggtccagataccagtgcacactaacacacgctgcaggctgtctgtagcaagttagccaagaaatgagcctttctgacttggtggtcactgcggcgaaatcacgtgaccttagcaaatcgccgagcacttaggagtagactgttaactcgggcccagagttctgccctgcccttttaaagtatctgaagattgttgaataccacatgatttatttattttctttgatgtttatagagtatagaagtgatcatgttcacatttgaagtctaagagtgcatggcagacatgagattttgaacatccatcccaaaatacagtacttgatacagtacagaaataatgtggagcaagctgaaaaagtactgaaactggtcatttttgatatattttataatgaatcttttgtcaggccgaggcatgttctgttttttacacaacttaaaatgattaaaattcattttggagcagcaataacttaagaggctccaaaaaaaaaaaatagaactccatcgtttcttatttggttttacagggtgtgtttttatgtgttccctttcgtaatgagattaatgcatgtggggataacttcctgggattaaagttatcgtttgctttacttttttcgatccttaagacaacgttgtttggtccttgctgttatatttttattttaaagcaaatgtaagctacctttctgaacatacaagaagaaaataggcttaggttcagtgatgcagctttgctgtcaaacatgctgggcttcttgttgcttctcttatcacagaccctcatgtctccatccatggtggcctccgttctagtactgaaggtctcctgggaagacgagcaatgcttggtaaacacacacacacgcacacacacacacacacacacacacacacacacacttcgagcctaccttcctactagatgatacttgcattcgctggccttcagttagacagtttgactttctgggatggtcgcctgttcctgttcctttgggcctgcaacagttcatagaggatcatgaagtgagaatgtgaggacatccgttcacttcatggtgactgggaaacaaaagaaaggacaagaagcgcatttccctccatgtcttgcatcttctgcctaactgggcttcatctcctaagattccaccactgcccaatgctgccctggtcgggggcagggggccaagcttctaccatgtgggcctttggagaattctatccaaacctctgctctggttttctgctttattagcccaacaagtatgttgcaaaactgctatcttgtcctcagattggcttttaaaccctttgaagcacctcagatctccttcagttttgaaatcacacttaacaaaaggtagagagagatttctggcctatgttggtttaacttgccatttttgactttatgatggcatggaagtgatgcttgttaagcacgaactgtacgtcaaagtttgaattttgcttttctggggctagcagcagatgctatgaaatgtaagagtcttggctagcaggtgatcaccagggcacccatcagtactttacatgttttatgttgaagctgatttttttctttttgtggaggggctgctatgaacgctcaataaattaaataatgcattc

SEQ ID NO: 8 - protein, 533 aa, Mus musculus
MALKNVPFRSEVLAWNSDNLADYFRKLNYRDCEKAVKKYHIDGARFLNLTENDIQKFPKLRMPLLSKLSQDINKNEERRSIFTRKPQIPRFLEETESHEEDDGGWSSFEDDYESPNDDDPDGEDDGDYESPNEEEQALVDDAADYEPPPSNNEEALQSSILPPNSFHNTNSMYIDRPPTGKVSQQPPVPPLRPKPALPPLPTGRNHSPLSPPHPNHEEPSRSGNNKTAKLPAPSIDRSTKPPLDRSLAPLDREPFILGKKPPFSDKPSAPLGREHLPKIQKPPLPPAMDRHERNERLGPVTTRKPPVPRHGRGPDRRENDEDDVHQRPLPQPSLPSMSSNTFPSRSVQPSSKNTFPLAHMPGAFSESNIGFQQSASLPPYFSQGPGNRPPLRSEGRNLPLPVPNRPQPPSPGEEETPLDEEWYVSYITRPEAEAALRKINQDGTFLVRDSSKKTANNPYVLMVLYKDKVYNIQIRYQEESQVYLLGTGLRGKEDFLSVSDIIDYFRKMPLLLIDGKNRGSRYQCTLTHAAGCL

SEQ ID NO: 9 - DNA, 1256 bp, Mus musculus
gattggatctcaggaggggctgagtgacttggcgccacataggcagcaaggagcaaggggcctgagcaagaggtaacatatttacctcagtgtgtgaagatcatttgcacaggctggggcaggagagaacctgagacccctgcaggaggtgcccaggggcctggccttcctgcaccttcagagagtggaacatggctgcactctggctgctgctgctggtcctcagtctgcactgtatgggggcaggtggctttgtggctcatgtggaaagcacgtgcgtgctggatgatgctggaaccccacaggacttcacatactgtgtttccttcaacaaagatctgctggcctgctgggatccaatagtgggaaaaatagtcccctgtgaatttggggtgctgtatccattggccgaaaatttttcaaggatcctcaacaaagaagagagccttctccagcgtttgcaaaacgggcttctggactgtgcctcccacacccagcccttctggaatgcgctgacccacagaacgagaccgccatctgtccgagtagcccaaaccacaccttttaacacaagggagcccgtgatgctggcctgctacgtctggggcttctatccagcggatgtgaccatcacatggatgaagaatgggcagcttgtcccttcccacagcaacaaggagaagacggctcagcccaatggagactggacataccagacagtctcctacctagccctaaccccttcctacggggacgtctacacctgcgtggttcagcacagcgggacctctgagcccatccgcggggactggacacctgggctgtcccccatccagacagtgaaggtctctgtgtctgcagccaccctgggcctgggcttcatcatcttctgtgttggcttcttcagatggcgcaagtctcattcctccagctacactcctctccctggatccacctacccagaaggacggcattagagagcagaacccggaaccaaggagattcagactcctgaggattccttactgccccaacatcttcaggacaccccactttcttgtgtcatactgcctctattcaggtccccttcctgaagtaaatattagtagtttgggggggtatttataattaagtctgttttaggtgttttctgagtgagctgaaatgaggcgggggttcaaggcctcctgagagcccctccttgtcatgtgggcaaggtgatcactaagaaataaactggtggacttttgatttct

SEQ ID NO: 10 - protein, 261 aa, Mus musculus
MAALWLLLLVLSLHCMGAGGFVAHVESTCVLDDAGTPQDFTYCVSFNKDLLACWDPIVGKIVPCEFGVLYPLAENFSRILNKEESLLQRLQNGLLDCASHTQPFWNALTHRTRPPSVRVAQTTPFNTREPVMLACYVWGFYPADVTITWMKNGQLVPSHSNKEKTAQPNGDWTYQTVSYLALTPSYGDVYTCVVQHSGTSEPIRGDWTPGLSPIQTVKVSVSAATLGLGFIIFCVGFFRWRKSHSSSYTPLPGSTYPEGRH

SEQ ID NO: 11 - DNA, 1282 bp, Mus musculus
gagtgacttggcgccacataggcagcaaggagcaaggggcctgagcaagaggtaacatatttacctcagtgtgtgaagatcatttgcacaggctggggcaggagagaacctgagacccctgcaggaggtgcccaggggcctggccttcctgcaccttcagagagtggaacatggctgcactctggctgctgctgctggtcctcagtctgcactgtatgggggcaggtggctttgtggctcatgtggaaagcacgtgcgtgctggatgatgctggaaccccacaggacttcacatactgtgtttccttcaacaaagatctgctggcctgctgggatccaatagtgggaaaaatagtcccctgtgaatttggggtgctgtatccattggccgaaaatttttcaaggatcctcaacaaagaagagagtcttctccagcgtttgcaaaacgggcttccggactgtgcctcccacacccagcccttctggaatgcgctgacccacagaacgagaccgccatctgtccgagtagcccaaaccacaccttttaacacaagggagcccgtgatgctggcctgctacgtctggggcttctatccagcggatgtgaccatcacatggatgaagaatgggcagcttgtcccttcccacagcaacaaggagaagacggctcagcccaatggagactggacataccagacagtctcctacctagccctaaccccttcctacggggacgtctacacctgcgtggttcagcacagcgggacctctgagcccatccgcggggactggacacctgggctgtcccccatccagacagtgaaggtctctgtgtctgcagccaccctgggcctgggcttcaccatcttctgtgttggcttcttcagatggcgcaagtctcattcctccagctacactcctctctctggatccacctacccagaaggacggcattagagagcagaacccggaaccaaggagattcagactcctgaggattccttcatgcccccaacatcttcaggacaccccactttcttgtgtcatactgcctctattcaggtccccttcctgaagtaaatattagtagtttgggggggtatttataatgaagtctgtcccaggtgttttctgaggtgagctgaaatgaggtgggggttcaaggcctcctgagagcccctccttgtcacgagggcaccgtgtcttaggtgatcactaagaaataaactggtggacttttaaaaaaaaaaaaaaaaaaaaaaaaaaaaaaaaaaaaaaaaaaa

SEQ ID NO: 12 - protein, 261 aa, Mus musculus
MAALWLLLLVLSLHCMGAGGFVAHVESTCVLDDAGTPQDFTYCVSFNKDLLACWDPIVGKIVPCEFGVLYPLAENFSRILNKEESLLQRLQNGLPDCASHTQPFWNALTHRTRPPSVRVAQTTPFNTREPVMLACYVWGFYPADVTITWMKNGQLVPSHSNKEKTAQPNGDWTYQTVSYLALTPSYGDVYTCVVQHSGTSEPIRGDWTPGLSPIQTVKVSVSAATLGLGFTIFCVGFFRWRKSHSSSYTPLSGSTYPEGRH

SEQ ID NO: 13 - DNA, 1318 bp, Mus musculus
gattcaggttcctcacagacccagggagtgaggatgttgctttttgcccacttgcttcagctgctggtcagcgccacagtcccgacccagagtagcccacactcgctgcggtatttcaccaccgccgtgtcccggcccggcctcggggagccccggttcatcattgtcggctacgtggacgacacgcagttcgtgcgcttcgacagcgacgcggaaaatccgaggatggagcctcgggcgcggtggattgagcaggaggggccggagtattgggagcgggagacttggaaagccagggacatggggaggaacttcagagtaaacctgaggaccctgctcggctactacaatcagagtaacgacgaatctcacacgctgcagtggatgtacggctgcgacgtggggcccgatgggcgcctgctccgcgggtattgtcaggaggcctacgatggccaggattacatctccctgaacgaggacctgcgttcctggaccgcgaatgacatagcctcacagatctctaagcacaagtcagaggcagtcgatgaggcccaccaacagagggcatacctgcaaggtccttgcgtggagtggctccatagatacctacggctgggaaatgagacactgcagcgctcagaccctccaaaggcacatgtgacccatcaccctagatctgaagatgaagtcaccctgaggtgctgggccctgggcttctaccctgctgacatcaccctgacctggcagttaaatggggaggagctgacccaggacatggagcttgtggagaccaggcctgcaggggatggaaccttccagaagtgggcagctgtcgtggtgcctcttgggaaggagcagtattacacatgccatgtgtaccatgaggggctgcctgagcccctcaccctgagatgggagcctcctccatccactgtctccaacatggtaatcatagctgttctggttgtccttggagctgtgatcatccttggagctgtggtggcttttgtgatgaagaggaggagacacataggtgtaaaaggatgctatgctcatgttctaggcagcaagagcttccagacctctgactggcctcagaaggcatgaaaatccctaggggggctggtgagatggctcagtgggtaagagcactgactgctcttctgaaggtccagagttcaaatcccagcaaccacatggtggctcacaaccatccgtaacgagatctgactccctcttctggagtgtctgaagacagctacaatgtacttacatataataaataaataaataaataaataaataaataaataaataa

SEQ ID NO: 14 - protein, 357 aa, Mus musculus
MLLFAHLLQLLVSATVPTQSSPHSLRYFTTAVSRPGLGEPRFIIVGYVDDTQFVRFDSDAENPRMEPRARWIEQEGPEYWERETWKARDMGRNFRVNLRTLLGYYNQSNDESHTLQWMYGCDVGPDGRLLRGYCQEAYDGQDYISLNEDLRSWTANDIASQISKHKSEAVDEAHQQRAYLQGPCVEWLHRYLRLGNETLQRSDPPKAHVTHHPRSEDEVTLRCWALGFYPADITLTWQLNGEELTQDMELVETRPAGDGTFQKWAAVVVPLGKEQYYTCHVYHEGLPEPLTLRWEPPPSTVSNMVIIAVLVVLGAVIILGAVVAFVMKRRRHIGVKGCYAHVLGSKSFQTSDWPQKA

SEQ ID NO: 15 - DNA, 1263 bp, Mus musculus
gctgtaggtctgaaaagggatcctactgcttgcttcctgtttcataaagacacaaatgatgcagtctcaattctcatttctgttggacgcctagttttctaaaagccaatcagcgacgcagaggtttcaaagtatgaagtcaacagaatacggtcctcattcaggttcctcacagacccagggagtgaggatgttgctttttgcccacttgctccagctgctggtcagcgccacagtcccgacccagagtagctcacactcgctgcggtatttccacaccgtcgtatcccggcccggcctcggagagccccggttcatcattgtcggctacgtggacgacacgcagttcgtgcgcttcgacagcgactcggagaatccgaggatggagcctcgggcgcggtggattgagcaggaggggccggagtattgggagcgggagactcggaaagccagggacatggggaggaacttcagagtaaacctgaggaccctgctcggctactacaatcagagtaaggacgaatctcacacgctgcagtggatgtacggctgcgacgtggggcccgatgggcgcctgctccgcgggtattgtcaggaggcctatgatggccaggattacatctccctgaacgaagacctgcgctcctggaccgcgaccaacttagcctcgcatatctctaagtgcaagtcagaggcggtcgatgaggcccaccaacagagggcatacctgcaaggtccttgcgtggagtggctccatacatacctacagctgggaagtgagacactgctgcgctcagaccctccaaaggcacatgtgacccgtcaccccagacctgaaggtgatgtcaccctgaggtgctgggccctgggcttctatcctgctgacatcaccctgacctggcagttgaatggggaggagctgacccaggacatggagcttgtggagaccaggcctgcaggggatggaaccttccagaagtgggcatctgtggtggtgcctcttgggaaggagcagaattacacatgccatgtgtaccatgaggggctgcctgagcccctcaccctgagatgggagcctcctccatccactgtctccaacatggtaatcattgctgttctggttgtccttggagctgtgatagtcattgttgctgtggtggcttttgtaatgaagaggaggaaaacacaggtgtaaaaggatgctatgctcatgttctaggcagcaagagcttccagacctctgactggcctcagaaggcatga

SEQ ID NO: 16 - protein, 334 aa, Mus musculus
MLLFAHLLQLLVSATVPTQSSSHSLRYFHTVVSRPGLGEPRFIIVGYVDDTQFVRFDSDSENPRMEPRARWIEQEGPEYWERETRKARDMGRNFRVNLRTLLGYYNQSKDESHTLQWMYGCDVGPDGRLLRGYCQEAYDGQDYISLNEDLRSWTATNLASHISKCKSEAVDEAHQQRAYLQGPCVEWLHTYLQLGSETLLRSDPPKAHVTRHPRPEGDVTLRCWALGFYPADITLTWQLNGEELTQDMELVETRPAGDGTFQKWASVVVPLGKEQNYTCHVYHEGLPEPLTLRWEPPPSTVSNMVIIAVLVVLGAVIVIVAVVAFVMKRRKTQV

SEQ ID NO: 17 - DNA, 1263 bp, Mus musculus
gctgtaggtctgaaaagggatcctactgcttgcttcctgtttcataaagacacaaatgatgcagtctcaattctcatttctgttggacgcctagttttctaaaagccaatcagcgacgcagaggtttcaaagtatgaagtcaacagaatacggtcctcattcaggttcctcacagacccagggagtgaggatgttgctttttgcccacttgctccagctgctggtcagcgccacagtcccgacccagagtagctcacactcgctgcggtatttccacaccgtcgtatcccggcccggcctcggagagccccggttcatcattgtcggctacgtggacgacacgcagttcgtgcgcttcgacagcgactcggagaatccgaggatggagcctcgggcgcggtggattgagcaggaggggccggagtattgggagcgggagactcggaaagccagggacatggggaggaacttcagagtaaacctgaggaccctgctcggctactacaatcagagtaaggacgaatctcacacgctgcagtggatgtacggctgcgacgtggggcccgatgggcgcctgctccgcgggtattgtcaggaggcctatgatggccaggattacatctccctgaacgaagacctgcgctcctggaccgcgaccaacttagcctcgcatatctctaagtgcaagtcagaggcggtcgatgaggcccaccaacagagggcatacctgcaaggtccttgcgtggagtggctccatacatacctacagctgggaagtgagacactgctgcgctcagaccctccaaaggcacatgtgacccgtcaccccagacctgaaggtgatgtcaccctgaggtgctgggccctgggcttctatcctgctgacatcaccctgacctggcagttgaatggggaggagctgacccaggacatggagtttgtggagaccaggcctgcaggggatggaaccttccagaagtgggcatctgtggtggtgcctcttgggaaggagcagaattacacatgccatgtgtaccatgaggggctgcctgagcccctcaccctgagatgggagcctcctccatccactgtctccaacatggtaatcattgctgttctggttgtccttggagctgtgatagtcattgttgctgtggtggcttttgtaatgaagaggaggaaaacacaggtgtaaaaggatgctatgctcatgttctaggcagcaagagcttccagacctctgactggcctcagaaggcatga

SEQ ID NO: 18 - protein, 334 aa, Mus musculus
MLLFAHLLQLLVSATVPTQSSSHSLRYFHTVVSRPGLGEPRFIIVGYVDDTQFVRFDSDSENPRMEPRARWIEQEGPEYWERETRKARDMGRNFRVNLRTLLGYYNQSKDESHTLQWMYGCDVGPDGRLLRGYCQEAYDGQDYISLNEDLRSWTATNLASHISKCKSEAVDEAHQQRAYLQGPCVEWLHTYLQLGSETLLRSDPPKAHVTRHPRPEGDVTLRCWALGFYPADITLTWQLNGEELTQDMEFVETRPAGDGTFQKWASVVVPLGKEQNYTCHVYHEGLPEPLTLRWEPPPSTVSNMVIIAVLVVLGAVIVIVAVVAFVMKRRKTQV

SEQ ID NO: 19 - DNA, 1263 bp, Mus musculus; identical to SEQ ID NO: 17

SEQ ID NO: 20 - protein, 334 aa, Mus musculus; identical to SEQ ID NO: 18

SEQ ID NO: 21 - DNA, 1300 bp, Mus musculus
gctgtaggtcatgaaaagggatcctactgctttcttcctctttcataaagacacaaatgatgcagtctcaattctcatttctgttggacgcctagttttctaaaagccaatcagcgacgcagaggtttcaaagtatgaagtcaacaggaatacggtcctcattcaggttcctcacagacccagggagtgaggatgttgctttttgcccacttgctccagctgctggtcagcgccacagtcccgacccagagtagctcacactcgctgcggtatttccacaccgtcgtatcccggcccggcctcggagagccccggttcatcattgtcggctacgtggacgacacgcagttcgtgcgcttcgacagcgactcggagaatccgaggatggagcctcgggcgcggtggattgagcaggaggggccggagtattgggagcgggagactcggaaagccagggacatggggaggaacttcagagtaaacctgaggaccctgctcggctactacaatcagagtaaggacgaatctcacacgctgcagtggatgtacggctgcgacgtggggcccgatgggcgcctgctccgcgggtattgtcaggaggcctatgatggccaggattacatctccctgaacgaagacctgcgctcctggaccgcgaccaacttagcctcgcatatctctaagtgcaagtcagaggcggtcgatgaggcccaccaacagagggcatacctgcaaggtccttgcgtggagtggctccatacatacctacagctgggaagtgagacactgctgcgctcagaccctccaaaggcacatgtgacccgtcaccccagacctgaaggtgatgtcaccctgaggtgctgggccctgggcttctatcctgctgacatcaccctgacctggcagttggaatggggaggagctgaccaggacatggagtttgtggagaccaggcctgcaggggatggaaccttccagaagtgggcatctgtggtggtgcctcttgggaaggagcagaattacacatgccatgtgtaccatgaggggctgcctgagcaccctcaaaccctagatggggtaagaagggtgtgggtgcagatctgggagcctcctccatccactggtctccaacatggtaatcattgctgttctggttgtccttggagctgtgatagtcattgttgctgtggtggcttttgtaatgaagaggaggaaaacacaggtaggtgtaaaaggatgctatgctcatgttctaggcagcaagagcttccagacctctgactggcctcagaaggcatga

SEQ ID NO: 22 - DNA, 796 bp, Mus musculus
gacatgatgcaaacaaaaattccaacagcctccagatatgattagggttcctcccctgacaagacctatctttgcagtctttgcctccttggcctctaggtgggcagcagcagtcagctctgccagggaagatggctgagtacgagttcatgtatatccactccctggctgagcactatcttcagtatgtgctacaggtacccgcctttgagtcggctccaagccaagcatgcagagtgctacaaagagttgctttctccgttcagaaggaagttgaaaagaatctgaagtcatacttggatgactttcacgtggaatccatagataccgccagaataatattcaaccaagtgatggaaaaagagtttgaagatggcatcattaactggggaaggattgtgactatatttgcctttgggggtgttctcctcaaaaaacttccgcaagagcagattgccctggatgtaggtgcttacaaacaagtttccagttttgtggcagaattcataatcaataacacaggagaatggatacggcggaatggaggttgggaagatggcttcataaagaagtttgaacccaaatctggctggctgacttttctgcagatgacaggacagttctgggaaatgctctttctcctcaagtaacactacggaccagagagaaccttggctcccatgagaatggttatcagcacttggcatttccatttacaaacatgaaacttgtctttccagagttgcagatattttgtttcaactttaatgaataaattactagtatttgtttctgt

SEQ ID NO: 23 - protein, 172 aa, Mus musculus
MAEYEFMYIHSLAEHYLQYVLQVPAFESAPSQACRVLQRVAFSVQKEVEKNLKSYLDDFHVESIDTARIIFNQVMEKEFEDGIINWGRIVTIFAFGGVLLKKLPQEQIALDVGAYKQVSSFVAEFIINNTGEWIRRNGGWEDGFIKKFEPKSGWLTFLQMTGQFWEMLFLLK

SEQ ID NO: 24 - DNA, 796 bp, Mus musculus
gacatgatgcaaacagaaattccaacagcctccagatatgattagggttcctcccctgacaagacctatctttgcagtctttgcctccttggcctctaggtgggcagcagcagtcagctctgccagggaagatgtctgagtacgagttcatgtatatccactccctggctgagcactaccttcagtatgtgctacaggtacccgcctttgagtcggctccaagccaagcatgcagagtgctacaaagagttgctttctccgttcagaaggaagttgaaaagaatctgaagtcatacttggatgactttcacgtggaatccatagataccgccagaataatattcaaccaagtgatggaaaaagagtttgaagatggcatcattaactggggaaggattgtgactatatttgcctttgggggtgttctcctcaaaaaacttccacaagagcagattgccctggatgtaggtgcttacaaacaagtttccagttttgtggcagaattcataatgaataacacaggagaatggatacggcggaatggaggttgggaagatggcttcataaagaagtttgaacccaaatctggctggctgacttttctgcagatgacaggacagatctgggaaatgctctttctcctcaagtaacactacggaccagagagaaccttggctcccacgagaatggttatcagcacttggcatttccatttacaaacatgaaacttgtctttccagagttgcagatattttgtttcaactttaatgaataaattactagtatttgtttctgt

SEQ ID NO: 25 - protein, 172 aa, Mus musculus
MSEYEFMYIHSLAEHYLQYVLQVPAFESAPSQACRVLQRVAFSVQKEVEKNLKSYLDDFHVESIDTARIIFNQVMEKEFEDGIINWGRIVTIFAFGGVLLKKLPQEQIALDVGAYKQVSSFVAEFIMNNTGEWIRRNGGWEDGFIKKFEPKSGWLTFLQMTGQIWEMLFLLK

SEQ ID NO: 26 - DNA, 796 bp, Mus musculus
gacatgatgcaaacagaaattccaacagcctccagatatgattagggttcctcccctgacaagacctatctttgcagtctttgcctccttggcctctaggtggacagctgcagtcagctctgccagggaagatggctgagtctgagctcatgcatatccactccctggctgagcactaccttcagtatgtgctacaggtacccgcctttgagtcggctccaagccaagcatgcagagtgctacaaagagttgctttctccgttcagaaggaagttgaaaagaatctgaagtcatacttggatgactttcacgtggaatccatagataccgccagaataatattcaaccaagtgatggaaaaagagtttgaagatggcatcattaactggggaaggattgtgactatatttgcctttgggggtgttctcctcaaaaaacttccacaagagcagattgccctggatgtatgtgcttacaaacaagtttccagttttgtggcagaattcataatgaataacacaggagaatggatacggcagaatggaggttgggaagatggcttcataaagaagtttgaacccaaatctggctggctgacttttctgcagatgacaggacagatctgggaaatgctctttctcctcaagtaacactacggaccagagagaaccttggctcccacgagaatggttatcagcacttggcatttccttttacaaacatgaaacttgtctttccagagttgcagatattttgtttcaactttaatgaataaattactagtatttgtttctgt

SEQ ID NO: 27 - protein, 172 aa, Mus musculus
MAESELMHIHSLAEHYLQYVLQVPAFESAPSQACRVLQRVAFSVQKEVEKNLKSYLDDFHVESIDTARIIFNQVMEKEFEDGIINWGRIVTIFAFGGVLLKKLPQEQIALDVCAYKQVSSFVAEFIMNNTGEWIRQNGGWEDGFIKKFEPKSGWLTFLQMTGQIWEMLFLLK

SEQ ID NO: 28 - DNA, 2863 bp, Mus musculus
tggcctcagcggctgtgttctttagacacaaccacacagccagctggcaagaagtaggcaaagacatctccagtcagattctcggagtggaggcttcctgggactgagagggggacatgctgggcccacactcactgctgcttgccctagctggactgttcttcctgggatctgctgtgtcccaggaatgcaccaagtacaaagtcagcagttgccgggactgtatccagtcggggcctggctgttcctggtgccagaagctgaacttcactggaccaggagaacctgactccttgcgctgtgacacacgggcacagctgctgctgaagggttgtccagccgatgatatcatggaccccaggagcatcgctaatcctgagttcgaccaacgggggcaacggaaacagctatctccacaaaaagtgacactttacttgcgaccaggacaggctgccgcattcaatgtgactttccggcgggccaagggataccccattgatctgtactacctcatggatctctcctactccatgcttgatgacctcaacaacgtcaagaagctgggcggggacttgctgcaggccctcaacgagatcaccgagtctggccgcatcggctttgggtcgtttgtggacaagacggtgctgccttttgttaacacccatcctgagaagctgaggaacccatgtcccaacaaggagaaggcctgccagcccccatttgcctttcggcacgtgctcaagttaaccgacaactccaaccagtttcagacagaggtcggcaagcaactgatttccggaaacctggacgcccctgagggtgggctggatgccataatgcaagttgctgcatgtccggaggaaattggctggcgcaatgtcacgaggctgctggtgtttgccacagacgatggcttccactttgctggtgatggcaaactgggtgccatcctgacccccaatgatggccgctgccacctggaggataacatgtacaagaggagcaatgagttcgactacccatccgtgggtcagctggcacacaaactttccgagagcaacatccagcccatctttgcggtgacaaagaagatggtgaaaacgtatgagaaactcacggagatcatccccaagtcagcagtgggggaactgtctgacgactccagcaacgtggtgcagctcatcaagaatgcctactataaactctcctctagagtcttcctggaccacagcaccctcccggacaccctgaaagtcacctatgactccttctgcagtaatggagcatcgagtataggcaaatcccgtggggactgtgatggcgtacagatcaacaacccggtcaccttccaggtaaaggtcatggcttccgagtgtatccaggagcagtcctttgtcatccgggcactgggtttcacggatacagtgaccgtgcaggtccgtccccagtgtgagtgtcagtgccgggaccagagtcgggagcagagtctctgtggaggcaagggagtcatggagtgtggtatctgcaggtgtgagtctggctacattgggaaaaactgtgagtgccagactcagggtcggagcagccaggagctggagagaaactgtcggaaggacaatagttccatcgtgtgctcagggcttggggactgcatctgtgggcagtgtgtatgccataccagtgacgtccccaacaaagagatctttgggcaatactgcgagtgtgacaatgtcaactgtgagagatataacagccaagtctgcggtggctcagatcggggttcctgcaactgtggcaaatgtagttgcaagcccggttacgagggctcggcctgccagtgtcagaggtccaccacgggctgtctgaatgcacggctggtagagtgcagtggccgtggccactgccaatgcaacaggtgcatatgtgacgaaggctaccagccaccgatgtgtgaggattgtcccagctgtggctcgcactgcagggacaaccacacctcttgtgccgagtgcctgaagtttgataagggcccttttgagaagaactgtagtgttcagtgtgctggtatgacgctgcagactatccctttgaagaaaaagccctgcaaggagagggactcggaaggctgttggataacttacactttgcagcagaaggacggaaggaacatttacaacatccatgtggaggacagtctagagtgtgtgaagggccccaatgtggctgccatcgtagggggcaccgtggtaggtgtcgtactgattggtgtcctcctcctggtcatctggaaggccctgacccacctgactgacctcagggagtacaggcgctttgagaaggagaaactcaagtcccaatggaacaatgacaaccccctcttcaagagtgctacgacaacggtcatgaaccccaagtttgctgaaagctagagcatgagttatcataatcaagcagatgtgaccccctcagaccacgcctcctcccctctgcaaacacaacgtggcttacagctcaccccagtgctgccaaggatccaaaagcctgctcggtttctttccgccattatatcaagtctgccagggtttccagggacttgtcttccgacctgcacaatcttgccgcagagccctaagaattgtcccgagtcccaagaggttccacccacattttcttgcataaaggaagacagcagtctcagtaaaggtggccccaacttatttatatttaaacttgtcagagtataaaactcctattatattgttaacatcccatctgttgtattatatgtgagtataaaaactatatccaacgtattatttcataatcatgtatgaaaaataataaagcttccatccatgctgcc

SEQ ID NO: 29 - protein, 770 aa, Mus musculus
MLGPHSLLLALAGLFFLGSAVSQECTKYKVSSCRDCIQSGPGCSWCQKLNFTGPGEPDSLRCDTRAQLLLKGCPADDIMDPRSIANPEFDQRGQRKQLSPQKVTLYLRPGQAAAFNVTFRRAKGYPIDLYYLMDLSYSMLDDLNNVKKLGGDLLQALNEITESGRIGFGSFVDKTVLPFVNTHPEKLRNPCPNKEKACQPPFAFRHVLKLTDNSNQFQTEVGKQLISGNLDAPEGGLDAIMQVAACPEEIGWRNVTRLLVFATDDGFHFAGDGKLGAILTPNDGRCHLEDNMYKRSNEFDYPSVGQLAHKLSESNIQPIFAVTKKMVKTYEKLTEIIPKSAVGELSDDSSNVVQLIKNAYYKLSSRVFLDHSTLPDTLKVTYDSFCSNGASSIGKSRGDCDGVQINNPVTFQVKVMASECIQEQSFVIRALGFTDTVTVQVRPQCECQCRDQSREQSLCGGKGVMECGICRCESGYIGKNCECQTQGRSSQELERNCRKDNSSIVCSGLGDCICGQCVCHTSDVPNKEIFGQYCECDNVNCERYNSQVCGGSDRGSCNCGKCSCKPGYEGSACQCQRSTTGCLNARLVECSGRGHCQCNRCICDEGYQPPMCEDCPSCGSHCRDNHTSCAECLKFDKGPFEKNCSVQCAGMTLQTIPLKKKPCKERDSEGCWITYTLQQKDGRNIYNIHVEDSLECVKGPNVAAIVGGTVVGVVLIGVLLLVIWKALTHLTDLREYRRFEKEKLKSQWNNDNPLFKSATTTVMNPKFAES

SEQ ID NO: 30 - DNA, 1285 bp, Mus musculus
ggagttactgggtacaagtttaggatctcaggaggggctgagtgacttggcgccacataagcagcaaggagcaaggggcctgagcaagaggtaccatatttacctcagtgtgtgaagatcatttgtccaggctggggcaggagagaacctgaggcccctgcaggaggtgcccaggggcctggccttcctgcaccttcagagagtgaaacatggctgcactctggctgctgctgctggtcctcagtctggactgtatgggggcaggtggctttgtggctcatgtggaaagcacgtgcgtgctggatgatgctgggaccccacaggacttcacatactgtgtttccttcaacaaagatctgctggcctgctgggatccagatgtgggaaaaatagtcccctgtgaatttggggtgctgtatccatgggccgaaaatttttcaaggatcctcaacaaagaagagagccttctccagcgtttgcaaaacgggcttctggactgtgcctcccacacccagcccttctggaatgcgctgacccacagaacgagagcgccatctgtccgagtagcccaaaccacaccttttaacacaagggagccggtgatgctggcctgctacgtctggggcttctatccagcggatgtgaccatcacatggatgaagaatgggcagcttgtcccttcccacagcaacaaggagaagacggctcagcccaatggagactggacataccagacagtctcctacctagccctaaccccttcctacggggacgtctacacctgcgtggttcagcacagcgggacctctgagcccatccgaggggactggacacctgggctgtcccccatccagacagtgaaggtctctgtgtctgcagccaccctgggcctgggcttcatcatcttctgtgttggcttcttcagatggcgcaagtctcattcctccagctacactcctctctctggatccacctacccggaaggacagcattagagagcagaacccggaaccaaggagattcagactcctgaggattccttaatgccccaacatcttcaggacaccccactttcttgtgtcatactgcctctattcaggtccccttcctgaaataaatattagtagtttgggggggtatttataatgaagtctgtcccaggtgggggagctgaggtgagctgaaatgaggtgggggttcaaggcctcctgagagcccctccttgtcacgagggcaccgtgtcttaggtgatcactaagaaataaactggtggacttttgatttc

SEQ ID NO: 31 - protein, 261 aa, Mus musculus
MAALWLLLLVLSLDCMGAGGFVAHVESTCVLDDAGTPQDFTYCVSFNKDLLACWDPDVGKIVPCEFGVLYPWAENFSRILNKEESLLQRLQNGLLDCASHTQPFWNALTHRTRAPSVRVAQTTPFNTREPVMLACYVWGFYPADVTITWMKNGQLVPSHSNKEKTAQPNGDWTYQTVSYLALTPSYGDVYTCVVQHSGTSEPIRGDWTPGLSPIQTVKVSVSAATLGLGFIIFCVGFFRWRKSHSSSYTPLSGSTYPEGQH

SEQ ID NO: 32 - DNA, 1348 bp, Mus musculus
gagtgtgtgaagatcatttgcccaggctggggcaggagagaacctgaggcccttgcaggaggtgcccaggggcctggccttcctgcaccttcagagagtggaacatggctgcactctggctgctgctgctggtcctcagtctgcactgtatgggggcaggtggctttgtggctcatgtggaaagcacgtgcgtgctgaatgatgctgggaccccacaggacttcacatactgtgtttccttcaacaaagatctgctggcctgctgggatccagatgtgggaaaaatagtcccctgtgaatttggggtgctgtctagattggctgaaataatttcaaatatcctcaacgaacaggagagccttattcatcgcttgcaaaacgggcttcaggactgtgccacccacacccaacctttctgggatgtgctgacccacagaacgagagcgccatctgtccgagtagcccaaaccacaccttttaacacaagggagcccgtgatgctggcctgctacgtctggggcttctatccagcggatgtgaccatcacatggatgaagaatgggcagcttgtcccttcccacagcaacaaggagaagacggctcagcccaatggagactggacataccagacagtctcctacctagccctaaccccttcctacggggacgtctacacctgcgtggttcagcacagcgggacctctgagcccatccgcggggactggacacctgggctgtcccccatccagacagtgaaggtctctgtgtctgcagccaccctgggcctgggcttcatcatcttctgtgttggcttcttcagatggcgcaagtctcattcctccagctacactcctctccccggatccacctacccagaaggacggcactagaaagcagaacccagaaccaaggagattcagactcctgaggattccttcatgcccccaatatcttcaggacaccccattttcttgtgtcatactgcctctattcagtttcccttcccaatgtaattttttttttgttttttgtttttttgagacagggtttctctgtatagccctggctgtcctggaactcactttgtagaccaggctggcctcaaactcagaaatccacctgcctctgcctccagagtgctgggattaaaggcgtacgccaccatgcccggcttccgatgtgtaacagcttgggaggtattcataatgaagtctgtctcaggtgttttctgaagtgagctgatctaaggagggggttcaaggtcttctgagagcccctccttgtcacaagggcaccgtgtcataggtggtcactaagaaataaactggtggaattcctggcttccccc

SEQ ID NO: 33 - protein, 261 aa, Mus musculus
MAALWLLLLVLSLHCMGAGGFVAHVESTCVLNDAGTPQDFTYCVSFNKDLLACWDPDVGKIVPCEFGVLSRLAEIISNILNEQESLIHRLQNGLQDCATHTQPFWDVLTHRTRAPSVRVAQTTPFNTREPVMLACYVWGFYPADVTITWMKNGQLVPSHSNKEKTAQPNGDWTYQTVSYLALTPSYGDVYTCVVQHSGTSEPIRGDWTPGLSPIQTVKVSVSAATLGLGFIIFCVGFFRWRKSHSSSYTPLPGSTYPEGRH

SEQ ID NO: 34 - DNA, 5217 bp, Mus musculus
aggaggcgggacgccctctagccttttttcctgcccggtactttcggttttcagcgccgagtctgtcaaagctccctggagacctccgggaccgcgcccctcagacccacttgggacactgctgagcggcgcagagagatttgcccagactcgagctcctgcgtgcagtgagtgagtgagagccagtcgtttcagctctgctccataccctgagccggcgccacgccgccgcgcatgcaactggcatataacttgctgtgtgtggtgattgcttgtgttgaatcccgaacctgcacccggagacagcccagtaagtctacgtgggaacggaagcatttggaatctcaaggatgtcacagtggttcgagcttcagcagctggactccaagttcctggagcaggtccaccagctgtacgatgacagtttccccatggaaatcagacagtacctggcccagtggctggaaaagcaagactgggagcacgctgcctatgatgtctcgtttgcgaccatccgcttccatgacctcctctcacagctggacgaccagtacagccgcttttctctggagaataatttcttgttgcagcacaacatacggaaaagcaagcgtaatctccaggataacttccaagaagatcccgtacagatgtccatgatcatctacaactgtctgaaggaagaaaggaagattttggaaaatgcccaaagatttaatcaggcccaggagggaaatattcagaacactgtgatgttagataaacagaaggagctggacagtaaagtcagaaatgtgaaggatcaagtcatgtgcatagagcaggaaatcaagaccctagaagaattacaagatgaatatgactttaaatgcaaaacctctcagaacagagaaggtgaagccaatggtgtggcgaagagcgaccaaaaacaggaacagctgctgctccacaagatgtttttaatgcttgacaataagagaaaggagataattcacaaaatcagagagttgctgaattccatcgagctcactcagaacactctgattaatgacgagctcgtggagtggaagcgaaggcagcagagcgcctgcatcgggggaccgcccaacgcctgcctggatcagctgcaaagctggttcaccattgttgcagagaccctgcagcagatccgtcagcagcttaaaaagctggaggagttggaacagaaattcacctatgagcccgaccctattacaaaaaacaagcaggtgttgtcagatcgaaccttcctcctcttccagcagctcattcagagctccttcgtggtagaacgacagccgtgcatgcccactcacccgcagaggcccctggtcttgaagactggggtacagttcactgtcaagctgagactgttggtgaaattgcaagagctgaactataacttgaaagtgaaagtctcatttgacaaagatgtgaacgagaaaaacacagttaaaggatttcggaagttcaacatcttgggtacgcacacaaaagtgatgaacatggaagaatccaccaacggaagtctggcagctgagttccgacacctgcaactgaaggaacagaaaaacgctgggaacagaactaatgaggggcctctcattgtcaccgaagaacttcactctcttagctttgaaacccagttgtgccagccaggcttggtgattgacctggagaccacctctcttcctgtcgtggtgatctccaacgtcagccagctccccagtggctgggcgtctatcctgtggtacaacatgctggtgacagagcccaggaatctctccttcttcctgaaccccccgtgcgcgtggtggtcccagctctcagaggtgttgagttggcagttttcatcagtcaccaagagaggtctgaacgcagaccagctgagcatgctgggagagaagctgctgggccctaatgctggccctgatggtcttattccatggacaaggttttgtaaggaaaatattaatgataaaaatttctccttctggccttggattgacaccatcctagagctcattaagaagcacctgctgtgcctctggaatgatgggtgcattatgggcttcatcagcaaggagcgagaacgcgctctgctcaaggaccagcagccagggacgttcctgcttagattcagtgagagctcccgggaaggggccatcacattcacatgggtggaacggtcccagaacggaggtgaacctgacttccatgccgtggagccctacacgaaaaaagaactttcagctgttactttcccagatattattcgcaactacaaagtcatggctgccgagaacataccagagaatcccctgaagtatctgtaccccaatattgacaaagaccacgcctttgggaagtattattccagaccaaaggaagcaccagaaccgatggagcttgacgaccctaagcgaactggatacatcaagactgagttgatttctgtgtctgaagtccacccttctagacttcagaccacagacaacctgcttcccatgtctccagaggagtttgatgagatgtcccggatagtgggccccgaatttgacagtatgatgagcacagtataaacacgaatttctctctggcgacatttttttcccatctgtgattccttcctgctactgttccttcatatgcagtatttctagggaaatgcaagaaagaaagagcatcacatttgctgagcactgctggtagaaagtggatatttctctaattagaaacctgttactctgaaggacttcatgcatcttactgaaggtgaaatggaaagtcacttaacacaaaatggattttgtaaacaaagaccaagagatccacccaagcaccaggactagagtgcgagtatttggggcaaggtgaggagaacggtcactttagtaatggtctgtaatcagtgcccaagtgctgcacatcactggaaagagacatacttatgggggaggggccttcttgatggaggaatgtttctgtcccgggagacattggcacttcccctctcctggatggccggaagtcttccactgttttacatatggcacagttcaaagtcaactttagatccaatgctctatcaaactatagtgggcatccttcatgtgagtgggaagaaaacaaccgtgctccttactgcagcttctgccaaggcatggttgctctcctcagggactagctttgttggtggcaatggctacacaaaactaaacaccaacagaagtaagaccattttcatgagtactccatcaagttaaagggtttttgttgtctttttggtcatggattgaataaaattgtctttgcacatccattaagggggccagctttcttaaagcaatttttctttttttttaactaaaattagatataggtgaactcatgttttttagtgggctgaacttatcggttttagctggttgtcttaattagccataaacttggagaaagcagtgacttcttgaatccttagccaaatatgagtatcagataattttattatttttttttcgagacagggtttctctgtgtagccctggctattctggaactcactctgtagatcaggctggcctggaactcagaaatccgcctgtctctgcctcccgagtgctgggattaaaggtgtgcaccaccaatgcctggtgagataactttaaagaactccctataaatgcatgagaaccactgttactgatgaatgtggttttttgacaactacattcacaaatggcctgtcttgtgttttgtcaccgttttgagggatgatgttttgtggcacgtgtgtgatcacagcctgatggttctggtcgtgggttggttcttctgggccagctttcacagactgctgcgcagctgcacctacagtgctgccccataatactgtttcactttggtgaagatcagcccaccttacaccccgagtgcaggtgtgaaccacggtaagtgtgcacagtccttagggaaaacagggacgcagaggcctgcctcctctcttttccatgccaaaatgaaatgaccaagaaacaaaacatttaaaaagttgtttctaaatgctgagacctaaccattgcttatatactgttgtctgttgaaacagtttgttacaatttcattctgttgaactaggtgagactttaagaaatgttgaaattatgttaatttcctattattatttaatataaagatatttaaaatgtctagtgttatgagttggtttaatatatatctcatgtatgtatcagtcctattttaagcgctttttaaaaaagacttgtttaggtatatattttatgtatatgggtgttttatctgaatgtatggacaccagaagagagcactgggtcccatgagactacagtcaaatgggatcacagtcagatgcaattttgagctgccatgtgggtgctgggatttgaactcagggcctctggaagaacagccagtgcttttaactgctgaaccatctcttgggacgcctatgggtcctattttaaaggaagaccgatatttcccaagtgtcacccttgcttactctactgcagacacaggaactatggcagtttaaggtaggtcagcgggcaggcccagcggtgaggcctctctgaggtagaggcctggggatcaggcgtttagtcattcaaggccggcagggctccatgggatcacatccaagtcagggcaagacatccacttacactttcaggtagctgcctgttagacgtcaccaagcatgtcctagcttaagtatgacgaggatggcattttctaagtacagagcatatatgtagcacttgggataagctgatctagggacttcctgaggcggcagagcttaaagtggactccattgactgcttggagactactgtggaatgaaggcatgtctcagtaaacagaacaaacctgtggcatctgtctgcctcagggaggcatcagacaaagctggaaaggatttagaaaattcttttacctactctgggagctgggaggctgtccttcatcccgagggcattatttatctgaaggcatcttttgtttaagattcattcatttgttcctcaaatatgttcagtgcctgccttgcaccaggcactattctagatgcgttgaaggcactaataaaagaaatgcctcacaaaaaaaaaaaaaaaaaaaaaaaaaaaaaaaaaaaaaaaaaaaaaaaaaaaa

SEQ ID NO: 35 - protein, 749 aa, Mus musculus
MSQWFELQQLDSKFLEQVHQLYDDSFPMEIRQYLAQWLEKQDWEHAAYDVSFATIRFHDLLSQLDDQYSRFSLENNFLLQHNIRKSKRNLQDNFQEDPVQMSMIIYNCLKEERKILENAQRFNQAQEGNIQNTVMLDKQKELDSKVRNVKDQVMCIEQEIKTLEELQDEYDFKCKTSQNREGEANGVAKSDQKQEQLLLHKMFLMLDNKRKEIIHKIRELLNSIELTQNTLINDELVEWKRRQQSACIGGPPNACLDQLQSWFTIVAETLQQIRQQLKKLEELEQKFTYEPDPITKNKQVLSDRTFLLFQQLIQSSFVVERQPCMPTHPQRPLVLKTGVQFTVKLRLLVKLQELNYNLKVKVSFDKDVNEKNTVKGFRKFNILGTHTKVMNMEESTNGSLAAEFRHLQLKEQKNAGNRTNEGPLIVTEELHSLSFETQLCQPGLVIDLETTSLPVVVISNVSQLPSGWASILWYNMLVTEPRNLSFFLNPPCAWWSQLSEVLSWQFSSVTKRGLNADQLSMLGEKLLGPNAGPDGLIPWTRFCKENINDKNFSFWPWIDTILELIKKHLLCLWNDGCIMGFISKERERALLKDQQPGTFLLRFSESSREGAITFTWVERSQNGGEPDFHAVEPYTKKELSAVTFPDIIRNYKVMAAENIPENPLKYLYPNIDKDHAFGKYYSRPKEAPEPMELDDPKRTGYIKTELISVSEVHPSRLQTTDNLLPMSPEEFDEMSRIVGPEFDSMMSTV

SEQ ID NO: 36 - DNA, 491 bp, Mus musculus
cagccaggttcaaagtggcctgcagactgtcctgaactcatctctcaaagctgctccagagcccaggaagatttcaggatgaagagcttcctcctcttcctcactatcattcttctggttgtgattcagatacaaacaggatccttgggacaagccactacggccgcttctggtactaacaaaaacagcacctccaccaaaaaaacccccttaaagagtggggcctcatccatcatcgatgcgggtgcctgcagtttcctcttctttgccaataccttaatgtgcctcttctacctcagctgaggtgaaggcgcagcttctgaagccccgtgctccacccactgctgccaccatcagtagcgagagacatcccaacccacgggaagggttgatagcagatatccaaggaggctaaagttgacagccaatggccggaatgggagcggggccgtgacacccagctagggcacaataaagttatacttatgctt

SEQ ID NO: 37 - protein, 74 aa, Mus musculus
MKSFLLFLTIILLVVIQIQTGSLGQATTAASGTNKNSTSTKKTPLKSGASSIIDAGACSFLFFANTLMCLFYLS

SEQ ID NO: 38 - DNA, 1148 bp, Mus musculus
  misc_feature (76)..(76): n is a, c, g, or t
gaaaagttacagtctgaagcttgccttcccatctgactcctgtgtctcctctcctgcagcatagtgtggaaccccnaaattccctgtgtggcagctgtgttcttgttgcagncaaagctgaacccaccagaggctatggacaaagactccagaccatggttttaaggntncagaaaatctgagagtatatacaacaacgggacacagcgcgtgcggcttctggaaagatacttctacaacctggaggagaacctgagcttcaacaacgaagtgggcgaattccgcgaggtgaccgagctggggcggccagacgccgagaactggaacagacagccggaattccaagagcaaaaacaggccgaagtagagccaatgagcagacacaactatgagatctcggataaattccttgtgcggaggagagttgagctacggtgactgtgtaccccacaaagacacagcccctggaacaccacaacctcctggtctgctctgtgagtgacttctaccctggcaacattgaagtcagatggttccggaatggcaaggaggagaaaacaggaattgtgtccacgggcctggtccgaaatggagactggaccttccagacactggtgatgctggagacggttcctcagagtggagaggtttacacctgccaggtggagcatcccagcctgaccgaccctgtcacggtcgagtggaaagcacaatccacatctgcacagaacaagatgttgagtggagttgggggcttcgtgctgggcctgctcttcctgggagcggggctgttcatctacttcaggaaccagaaaggacagtctggacttcagccaacaggactcctgagctgagatgaagtaacaaggctgaaggaaggaattccccccgtgtctccatgccatgaaaacatgtcctgcttggcccacatccctccagagacactgctcttccaggacctggctcctcctgattctccaccctggagatctgtgctcctgatggctgtttatccctgacccaggccttgcagctcccaggacagaggccccactcttcacatctcctgtccccttttgtcccttgccttttgtctggcacttctgagccagtctgctgtcatatgcttttttacatttttctcaaat

SEQ ID NO: 39 - DNA, 1167 bp, Mus musculus
gcgagttacagtctgaagcttgccttcccctctgactcctgtgtctcctctcctgcagcatggtgtggctccccagagttccctgtgtggcagctgtgatcctgttgctgacagtgctgagccctccagtggctttggtcagagactccagaccatggtttttggaatactgtaaatctgagtgtcatttctacaacgggacgcagcgcgtgcggtttctgaaaagatacttctacaacctggaggagaacctgcgcttcgacagcgacgtgggcgagttccgcgcggtgaccgagctggggcggccagacgccgagaactggaacagccagccggagatcctggagcaaaagcgggccgcggtggacacgtactgcagacacaactatgagatcttcgataacttccttgtgcggcggagagttgagcctacggtgactgtgtaccccacaaagacgcagcccctggaacaccacaacctcctggtctgctctgtgagtgacttctaccctggcaacattgaagtcagatggttccggaatggcaaggaggagaaaacaggaattgtgtccacgggcctggtccgaaatggagactggaccttccagacactggtgatgctggagacggttcctcagagtggagaggtttacacctgccaggtggagcatcccagcctgaccgaccctgtcacggtcgagtggaaagcacagtccacatctgcacagaacaagatgttgagtggagttgggggcttcgtgctgggcctcctcttcctcggagcggggctgttcatctacttcaggaaccagaaaggacagtctggacttcagccaacaggactcctgagctgagatgaagtaacaaggctgaaggaaggagttcccccccgtgtctccatgccatgaaaacatgtcctgcttggcccacatccctccagagacactgctcttccaggacctggctcctcctgattctccaccctggagatctgtgctcctgatggctgcttatccctgacccaggccttgcagctcccagaacagaggccccactcttcacatctcctgtccccttttgtcccttgccttttgtctggcacttctgagccagtctgctgtcatatgcttttttacatttttctcaaataaacaaataatgaaagtc

SEQ ID NO: 40 - protein, 264 aa, Mus musculus
MVWLPRVPCVAAVILLLTVLSPPVALVRDSRPWFLEYCKSECHFYNGTQRVRFLKRYFYNLEENLRFDSDVGEFRAVTELGRPDAENWNSQPEILEQKRAAVDTYCRHNYEIFDNFLVRRRVEPTVTVYPTKTQPLEHHNLLVCSVSDFYPGNIEVRWFRNGKEEKTGIVSTGLVRNGDWTFQTLVMLETVPQSGEVYTCQVEHPSLTDPVTVEWKAQSTSAQNKMLSGVGGFVLGLLFLGAGLFIYFRNQKGQSGLQPTGLLS

SEQ ID NO: 41 - DNA, 888 bp, Mus musculus
tgagtcctggtgactgccattacctgtgccttagagatggctctgcagatccccagcctcctcctctcggctgctgtggtggtgctgatggtgctgagcagcccagggactgagggcggagactccgaaaggcatttcgtgcaccagttcaagggcgagtgctacttcaccaacgggacgcagcgcatacggctcgtgaccagatacatctacaaccgggaggagtacctgcgcttcgacagcgacgtgggcgagtaccgcgcggtgaccgagctggggcggcactcagccgagtactacaataagcagtacctggagcgaacgcgggccgagctggacacggcgtgcagacacaactacgaggagacggaggtccccacctccctgcggcggcttgaacagcccaatgtcgccatctccctgtccaggacagaggccctcaaccaccacaacactctggtctgttcggtgacagatttctacccagccaagatcaaagtgcgctggttcaggaatggccaggaggagacagtgggggtctcatccacacagcttattaggaatggggactggaccttccaggtcctggtcatgctggagatgacccctcatcagggagaggtctacacctgccatgtggagcatcccagcctgaagagccccatcactgtggagtggagggcacagtccgagtctgcccggagcaagatgttgagcggcatcgggggctgcgtgcttggggtgatcttcctcgggctcggccttttcatccgtcacaggagtcagaaaggacctcgaggccctcctccagcagggctcctgcagtgactcagagtgttttgactcagttgactgtctcagactgtaaggcctgcatgtctctgcttc

SEQ ID NO: 42 - protein, 263 aa, Mus musculus
MALQIPSLLLSAAVVVLMVLSSPGTEGGDSERHFVHQFKGECYFTNGTQRIRLVTRYIYNREEYLRFDSDVGEYRAVTELGRHSAEYYNKQYLERTRAELDTACRHNYEETEVPTSLRRLEQPNVAISLSRTEALNHHNTLVCSVTDFYPAKIKVRWFRNGQEETVGVSSTQLIRNGDWTFQVLVMLEMTPHQGEVYTCHVEHPSLKSPITVEWRAQSESARSKMLSGIGGCVLGVIFLGLGLFIRHRSQKGPRGPPPAGLLQ

SEQ ID NO: 43 - DNA, 1173 bp, Mus musculus
gacagaactttgctttctgaagggggcacagcaggtgtgagtcctggtgactgccattacctgtgccttagagatggctctgcagatccccagcctcctcctctcggctgctgtggtggtgctgatggtgctgagcagcccagggactgagggcggagactccgaaaggcatttcgtgtaccagttcatgggcgagtgctacttcaccaacgggacgcagcgcatacgatatgtgaccagatacatctacaaccgggaggagtacgtgcgctacgacagcgacgtgggcgagcaccgcgcggtgaccgagctggggcggccagacgccgagtactggaacagccagccggagatcctggagcgaacgcgggccgagctggacacggtgtgcagacacaactacgaggggccggagacccacacctccctgcggcggcttgaacagcccaatgtcgtcatctccctgtccaggacagaggccctcaaccaccacaacactctggtctgctcagtgacagatttctaccccacccagatcaaagtgcgctggttccggaatggccaggaggagacggtgggggtctcatccacacagcttattaggaatggggactggaccttccaggtcctggtcatgctggagatgacccctcggcggggagaggtctacacctgtcacgtggagcatcccagcctgaagagccccatcactgtggagtggagggcacagtctgagtctgcctggagcaaaatgttgagcggcatcgggggctgcgtgcttggggtgatcttcctcgggcttggccttttcatccgtcacaggagtcagaaaggacctcgaggccctcctccagcagggctcctgcagtgactcagagtgttttgactcagttgactgtctcagactgtaagacctgaatgtctctgctccgaattcctgactgccagtccacctgccactccgactcagagtctagcatggtactattgtatccaccacctcagctcttgtgatctggagtcccccagtctctgtctgtagctctgctccttggtgattccagagactccatctgtgtacagccgcaccccaggctttctgtcttcgatctgtagtaaaccaatgtatgcttatccccacctagattacaaataaacgagactcagactctg

SEQ ID NO: 44 - protein, 265 aa, Mus musculus
MALQIPSLLLSAAVVVLMVLSSPGTEGGDSERHFVYQFMGECYFTNGTQRIRYVTRYIYNREEYVRYDSDVGEHRAVTELGRPDAEYWNSQPEILERTRAELDTVCRHNYEGPETHTSLRRLEQPNVVISLSRTEALNHHNTLVCSVTDFYPTQIKVRWFRNGQEETVGVSSTQLIRNGDWTFQVLVMLEMTPRRGEVYTCHVEHPSLKSPITVEWRAQSESAWSKMLSGIGGCVLGVIFLGLGLFIRHRSQKGPRGPPPAGLLQ

SEQ ID NO: 45 - DNA, 1204 bp, Mus musculus
cggacgcgtgggcagaactttgctttctgaagggggcacagcaggtgtgagtcctggtgactgccattacctgtgccttagagatggctctgcagatccccagcctcctcctctcggctgctgtggtggtgctgatggtgctgagcagcccagggactgagggcggagactccgaaaggcatttcgtgcaccagttcaagggcgagtgctacttcaccaacgggacgcagcgcatacggctcgtgaccagatacatctacaaccgggaggagtacctgcgcttcgacagcgacgtgggcgagtaccgcgcggtgaccgagctggggcggcactcagccgagtactacaataagcagtacctggagcgaacgcgggccgagctggacacggcgtgcagacacaactacgaggagacggaggtccccacctccctgcggcggcttgaacagcccaatgtcgccatctccctgtccaggacagaggccctcaaccaccacaacactctggtctgttcggtgacagatttctacccagccaagatcaaagtgcgctggttcaggaatggccaggaggagacagtgggggtctcatccacacagcttattaggaatggggactggaccttccaggtcctggtcatgctggagatgacccctcatcagggagaggtctacacctgccatgtggagcatcccagcctgaagagccccatcactgtggagtggagggcacagtccgagtctgcccggagcaagatgttgagcggcatcgggggctgcgtgcttggggtgatcttcctcgggctcggccttttcatccgtcacaggagtcagaaaggacctcgaggccctcctccagcagggctcctgcagtgactcagagtgttttgactcagttgactgtctcagactgtaaggcctgcatgtctctgcttcgaattcctgactgccagtctacctgccactccgactcagagtctagcatggcactattgtatccaccacctcagctcttgtgatctggagtcccccagtctctgtctgtagctctgctccttggtgattccagagactccatctgtgtacagcagcaccccaggctttctgtcttcgctctgtagtaaaccaatgtatgcttatccccacctagattacaaataaacgagacccagactctgaaaaaaaaaaaaaaaaaaaaaaaaaaa

SEQ ID NO: 46 - protein, 263 aa, Mus musculus; identical to SEQ ID NO: 42

SEQ ID NO: 47 - DNA, 1162 bp, Mus musculus
ggcacagcaggtgtgagtcctggtgactgccattacctgtgccttagagatggctctgcagatccccagcctcctcctctcggctgctgtggtggtgctgatggtgctgagcagcccagggactgagggcggagactccgaaaggcatttcgtgttccagttcaagggcgagtgctacttcaccaacgggacgcagcgcatacgatctgtggacagatacatctacaaccgggaggagtacctgcgcttcgacagcgacgtgggcgagtaccgcgcggtgaccgagctggggcggccagaccccgagtactacaataagcagtacctggagcaaacgcgggccgagctggacacggtgtgcagacacaactacgagggggtggagacccacacctccctgcggcggcttgaacagcccaatgtcgtcatctccctgtccaggacagaggccctcaaccaccacaacactctggtctgctcagtgacagatttctacccagccaagatcaaagtgcgctggttccggaatggccaggaggagacggtgggggtctcatccacacagcttattaggaatggggactggaccttccaggtcctggtcatgctggagatgacccctcggcggggagaggtctacacctgccacgtggagcatccgagcctgaagagccccatcactgtggagtggagggcacagtctgagtctgcccggagcaagatgttgagcggcatcgggggctgcgtgcttggggtgatctttctcgggcttggccttttcatccgtcacaggagtcagaaaggacctcgaggccctcctcctgcagggctcctgcagtgactcagagtgttttgactcagttgactatctcagactgtaaggcctgcatgtctctgctctgaattcctgactgccagtctacctgccactctatgactcagagtctagcatggtactattgtatccaccacatcagctcttatgatctggagtcccccagtctctgtctgtagctctgctccttggtgattccagagactccatctgtgtacagcagcaccccaggctttctgtcttcgctctgtagtaaaccaatgtatgcttatccccacctagactacaaataaacgagacccagactctgaaaaaaaaaaaaaaaaa

SEQ ID NO: 48 - protein, 263 aa, Mus musculus
MALQIPSLLLSAAVVVLMVLSSPGTEGGDSERHFVFQFKGECYFTNGTQRIRSVDRYIYNREEYLRFDSDVGEYRAVTELGRPDPEYYNKQYLEQTRAELDTVCRHNYEGVETHTSLRRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWRAQSESARSKMLSGIGGCVLGVIFLGLGLFIRHRSQKGPRGPPPAGLLQ

SEQ ID NO: 49 - DNA, 1172 bp, Mus musculus
acagaactttgctttctgaagggggcacagcaggtgtgagtcctggtgactgccattacctgtgccttagagatggctctgcagatccccagcctcctcctctcggctgctgtggtggtgctgatggtgctgagcagcccagggactgagggcggagactccgaaaggcatttcgtgtaccagttcatgggcgagtgctacttcaccaacgggacgcagcgcatacgatatgtgaccagatacatctacaaccgggaggagtacgtgcgctacgacagcgacgtgggcgagcaccgcgcggtgaccgagctggggcggccagacgccgagtactggaacagccagccggagatcctggagcgaacgcgggccgagctggacacggtgtgcagacacaactacgaggggccggagacccacacctccctgcggcggcttgaacagcccaatgtcgtcatctccctgtccaggacagaggccctcaaccaccacaacactctggtctgctcagtgacagatttctacccagccaagatcaaagtgcgctggttccggaatggccaggaggagacggtgggggtctcatccacacagcttattaggaatggggactggaccttccaggtcctggtcatgctggagatgacccctcggcggggagaggtctacacctgtcacgtggagcatcccagcctgaagagccccatcactgtggagtggagggcacagtctgagtctgcctggagcaagatgttgagcggcatcgggggctgcgtgcttggggtgatcttcctcgggcttggccttttcatccgtcacaggagtcagaaaggacctcgaggccctcctccagcagggctcctgcagtgactcagagtgttttgactcagttgactgtctcagactgtaagacctgaatgtctctgctccgaattcctgactgccagtccacctgccactccgactcagagtctagcatggtactattgtatccaccacctcagctcttgtgatctggagtcccccagtctctgtctgtagctctgctccttggtgattccagagactccatctgtgtacagccgcaccccaggctttctgtcttcgatctgtagtaaaccaatgtatgcttatccccacctagattacaaataaacgagactcagactctg

SEQ ID NO: 50 - DNA, 798 bp, Mus musculus
atggctctgcagatccccagcctcctcctctcggctgctgtggtggtgctgatggtgctgagcagcccaaggactgagggcggaaactccgaaaggcatttcgtggcccagttgaagggcgagtgctacttcaccaacgggacgcagcgcatacgatctgtgaacagatacatctacaaccgggaggagtgggtgcgcttcgacagcgacgtgggcgagtaccgcgcggtgaccgagctggggcggccagacgccgagtactggaacagccagccggagatcctggagcgaacgagggccgaggtggacacggtgtgcagacacaactacgagggggtggagacccacacctccctgcggcggcttgaacagcccaatgtcgccatctccctgtccaggacagaggccctcaaccaccacaacactctggtctgctcggtgacagatttctacccagccaagatcaaagtgcgctggttcaggaatggccaggaggagacagtgggggtctcatccacacagcttattaggaatggggactggaccttccaggtcctggtcatgctggagatgacccctcatcagggagaggtctacacctgccatgtggagcatcccagcctgaagagccccatcactgtggagtggagggcacagtccgagtctgcccggagcaagatgttgagtggcatcgggggctgcgtgcttggggtgatcttcctcggtcttggccttttcatccgtcacaggagtcagaaaggacctcgaggccctcctccagcagggctcctgcagtga

SEQ ID NO: 51 - protein, 265 aa, Mus musculus
MALQIPSLLLSAAVVVLMVLSSPRTEGGNSERHFVAQLKGECYFTNGTQRIRSVNRYIYNREEWVRFDSDVGEYRAVTELGRPDAEYWNSQPEILERTRAEVDTVCRHNYEGVETHTSLRRLEQPNVAISLSRTEALNHHNTLVCSVTDFYPAKIKVRWFRNGQEETVGVSSTQLIRNGDWTFQVLVMLEMTPHQGEVYTCHVEHPSLKSPITVEWRAQSESARSKMLSGIGGCVLGVIFLGLGLFIRHRSQKGPRGPPPAGLLQ

SEQ ID NO: 52 - DNA, 792 bp, Mus musculus
atggctctgcagatccccagcctcctcctcttggctgctgtggtggtgctgatggtgctgagcagcccagggactgagggcggagactccgaaaggcatttcttggtccagttccagcccttctgctacttcaccaacgggacgcagcgcatacgatatgtgaccagatacatctacaaccgggaggagtacctgcgcttcgacagcgacgtgggcgagtaccgcgcggtgaccgagctggggcggccagacgccgagtactacaataagcagtacctggagcgaacgcgggccgagctggacacggtgtgcagatacaactacgaggagacggaggtccccacctccctgcggcggcttgaacagcccaatgtcgtcatctccctgtccaggacagaggccctcaaccaccacaacactctggtctgctcagtgacagatttctacccagccaagatcaaagtgcgctggttccggaatggccaggaggagacggtgggggtctcatccacacagcttattaggaatggggactggaccttccaggtcctggtcatgctggagatgacccctcggcggggagaggtctacacctgccatgtggagcatcccagcctgaagagtcccatcaccgtggagtggcgggcacagtccgagtctgcccggagcaagatgttgagcggcatcgggggctgcgtgcttggggtgatcttcctcgggctcggccttttcatccgtcacaggagtcagaaaggacctcgaggccctcctccagcagggctcctgcagtga

SEQ ID NO: 53 - protein, 263 aa, Mus musculus
MALQIPSLLLLAAVVVLMVLSSPGTEGGDSERHFLVQFQPFCYFTNGTQRIRYVTRYIYNREEYLRFDSDVGEYRAVTELGRPDAEYYNKQYLERTRAELDTVCRYNYEETEVPTSLRRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWRAQSESARSKMLSGIGGCVLGVIFLGLGLFIRHRSQKGPRGPPPAGLLQ

SEQ ID NO: 54 - DNA, 1433 bp, Mus musculus
gggggtacaaggtcacagagccccctctgccttcttcctctcctagaggttaaaagagagcaaggacaacactcttgacgctgggacctaggcggccttccggggaagatgaagatcctcctgctactgctgaccttgtctctggcctccaggacaaaggcaggggagatcatcgggggacatgaagtcaagccccactctcgaccctacatggccttactttcgatcaaggatcagcagcctgaggcgatatgtgggggcttccttattcgagaggactttgtgctgactgctgctcactgtgaaggaagtataataaatgtcactttgggggcccacaacatcaaagaacaggagaagacccagcaagtcatccctatggtaaaatgcattccccacccagactataatcctaagacattctccaatgacatcatgctgctaaagctgaagagtaaggccaagaggactagagctgtgaggcccctcaacctgcccaggcgcaatgtcaatgtgaagccaggagatgtgtgctatgtggctggttggggaaggatggccccaatgggcaaatactcaaacacgctacaagaggttgagctgacagtacagaaggatcgggagtgtgagtcctactttaaaaatcgttacaacaaaaccaatcagatatgtgcgggggacccaaagaccaaacgtgcttcctttcggggggattctggaggcccgcttgtgtgtaaaaaagtggctgcaggcatagtttcctatggatataaggatggttcacctccacgtgctttcaccaaagtctcgagtttcttatcctggataaagaaaacaatgaaaagcagctaactacagaagcaacatggatcctgctctgattacccatcgtccctagagctgagtccaggattgctctaggacaggtggcaggatctgaataaaggactgcaaagactggcttcatgtccattcacaaggaccagctctgtccttggcaggccaatggaacacctcttctgccaccatgctgtgacaacccaactgacatcttcctatggaagtttgccctctccacaaaagaagtagaatgtttgcattggagctgggcatgctctgcttcccctcagtgccccgagaatgttatctaatgctagtcatcattaatagctccctacagaactttcatacagttgcacccaagttgctgatgtgttctctagaatagagcaagaaatagtaaacagaattccttttgcctctctgtactattttcccccaaataccaagatttgtatgttttataaagctaatttccttatcaaatgacatcttttaatttttacattaatggcttattttcaaggtacaacctgatttttttatggacaaaaatgatgtaaaatcaaataaaactaattaatatatccatt

SEQ ID NO: 55 - DNA, 2696 bp, Mus musculus
ggcacgaggatttctgactctcagctggtttttgttccctgtgcttgtaggatggaaaaacagatgtaaaatccctcatggcgaagttcaacacggggagtaacccgacagaggaggccgccaccagcagcaggcccttcaaagttgcaggacaaagctcgccttctggaatacaatcaagaaagaatttatttgacaaccaaggaaatgccagcccgcccgcaggacccagcagcatgcctaaatttgggaccacaaaaccacccttggcagcgaaacctacctacgaggagaagcctgagaaggaacctaaacccccgtttttaaaacctactggagggagcccaagatttggaacacagccaaactcagtttctagagaccctgaggtcaaagtggggttcctgaaacctgtaagccccaaacccaccagcttgaccaaagaagattccaaacctgtggttttacggccacctgggaataaacttcacaatttgaaccaagagtctgacttaaagacgccaggaccgaagccagggcctgctcccccagtcccagaaaacgaactgaagccagggttctcaaaggtggctggagctaagagcaagttcatgccagcggcacaagatactgactccaaaccccgatttcccagacacacctttggccagaagccatccctaagcacagaggactcccaggaagagaacacttctaagaacgtgcctgtccagaaaggatccccagtgcagttgggcgccaagtccaagggcgcccctttcaaaccacctaaggaagacccagaggataaagaccatggagcaccaagttcaccctttcctggagtggttctgaaacctgctgcgagcagagggagcccgggtctctccaaaaactttgaagaaaaaaaagaagataggaaaacagaccttgcaaagaacatctttctgaacaaactgaatcaagaagagccggccagattccctaaggccccttccaagctgacagcggggacaccgtggggccaaagtcaggagaaagaaggagacaagaattctgcaaccccgaagcagaaggccctgccacccctgtccgtcttgggcccgcctccacccaagcccaacagaccacccaacgttgacctgaccagattccgaaaggctgactctgcaaacagtgccaccaaaagccagacaccttactcgacaacttccctgccaccacctccgcctacacatccagccagccagccacctctgccagcgtctcacccagcgcatccgccagtccccagcctgccccccagaaacatcaaacctccccttgacctgaaacatcctataaatgacgaaaatcaagatggtgtcatgcactctgatggtactggaaatctagaagaggaacaagagagtgaaggggaaacatacgaagacatagattcatctaaggaacgagacaagaaacgggaaaaagaagaaaagaagaggttggaactagaacgaaaggagcagaaggagagagagaagaaagagcaagaactgaagaagaaattcaagctcacgggccccattcaagtcatccaccatgcaaaagcctgctgcgatgtcaaaggaggaaagaatgagctgagcttcaaacaaggagaggacattgagattatccgcatcacagacaacccagaaggaaaatggctgggcaggactgcaagggggtcatatggttatattaaaacaactgccgtggagattgactatgattcgctgaaacggaagaagaactccctcaatgctgtcccaccaagacttgttgaagatgaccaggatgtctatgatgatgtggctgagcaggatgcccctaacagccacggtcagagcggaagtggagggatgttccccccgccaccaacagacgacgagatctatgacggaatagaggaggaagatgatgacgatggttccgtgccacaggttgacgagaagactaacgcatggtcctgggggattttgaagatgttaaagggaaaagatgacagaaagaaaagcattcgagagaaacctaaagtctctgaatcagacaataatgaaggttcatctctcccttctcagcataagcaactggatgtgggagaagaagtttacgatgatgtggatgcctcagacttccctcctccacctgcagagatgagtcaaggaatgagtgttggaagggcaaaaacggaagagaaagatcccaagaagctaaaaaagcaagaaaaggaagaaaaagacctcaggaaaaaatttaagtacgacggtgaaattcgagttctatattcaactaaagttgcgtcctccttaacctctaaaaagtggggagcgagagatctgcagataaaacctggggagtcactcgaagttatacaaagcacagatgacaccaaagttctctgcaggaatgaagagggcaaatatggttatgtccttcggagttacctggtggacaatgatggagaaatctatgacgacatcgctgatggttgcatctatgacaatgactagcactctgctctgttcattccactgtgcctcttagtggccaacatgaattttaattagaatgttcttgggtattatagaaaaaaatgtgaacatacaaaatgaaatgtcaatctctataaagttttgaaattttaaacctgaaaaaaaaaaaaaaaaaaa

SEQ ID NO: 56 - protein, 819 aa, Mus musculus
MAKFNTGSNPTEEAATSSRPFKVAGQSSPSGIQSRKNLFDNQGNASPPAGPSSMPKFGTTKPPLAAKPTYEEKPEKEPKPPFLKPTGGSPRFGTQPNSVSRDPEVKVGFLKPVSPKPTSLTKEDSKPVVLRPPGNKLHNLNQESDLKTPGPKPGPAPPVPENELKPGFSKVAGAKSKFMPAAQDTDSKPRFPRHTFGQKPSLSTEDSQEENTSKNVPVQKGSPVQLGAKSKGAPFKPPKEDPEDKDHGAPSSPFPGVVLKPAASRGSPGLSKNFEEKKEDRKTDLAKNIFLNKLNQEEPARFPKAPSKLTAGTPWGQSQEKEGDKNSATPKQKALPPLSVLGPPPPKPNRPPNVDLTRFRKADSANSATKSQTPYSTTSLPPPPPTHPASQPPLPASHPAHPPVPSLPPRNIKPPLDLKHPINDENQDGVMHSDGTGNLEEEQESEGETYEDIDSSKERDKKREKEEKKRLELERKEQKEREKKEQELKKKFKLTGPIQVIHHAKACCDVKGGKNELSFKQGEDIEIIRITDNPEGKWLGRTARGSYGYIKTTAVEIDYDSLKRKKNSLNAVPPRLVEDDQDVYDDVAEQDAPNSHGQSGSGGMFPPPPTDDEIYDGIEEEDDDDGSVPQVDEKTNAWSWGILKMLKGKDDRKKSIREKPKVSESDNNEGSSLPSQHKQLDVGEEVYDDVDASDFPPPPAEMSQGMSVGRAKTEEKDPKKLKKQEKEEKDLRKKFKYDGEIRVLYSTKVASSLTSKKWGARDLQIKPGESLEVIQSTDDTKVLCRNEEGKYGYVLRSYLVDNDGEIYDDIADGCIYDND

SEQ ID NO: 57 - DNA, 2972 bp, Mus musculus
gagcctgggccagtcttcgggagcccaatggctaactatgaatttagccaggtgtctggggacagacctggctgccgcctctctaggaaagcccagatcggtctcggagtgggtctcctggtcctgatcgccttggtagtagggatcgtggtcatacttctgaggccgcgctcactcctggtgtggactggagagcctaccacgaagcacttttctgacatcttcctgggacgctgcctcatctacactcagatcctccggccggagatgagagatcagaactgccaggagatactgagtacattcaaaggagcatttgtttccaagaacccttgcaacatcacaagagaagactacgccccacttgttaaattggtcactcaaaccataccatgtaacaagactctcttttggagcaaatccaaacacctggcccatcaatatacttggatccagggaaagatgttcaccctggaggacaccctgctgggctacattgctgatgatctcaggtggtgtggagaccctagtacttctgatatgaactatgtctcttgcccacattggagtgaaaactgtcccaacaaccctattactgtgttctggaaagtgatttcccaaaagtttgcagaagatgcctgtggtgtggtccaagtgatgctcaatgggtccctccgtgagccattttacaaaaacagcacctttggaagtgtggaagtctttagtttggacccaaataaggttcataaactacaggcctgggtgatgcacgacatcgaaggagcttccagtaacgcatgttcaagctcctccttaaatgagctgaagatgattgtgcagaaaaggaatatgatatttgcctgcgtggataactacaggcctgccaggtttcttcagtgtgtgaagaaccctgagcacccatcgtgtagacttaatacgtgaaggatctggatcttagatcacctgtagcctggactgagatgaaggggctcagaagcaacactggtggaaagctgaaactgtcagggagaagcctctactacagtgttaacaccagagatggaagaacttcccaattctctgtgtactaccaacattcaagaaaaattactccataaaccagagttaaacttctatattgttatattagtctaactttctcatgtggtgcttctgtattgtttatatattgcttacatccttttattcctcttttaatgatctctcttttctctctctctctctctctctctctctcaatgaggctgagaatccaacctgagaactttcatacatggtggataagcctatataccactgagctaaatcctcagcacagctgataacatcatttttgctgaaaaatggccaatcaaacttcccattagacaaagaaagtcaaatgtcaagtatatcgaaatgaatgaccctttttttttttttatgttttttcattcttcccacagatattcacatggtaaacctgaggtcatagggtcattatagggaaggtgctgtgtgggaactacccacgtgccctgtgctttaatctttaactcaacacatccctgataactttgagcattcttttcttttcttttctttccttttcttttcttttcttttctttttcttttttttaaacagaagaaaatggagtttagagagataattgattttcctaaagtcacattcttaagatcatggttacatctagaattaaatcagttctgtcaaagtccacaacatatgatagtttttttcttttccgagcttttattgccaaattggtagtagcctgtgttgtcgtctagcaaatgctattgcattagattgaatataatagaatctgaaatgtgatctttatggaagatcacaaaactaattacttgcaccctctttctcttccacttcattcttggtgccttttataggcagtcaagagttaacatatatttgttgctaatattggcaatttatgttttctatcctttttttcttagatcagtcttgctcagaatcactggttttgttatgttgttgttgttgctattgttattgttcttgtctttttcaaagctcaaatctttaactttcctcatttatttatttaattgagctttagtctttatttttagaaacatagaaatttcttctagctatctggaatatcttcagttttttgaggcaaatgcttagaccattgatatttcagtctgttttctacacatgtactttaggattctaggtttctccctgagccctgctttcgatgtaacactgaatttctgtatgtctttactggttagttactttgatagtttgtatatgcttgacccagtgagtggcaggattagaaggtatggccttgctggaataggtgtgccactgtgggtgtagtcttaagacccttaccctagctgcctggaggccactattccactaacagccttcaaatgaaaatataaaactctcagctctgcctgtgccatgcctgcctggatgctgccatgctcccaccttgatgataatggactgaacctctgaacctgtaagccagccccaatttgttgtccttataaaagacttgctttggtcatggtatctgttcacagcagaaagaacctaactaagacagttaccattcagttcaaaataattcttgattttattgttatttagacatgtgatatttacttttcaacatctggagaattgtttaggttttttttgtgtgtgtgtctttagtaggtattaataaactaaattgagacaactccaaagtatttgcttggtttattgaaaatagcataaacagagccaggtgcttctgtattcatgttttgacaccagtttgtgattgttgaacaatatatcttatttctaaacttttaaaaagctacttaaagacattaacttgctcccttacaatatgttgatggttcagtccattaaatgcttactttgtaagt

SEQ ID NO: 58 - protein, 304 aa, Mus musculus
MANYEFSQVSGDRPGCRLSRKAQIGLGVGLLVLIALVVGIVVILLRPRSLLVWTGEPTTKHFSDIFLGRCLIYTQILRPEMRDQNCQEILSTFKGAFVSKNPCNITREDYAPLVKLVTQTIPCNKTLFWSKSKHLAHQYTWIQGKMFTLEDTLLGYIADDLRWCGDPSTSDMNYVSCPHWSENCPNNPITVFWKVISQKFAEDACGVVQVMLNGSLREPFYKNSTFGSVEVFSLDPNKVHKLQAWVMHDIEGASSNACSSSSLNELKMIVQKRNMIFACVDNYRPARFLQCVKNPEHPSCRLNT

SEQ ID NO: 59 - DNA, 1896 bp, Mus musculus
agaagaaagttaagatcggcccggcctctgcaacgcgcgccctgctaccatcctcaagtgcggctcttgcaacagctgccaccgccggcgccagcgggacctaatccaaccaggccgaggtcacagcggaggagcgatgtgccaccgcagccctggcaggagcctcaggccgccatcgccgctgctgctgctgctgccattgctgctgcagcctccctgggcggctggcgctgccagccagagcgacccaacaggagtccactgtgcaacagcatactggtcccctgaatcgaagaaagtggaaatcaagacggtactggacaagaatggggacgcctacggctactacaatgactccattaaaaccacaggctggggcatcctggagatcagagctgggtacggctctcaggtcctgtccaatgagatcatcatgtttttggctggctacctcgagggctacctcactgctctacacatgtacgaccacttcacgaacctgtacccacagctgttcaagaatccttccattgtgaaaaaagtgcaagacttcatggagaagcaagaaatgtggactaggcaaaacatcaaagcgcagaaggatgacccattctggagacacaccggctatgtggtgacccagctcgacgggctgtatcttggagctcagaaaagggcttcagaagagaaaataaagcccatgacaatgttccaaattcagttcctgaacgccgttggtgacctgttggaccttattccctccctatctcccacaaaaagcagcagcatgatgaagtttaagatttgggaaatgggccactgttctgctcttattaaggtccttcctggatttgaaaacatctattttgcccactcaagttggtacacatatgcagccatgctcaggatctataaacattgggacttcaacatcaaggacaaatacaccctcagcaaacggctgtctttcagcagttatccagggttcttggagtctctggatgacttttatattctcagcagtggcctgatattgctgcagaccacaaacagtgtgtacaataaaaccctgctgaagcaggtggtgcccaagactctcctggcctggcaaagagtccgtgtggccaacatgatggccgagggtggtaaggagtgggcacagatattttcaaagcacaactctggtacctacaacaaccagtacatggtcctggacctgaagaaggtgaccatcaataggagcctggacaaaggcactctctatattgtggagcaaatccctacatatgtagaatactccgatcaaaccaatgtcctaagaaaaggatactgggcttcctataatatccctttccacaaaacaatctacaactggagtggctaccctctgctggttcacaagctgggcttggactactcttatgacttagctccacgtgccaaaatcttcaggcgtgaccaagggaacgtgacggacatggcctccatgaaatatatcatgcgatacaacaattataaagaggacccctacagcaagggggatccctgcagtaccatctgctgtcgtgaggacctgaatggagccagtccaagccctggaggatgctatgacaccaaggtggcagacatctttcttgcatctcagtataaagcctatgccattagtggtcccacggtacaagatggactcccgccttttaactggaatcggttcaatgacacgctccatcgggggatgccagaagtctttgactttaattttgttaccatgaaaccaattttgtcataggacaaaaagtgagacatagagatgacagaaagctgcgaataggacaccaaaggcactcttagctgtattttcccatctgtactcttaataaaaaatattaaattccc

SEQ ID NO: 60 - protein, 550 aa, Mus musculus
MCHRSPGRSLRPPSPLLLLLPLLLQPPWAAGAASQSDPTGVHCATAYWSPESKKVEIKTVLDKNGDAYGYYNDSIKTTGWGILEIRAGYGSQVLSNEIIMFLAGYLEGYLTALHMYDHFTNLYPQLFKNPSIVKKVQDFMEKQEMWTRQNIKAQKDDPFWRHTGYVVTQLDGLYLGAQKRASEEKIKPMTMFQIQFLNAVGDLLDLIPSLSPTKSSSMMKFKIWEMGHCSALIKVLPGFENIYFAHSSWYTYAAMLRIYKHWDFNIKDKYTLSKRLSFSSYPGFLESLDDFYILSSGLILLQTTNSVYNKTLLKQVVPKTLLAWQRVRVANMMAEGGKEWAQIFSKHNSGTYNNQYMVLDLKKVTINRSLDKGTLYIVEQIPTYVEYSDQTNVLRKGYWASYNIPFHKTIYNWSGYPLLVHKLGLDYSYDLAPRAKIFRRDQGNVTDMASMKYIMRYNNYKEDPYSKGDPCSTICCREDLNGASPSPGGCYDTKVADIFLASQYKAYAISGPTVQDGLPPFNWNRFNDTLHRGMPEVFDFNFVTMKPILS

SEQ ID NO: 61 - DNA, 1053 bp, Mus musculus
agggttcttggagtctctggatgacttttatattctcagcagtggcctgatattgctgcagaccaccaacagtgtgtacaataagaccctgctgaagcaggtggtgcccaagactctcctggcctggcaaagagtccgtgtggccaacatgatggccgagggtggtaaggagtgggcacagatattttcaaagcacaactctggtaagtggttctgtacagcagtcctaaggcctatgtgcctatgtctagccttacagttgttaatcagttgacgttattaatcacttaaccagttgaggcatatccctgtcgccttgtgtcttccaacatttcaggtacctacaacaaccagtacatggtcctggacctgaagaaggtgaccatcaataggagcctggacaaaggcactctctatattgtggagcaaatccctacatatgtagaatactccgatcaaaccaatgtcctaagaaaaggatactgggcttcctataatatccctttccacaaaacaatctacaactggagtggctaccctctgctggttcacaagctgggcttggactactcttatgacttagctccacgtgccaaaatcttcaggcgtgaccaagggaacgtgacggacatggcctccatgaaatatatcatgcgatacaacaattataaagaggacccctacagcaagggggatccctgcagtaccatctgctgtcgtgaggacctgaatggagccagtccaagccctggaggatgctatgacaccaaggtggcagacatctttcttgcatctcagtataaagcctatgccattagtggtcccacggtacaagatggactcccgccttttaactggaatcggttcaatgaaacgctccatcgggggatgccagaagtctttgactttaattttgttaccatgaaaccaattttgtcataggacaaaaagtgagacatagagatgacagaaagctgcgaataggacaccaaaggcactcttagctgtattttcccatctgtactcttaataaaaaatattaaattcccatct

SEQ ID NO: 62 - protein, 194 aa, Mus musculus
MVLDLKKVTINRSLDKGTLYIVEQIPTYVEYSDQTNVLRKGYWASYNIPFHKTIYNWSGYPLLVHKLGLDYSYDLAPRAKIFRRDQGNVTDMASMKYIMRYNNYKEDPYSKGDPCSTICCREDLNGASPSPGGCYDTKVADIFLASQYKAYAISGPTVQDGLPPFNWNRFNETLHRGMPEVFDFNFVTMKPILS

SEQ ID NO: 63 - DNA, 2314 bp, Mus musculus
agacacaaagctaaggtctgaaggtgttgagctcctgaggtcagacaggctggcgcaagaagccctggttcctgacggatgagggacctgagaatacagaaggccacagaatccgacctacggggaggcatgcgacccaccgctgcctcggtttccttatcctctgctacagggaccaaggccacctttaactgattccttagccctgagccccaccctccaggaggcgaccagagaaagcagcacagccccggcacttcccaaaagcgcggcagagaactttcccggcccggcccacaggtggctggagggtcagaccagacggaaaggacccgcctctcctagcctggggctcagcagggcgaaggttggttcccaaggccacgtgaagcgcccaagcaggcaagactaatgtcagaagaaagttaagatcggcccggcctctgcaacgcagcgccctgctaccatcctcaagtgcggctcttgcaacagctgccaccgccggcgccagcgggacctaatccaaccaggccgaggtcacagcagaggagcgatgtgccaccgcagccctggcaggagcctcaggccgccatcgccgctgctgctgctgctgccattgctgctgcagcctccctgggcggccggtgctgccagccagagcgacccaacaggagtccactgtgcaacagcatactggtcccctgaatccaagaaagtggaaatcaagacggtactggacaagaatggggacgcctacggctactacaatgactccattaaaaccacaggctggggcatcctggagatcagagctgggtacggctctcaggtcctgtccaatgagatcatcatgtttctggctggctacctcgagggctacctcactgctctgcatttatctctgagtaactttattcatttgctgccttccccgcacccccagttcattaaaagcattcctgacttccttcagaagcaagaaatgtggactaggcaaaacatcaaagcgcagaaggatgacccattctggagacacaccggctatgtggtgacccagctcgacgggctgtatcttggagctcagaaaagggcttcagaagagaaaataaagcccatgacaatgttccaaattcagttcctgaacgccgttggtgacctgttggaccttattccctccctatctcccacaaaaagcagcagcatgatgaagtttaagatttgggaaatgggccactgttccgctcttattaaggtccttcctggatttgaaaacatctattttgcccactcaagctggtacacatatgcagccatgctcaggatctataaacattgggacttcaacatcaaggacaaatacaccctcagcaaacggctgtctttcagcagttatccagggttcttggagtctctggatgacttttatattctcagcagtggcctgatattgctgcagaccaccaacagtgtgtacaataagaccctgctgaagcaggtggtgcccaagactctcctggcctggcaaagagtccgtgtggccaacatgatggccgagggtggtaaggagtgggcacagatattttcaaagcacaactctggtacctacaacaaccagtacatggtcctggacctgaagaaggtgaccatcaataggagcctggacaaaggcactctctatattgtggagcaaatccctacatatgtagaatactccgatcaaaccaatgtcctaagaaaaggatactgggcttcctataatatccctttccacaaaacaatctacaactggagtggctaccctctgctggttcacaagctgggcttggactactcttatgacttagctccacgtgccaaaatcttcaggcgtgaccaagggaacgtgacggacatggcctccatgaaatatatcatgcgatacaacaattataaagaggacccctacagcaagggggatccctgcagtaccatctgctgtcgtgaggacctgaatggagccagtccaagccctggaggatgctatgacaccaaggtggcagacatctttcttgcatctcagtataaagcctatgccattagtggtcccacggtacaagatggactcccgccttttaactggaatcggttcaatgaaacgctccatcgggggatgccagaagtctttgactttaattttgttaccatgaaaccaattttgtcataggacaaaaagtgagacatagagatgacagaaagctgcgaataggacaccaaaggcactcttagctgtattttcccatctgtactcttaataaaaaatattaaattcccatct

SEQ ID NO: 64 - protein, 549 aa, Mus musculus
MCHRSPGRSLRPPSPLLLLLPLLLQPPWAAGAASQSDPTGVHCATAYWSPESKKVEIKTVLDKNGDAYGYYNDSIKTTGWGILEIRAGYGSQVLSNEIIMFLAGYLEGYLTALHLSLSNFIHLLPSPHPQFIKSIPDFLQKQEMWTRQNIKAQKDDPFWRHTGYVVTQLDGLYLGAQKRASEEKIKPMTMFQIQFLNAVGDLLDLIPSLSPTKSSSMMKFKIWEMGHCSALIKVLPGFENIYFAHSSWYTYAAMLRIYKHWDFNIKDKYTLSKRLSFSSYPGFLESLDDFYILSSGLILLQTTNSVYNKTLLKQVVPKTLLAWQRVRVANMMAEGGKEWAQIFSKHNSGTYNNQYMVLDLKKVTINRSLDKGTLYIVEQIPTYVEYSDQTNVLRKGYWASYNIPFHKTIYNWSGYPLLVHKLGLDYSYDLAPRAKIFRRDQGNVTDMASMKYIMRYNNYKEDPYSKGDPCSTICCREDLNGASPSPGGCYDTKVADIFLASQYKAYAISGPTVQDGLPPFNWNRFNETLHRGMPEVFDFNFVTMKPILS

SEQ ID NO: 65 - DNA, 526 bp, Mus musculus
ttcaataaacacacttgatttattttgtataaaagggttaagtttacaactaaacttttataaaaagtttagcataagtacgtcattacattttaatgcagaaataggttatttctgccactatacaagaaaactctaattaaagagtccacaaggttcactcaatatatatatatttatatataaatatatacacagcattcagtaggctcgtgtcaaaaaggccatttctgaccgaagacttcatttaagtaaccgaatactgggtccttctggtattcacgctccacctttgtgaaaaggcaaagtctgcttacattgggcaattccttgtgatttttttcttttttttttaacgttttcgctccccatggtgggaatagtatataaagaaaaccttccaactgtagaaagggcactgaaagtctctcataaataacggtatcacagtccaggccggagaacgacacctgggcaagacgatcatccttagttttccttccgctttctttttaaaaaggtccat

SEQ ID NO: 66 - DNA, 1268 bp, Mus musculus
attctgaaccgagcctggtgccgcgcagtcagctcagccccctgtggcggctccctcccggtcttcctcctacgagcagcatgaaagccttcagtccggtgaggtccgttaggaaaaacagcctgtcggaccacagcttgggcatctcccggagcaaaaccccggtggacgacccgatgagtctgctctacaacatgaacgactgctactccaagctcaaggaactggtgcccagcatcccccagaacaagaaggtgaccaagatggaaatcctgcagcacgtcatcgattacatcttggacctgcagatcgccctggactcgcatcccactatcgtcagcctgcatcaccagagacctggacagaaccaggcgtccaggacgccgctgaccaccctgaacacggacatcagcatcctgtccttgcaggcatctgaattcccttctgagcttatgtcgaatgatagcaaagtactctgtggctaaataaatggcatttggggacttttttttttctttttactttctctttttcttttgcacaagaagaagtctacaagatcttttaagacttttgttatcagccatttcaccaggagaacacgttgaatggacctttttaaaaagaaagcggaaggaaaactaaggatgatcgtcttgcccaggtgtcgttctccggcctggactgtgataccgttatttatgagagactttcagtgccctttctacagttggaaggttttctttatatactattcccaccatggggagcgaaaacgttaaaaaaaaaagaaaaaaatcacaaggaattgcccaatgtaagcagactttgccttttcacaaaggtggagcgtgaataccagaaggacccagtattcggttacttaaatgaagtcttcggtcagaaatggcctttttgacacgagcctactgaatgctgtgtatatatttatatataaatatatatatattgagtgaaccttgtggactctttaattagagttttcttgtatagtggcagaaataacctatttctgcattaaaatgtaatgacgtacttatgctaaactttttataaaagtttagttgtaaacttaacccttttatacaaaataaatcaagtgtgtttattgaatgttgattgcttgctttatttcagacaaccagtgctttgattttttttatgctatgttataactgaacccaaataaataccagttcaaatttatgtagactgtattaagattataataaaatgtgtctgacatc

SEQ ID NO: 67 - DNA, 1280 bp, Mus musculus
gcgcagtcagctcagccccctgtggcggctccctcccggtctctcctcctacgagcagcatgaaagccttcagtccggtgaggtccgttaggaaaaacagcctgtcggaccacagcttgggcatctcccggagcaaaaccccggtggacgacccgatgagtctgctctacaacatgaacgactgctactccaagctcaaggaactggtgcccagcatcccccagaacaagaaggtgaccaagatggaaatcctgcagcacgtcatcgattacatcttggacctgcagatcgccctggactcgcatcccactatcgtcagcctgcatcaccagagacctggacagaaccaggcgtccaggacgccgctgaccaccctgaacacggacatcagcatcctgtccttgcaggcatctgaattcccttctgagcttatgtcgaatgatagcaaagtactctgtggctaaataaatggcatttggggactttttcttttctttttactttctctttttcttttgcacaagaagaagtctacaagatcttttaagacttttgttatcagccatttcaccaggagaacacgttgaatggacctttttaaaaagaaagcggaaggaaaactaaggatgatcgtcttgcccaggtgtcttgttctccggcctggactgtgataccgttatttatgagagactttcagtgccctttctacagttggaaggttttctttatatactattcccaccatggggagcgaaaaggttaaaaaaaagaaaaaaatcacaaggaattgcccaatgtaagcagactttgccttttcacaaaggtggagcgtgaattccaggaggacccagtattcggttacttaaatgaagtcttcggtcagaaatggcctttttgacacgagcctactgaatgctgtgtatatatttatatataaatatatatatattgagtgaaccttgtggactctttaattagagttttcttgtatagtggcagaaataacctatttctgcattaaaatgtaatgacgtacttatgctaaactttttataaaagtttagttgtaaacttaacccttttatacaaaataaatcaagtgtgtttattgaatgttgattgcttgctttatttcagacaaccagtgctttgatttttttttatgctatgttataactgaacccaaataaataccagttcaaatttatgtagactgtattaagattataataaaatgtgtctgacatcaaaaaaaaaaaaaaaaaaaaaaaaaaaaaaaa

SEQ ID NO: 68 - protein, 134 aa, Mus musculus
MKAFSPVRSVRKNSLSDHSLGISRSKTPVDDPMSLLYNMNDCYSKLKELVPSIPQNKKVTKMEILQHVIDYILDLQIALDSHPTIVSLHHQRPGQNQASRTPLTTLNTDISILSLQASEFPSELMSNDSKVLCG

SEQ ID NO: 69 - DNA, 4654 bp, Mus musculus
ttgttcttagggtaagagagtaggaaacttgctccccatctgataagacagagtgcaaaggagaccctatttcttaggggcacagctgatctccagatatgaccatgggtttgtggctcaaacttctggcctttggatttgcccttctggacacagaagtctttgtcacagggcaaacacctacacccagtgatggtgccagcctcacaactcttacaccatccactctgggccttgcaagcactgaccctccaagcacaaccatagctaccacaacgaagcaaacatgtgctgccatgtttgggaacattactgtgaattacacctatgaatctagtaatcagacttttaaggcagacctcaaagatgtccaaaatgctaagtgtggaaatgaggattgtgaaaacgtgttaaataatctagaagaatgctcacagataaaaaacatcagtgtgtctaatgactcatgtgctccagctacaactatagatttatatgtaccaccagggactgacaagttttcgctacatgactgcacaccaaaagaaaaggctaatacttcaatttgtttggagtggaaaacaaaaaaccttgatttcagaaaatgcaacagtgacaatatttcatatgtactccactgtgagccagaaaataatacaaaatgcattagaagaaatacattcatacctgaaagatgtcagttggacaaccttcgtgcccaaacaaattacacatgtgtagcagaaatcttatatcgcggtgtaaaactcgtcaaaaatgttataaatgtgcagacagatttggggattccagaaacgcctaagcctagttgtggggatccagctgcaagaaaaacgttagtctcttggcctgagcctgtatctaaacctgagtctgcatctaaaccccatggatatgttttatgctataagaacaattcagaaaaatgtaaaagtttgcctaataatgtgaccagttttgaggtggaaagcttgaaaccttataaatactatgaagtgtccctacttgcctatgtcaatgggaagattcaaagaaatgggactgctgagaagtgcaattttcacacaaaagcagatcgtccggacaaggtcaatggaatgaaaacctcccggccgacagacaatagtataaatgttacatgtggtcctccttatgaaactaatggccctaaaaccttttacattttggtagtcagaagtggaggttcttttgttacaaaatacaacaagacaaactgtcagttttatgtagataatctctactattcaactgactatgagtttctggtctcttttcacaatggagtgtacgagggagattcagttataagaaatgagtcaacaaattttaatgctaaagcactgattatattcctggtgtttctgattattgtgacatcaatagccttgcttgttgttttgtataaaatctatgatctgcgcaagaaaagatccagcaatttagatgaacaacaggaactcgttgaaagggatgatgaaaagcagctgatggatgtggagccaatccattctgacattttgttggaaacatacaaaaggaagattgctgatgagggcagactgttcctggctgaatttcagagcattccacgggtattcagcaagtttcccatcaaagatgcccgaaagccccacaatcagaataaaaaccgttatgttgacattcttccctatgattataaccgtgtggaactctctgaaataaatggagatgcagggtccacctacataaatgccagctacattgatggcttcaaggaacccaggaaatacattgctgcacaagggccccgggatgagacagttgatgacttctggaggatgatctgggagcaaaaggccacagttattgtcatggtcacacgatgtgaagaaggaaacaggaacaagtgcgcagaatactggccaagcatggaggaaggcactcgggctttcaaagatattgttgtgacaatcaatgaccacaaacgatgtcctgattacatcattcagaagctgaacgttgcacataaaaaagaaaaagcaactggaagagaagtgactcatatccaattcaccagctggccagaccatggggttcctgaagaccctcacctgctcctcaaacttcgacggagagttaatgcttttagcaacttcttcagtggtcccattgtggtgcactgcagtgctggtgttgggcgtacaggtacctacattggaattgatgccatgctggaaggcctggaagcagagggcaaagtggatgtctatggttatgttgtcaagctaaggcgacagaggtgtctgatggtgcaagtggaggcacagtatatcctgattcatcaggctttagtggaatacaatcagtttggagaaacagaagtgaacttgtctgagttacattcatgcctacacaacatgaagaagagagatccacccagtgacccctcccctctggaggctgaataccagagacttccttcatacaggagttggaggacacagcacattggaaatcaagaagaaaataagaagaagaacaggaattctaatgttgttccatatgactttaacagagtgccacttaagcatgaactggagatgagcaaagagagtgagcctgaatcagatgagtcttcagatgatgacagtgactcagaagaaaccagcaaatacattaatgcatcctttgtgatgagttactggaaaccagaaatgatgattgctgctcaggggccactaaaagaaacgatcggtgacttttggcagatgatattccaaagaaaagtcaaagttattgtgatgttgacagagttagtgaatggagaccaggaagtctgtgctcagtactggggcgaaggaaagcagacttatggagacatggaagtggagatgaaagacacaaacagagcctcagcctacactctccgaacttttgagctgagacattccaagaggaaggagcccagaactgtgtaccagtaccagtgtaccacatggaaaggggaagagctgcctgcagaacccaaagacctggtgtctatgattcaggacctcaaacagaagcttcccaaggcttccccagaagggatgaagtatcacaagcatgcatccatcctcgtccactgcagagatggatcccagcagacagggttgttctgtgccttgttcaatctcttggaaagtgcagaaacagaagatgtggttgatgttttccaagtggtaaagtctctacgcaaagcacggcctggggtggtgtgcagctatgagcaataccagttcctctatgacatcatcgccagcatctatcccgcccagaatggacaagtcaagaaaacaaacagccaagacaaaattgaatttcataatgaagtggatggaggcaagcaggatgctaactgtgtccgtccagatggtcctctgaataaagcccaggaagacagcagaggggtgggaaccccggagcctaccaatagtgctgaggaaccagaacatgctgccaatggttctgcgagcccagctccaacccagagttcataggaaaggagtcatgtgggacaacgcagactctcacattagttctttctatttttctagacctaatgaaagaacatggctgtgcagtggtttatggaatctgtgttcacctttgccactgtataaaaatatttaagtttgtcaaaacattttgtacagttttatgcttattttaaaagtgtatctatgtcattcagcaggaatgtatatgtgagagagggtgtctgtgtgtgtgagagtgtgtttatgtatgagtgactgtgtgtgtgcatgtttgtgcgtgtgtatgacatctaaatgtgattggagaatactttcaagccatttcaaatgctttcgagaaacagtgtgccttttctcctcttgaggaaactatacattttatatctaaactgttaatttgtttgagggattaattttttaaaatcccattgaaagtggattcagttgtaagaataacaatgtgtaccattctggaatgacctcaaggtgtcctccttgtcctgttgatgatcttgtagtttaagatgctctttttggatatagataagcgtatgtaagagtgctgtgggtgtgtacagctgatctgggacgtgaacaaaatcaacatgtgagacttatgttccatatactgtcatttcatcactatctcttaatgcatatttaatcaaacatgaaaatctcaagggagactatttttgtatccacatgggaagtagaacattgcaagtcagttgctgtctacacaatagataaaaattactagttaatgctcttggtcatatcgatatatgctatgaacctaaataattgcccttagccaaatataatgtatgttaaaaacacatagaataaaaacaggggcatgaaaacttgtttgtactgaatatttacataggtaacctcgtacagttagttctgttatggaattcaccatttatgggaaatgtaaaattgactatggccatttcctatgcttaagaccatctttgacttgcattactgtgtatttatcttgaatttccccactgttttgtttactcttactgagatataatattgataaccataataaactttcaactattatcttc

SEQ ID NO: 70 - protein, 1152 aa, Mus musculus
MGLWLKLLAFGFALLDTEVFVTGQTPTPSDGASLTTLTPSTLGLASTDPPSTTIATTTKQTCAAMFGNITVNYTYESSNQTFKADLKDVQNAKCGNEDCENVLNNLEECSQIKNISVSNDSCAPATTIDLYVPPGTDKFSLHDCTPKEKANTSICLEWKTKNLDFRKCNSDNISYVLHCEPENNTKCIRRNTFIPERCQLDNLRAQTNYTCVAEILYRGVKLVKNVINVQTDLGIPETPKPSCGDPAARKTLVSWPEPVSKPESASKPHGYVLCYKNNSEKCKSLPNNVTSFEVESLKPYKYYEVSLLAYVNGKIQRNGTAEKCNFHTKADRPDKVNGMKTSRPTDNSINVTCGPPYETNGPKTFYILVVRSGGSFVTKYNKTNCQFYVDNLYYSTDYEFLVSFHNGVYEGDSVIRNESTNFNAKALIIFLVFLIIVTSIALLVVLYKIYDLRKKRSSNLDEQQELVERDDEKQLMDVEPIHSDILLETYKRKIADEGRLFLAEFQSIPRVFSKFPIKDARKPHNQNKNRYVDILPYDYNRVELSEINGDAGSTYINASYIDGFKEPRKYIAAQGPRDETVDDFWRMIWEQKATVIVMVTRCEEGNRNKCAEYWPSMEEGTRAFKDIVVTINDHKRCPDYIIQKLNVAHKKEKATGREVTHIQFTSWPDHGVPEDPHLLLKLRRRVNAFSNFFSGPIVVHCSAGVGRTGTYIGIDAMLEGLEAEGKVDVYGYVVKLRRQRCLMVQVEAQYILIHQALVEYNQFGETEVNLSELHSCLHNMKKRDPPSDPSPLEAEYQRLPSYRSWRTQHIGNQEENKKKNRNSNVVPYDFNRVPLKHELEMSKESEPESDESSDDDSDSEETSKYINASFVMSYWKPEMMIAAQGPLKETIGDFWQMIFQRKVKVIVMLTELVNGDQEVCAQYWGEGKQTYGDMEVEMKDTNRASAYTLRTFELRHSKRKEPRTVYQYQCTTWKGEELPAEPKDLVSMIQDLKQKLPKASPEGMKYHKHASILVHCRDGSQQTGLFCALFNLLESAETEDVVDVFQVVKSLRKARPGVVCSYEQYQFLYDIIASIYPAQNGQVKKTNSQDKIEFHNEVDGGKQDANCVRPDGPLNKAQEDSRGVGTPEPTNSAEEPEHAANGSASPAPTQSS

SEQ ID NO: 71 - DNA, 813 bp, Mus musculus
tcagatcagacagaggactcctggacaaagccgctgcttgctagctgcttctctggctcagaagacgaggaattcgggtgcatggcctttttctttttctaagagccaacagtctgggtcatctgtgtctttgtcccctaagaagtggaaacccagagctgtgtccatggagccctgccggtccctggccctgtttgctggctctctgggcctgacttcttctctgattgctctgaccactgatttctggatagtggccacaggtcctcacttctctgcccactctggcctctggccaacaagccaagagactcaagtagcaggttatatccatgtgacacagagcttctgtatcctggctgtcctgtggggcctggtgtctgtgagcttcctggttctgtcttgcatcccagccctgtctgctcccggccgtggccctctggtctcaactgtcatggctttttctgcagctctctccatattagtggctatggcagtgtacaccagtatgagatggagccagactccattttctcaagtccagacattcttctcctggtccttctacctaggctgggtctccttcatcctcttcctctttgcaggctgcctgagcctgggtgctcactgtagaacccgtcgggctgagtatgaaaccttgtgaacagcagccaaggacagcagggtcatcaggatgtttggagcctgtcctctcaggagctacaaaaggaatgcagaattctgtctctgtccctcccaccttgccttatccttcattggtttcttcacacactcaataaagaagcatgcttg

SEQ ID NO: 72 - protein, 165 aa, Mus musculus
MEPCRSLALFAGSLGLTSSLIALTTDFWIVATGPHFSAHSGLWPTSQETQVAGYIHVTQSFCILAVLWGLVSVSFLVLSCIPALSAPGRGPLVSTVMAFSAALSILVAMAVYTSMRWSQTPFSQVQTFFSWSFYLGWVSFILFLFAGCLSLGAHCRTRRAEYETL

SEQ ID NO: 73 - DNA, 1239 bp, Mus musculus
agacacacagcagcagcagcagcagcagcagcagcaacagcagcagcagcagcagcgcctgtgggaaaaactagaggctagagccatggatgaccaacgcgacctcatctctaaccatgaacagttgcccatactgggcaaccgccctagagagccagaaaggtgcagccgtggagctctgtacaccggtgtctctgtcctggtggctctgctcttggctgggcaggccaccactgcttacttcctgtaccagcaacagggccgcctagacaagctgaccatcacctcccagaacctgcaactggagagccttcgcatgaagcttccgaaatctgccaaacctgtgagccagatgcggatggctactcccttgctgatgcgtccaatgtccatggataacatgctccttgggcctgtgaagaacgttaccaagtacggcaacatgacccaggaccatgtgatgcatctgctcacgaggtctggacccctggagtacccgcagctgaaggggaccttcccagagaatctgaagcatcttaagaactccatggatggcgtgaactggaagatcttcgagagctggatgaagcagtggctcttgtttgagatgagcaagaactccctggaggagaagaagcccaccgaggctccacctaaagagccactggacatggaagacctatcttctggcctgggagtgaccaggcaggaactgggtcaagtcaccctgtgaagacagaggccagctctgcacagcagcagcgccccctgctctcctgtgcctcagcccttcttatgttccctgatgtcacaccccacttcccgtctccctgcaccctggggcttgagactggtgtctgtttcatcgtcccaggacacggcaaatgaagtcagaacagaaggaggacgctggagggccttgctggctaccgctatctaaagggaacccccatttctgacccattagtagtcttgaatgtggggctctgagataaaggcccgcagacagggacaagggatgccctacccttaacctaggctggacacatttgctgccttctcctcaaggaagaagaacccaagcccctcctcccagtaacccctcctcacatcctgccaccccccctcaagccccaccccctttcaggttccttgctcagccaagcttgtcagcagcctgtaggatcatggttcaagtgacaataaaggaagaaagtagaacaaaaaaaaaaaaaaaaa

SEQ ID NO: 74 - protein, 215 aa, Mus musculus
MDDQRDLISNHEQLPILGNRPREPERCSRGALYTGVSVLVALLLAGQATTAYFLYQQQGRLDKLTITSQNLQLESLRMKLPKSAKPVSQMRMATPLLMRPMSMDNMLLGPVKNVTKYGNMTQDHVMHLLTRSGPLEYPQLKGTFPENLKHLKNSMDGVNWKIFESWMKQWLLFEMSKNSLEEKKPTEAPPKEPLDMEDLSSGLGVTRQELGQVTL

SEQ ID NO: 75 - DNA, 742 bp, Mus musculus
tgacttgcgaagtgggctgcagagttggaccgggctgctgcgtgtgcgcagggtcctcgtcatggggcagatcgagtgggccatgtgggccaacgaacaggcgctggcgtctggcctgattctcatcactgggggcatcgtggctactgctggacgtttcacacagtggtatttcggcgcctactctatcgctgcaggtgtgctcatctgtctgctggagtatccccggggaaagaggaaaaaggggtccaccatggagcgatgtggacagaagtacctgacccctgtggtgaagcttttcgggcccctcaccaggaattactacgtccgggctgccctccacttcctgttgtcggtgcctgcaggcttcctcctggccaccatcctggggaccgtctgcttggccattgccagtgtgatctatctgctggcagccatccgaggtgagcagtggactcccattgagcctaaacccaaggagcggccacaggttggaggcaccatcaagcaaccacctaccaaccccccaccccggccacccgcagaggtccgaaagaagccgagtgagggtgaagaggaggcagcctcagctggaggaccccaggttaacccaatgccagtgacagatgaggtcgtgtgaccttcagtggctgtctacagctcctgcttgagtttctgtggagttgtccccccccccccagggtggtgttgctcactgtaataaacatgattaatagctggt

SEQ ID NO: 76 - protein, 192 aa, Mus musculus
MGQIEWAMWANEQALASGLILITGGIVATAGRFTQWYFGAYSIAAGVLICLLEYPRGKRKKGSTMERCGQKYLTPVVKLFGPLTRNYYVRAALHFLLSVPAGFLLATILGTVCLAIASVIYLLAAIRGEQWTPIEPKPKERPQVGGTIKQPPTNPPPRPPAEVRKKPSEGEEEAASAGGPQVNPMPVTDEVV

SEQ ID NO: 77 - DNA, 2953 bp, Mus musculus
ggaggagaatgagattcatggagaagaacacgacaggccagggctgctaggcagaactccaactacagcttcagcggcagcttccagaacagcctgagcaagccagtctcagaaggaggcgtgtctagtgattcgaggtcggctttcggtttcttcttcctctaaacgccagcacttctagtcagctccaccagctcgagcgggttcccgggactttacgcgcacgccctcggacccgcccttcttccttccccacggagactcctgtgcagcgcggacgtcgagagtcccaggctaggaccagactctggacagctcacgctcgatggctgcgcacgtctggctggcggccgccctgctccttctggtggactggctgctgctgcggcccatgctcccgggaatcttctccctgttggttcccgaggtgccgctgctccgggtctgggtggtgggcctgagtcgctgggccatcctaggactaggggtccgcggggtcctcggggtcaccgcaggagcccatggctggctggctgctttgcagccgctggtggccgcactgagtttggccctgcctggacttgccttgttccgagagctggccgcctggggaacactccgggagggtgacagcgctggattactgtactggaacagtcgtccagatgccttcgctatcagttatgtggcagcattgcccgcagccgccctgtggcacaagttggggagcctctgggcgcccagcggcaacagggacgctggagacatgctgtgtcggatgctgggcttcctgggccctaagaagagacgtctctacctggttctggttctcttgattctctcttgccttggggaaatggccattcccttcttcacgggccgcatcactgactggattcttcaggataagacagttcctagcttcacccgcaacatatggctcatgtccattctcaccatagccagcacagcgctggagtttgcaagtgatggaatctacaacatcaccatgggacacatgcacggccgtgtgcacagagaggtgtttcgggccgtccttcgccaggagacagggtttttcctgaagaacccagcaggttccatcacatctcgggtgactgaggacacagccaacgtgtgcgagtccattagtggcacgctgagcctgctgctgtggtacctggggcgagccctgtgtctcttggtgttcatgttttgggggtcaccgtacctcactctggtcaccctgatcaacctgcccctgctttttcttttgcctaagaagctgggaaaagtgcatcagtcactggcagtgaaggtgcaggagtctctagcaaagtccacgcaggtggcccttgaggccttatcggcgatgcctactgtgcggagctttgccaacgaggagggtgaggcccagaagttcaggcagaagttggaagaaatgaagactctaaacaagaaggaggccttggcttacgtggctgaagtctggaccacgagtgtctcgggaatgctgctgaaggtgggaattctgtacctgggcgggcagctggtgatcagaggggctgtcagcagcggcaaccttgtctcattcgttctctaccagcttcagttcacccaggctgttcaggtcctgctctccctctacccctccatgcagaaggctgtgggctcctcagagaaaatattcgaatacttggaccggactccttgctctccactcagtggctcgttggcaccctcaaacatgaaaggccttgtggagttccaagatgtctcttttgcctacccaaaccagcccaaagtccaggtgcttcaggggctgacgttcaccctgcatcctggaacggtgacagcgttggtgggacccaatggatcagggaagagcaccgtggctgccctgctgcagaacctgtaccagcccaccgggggccagctgctgctggatggccagtgcctggtccagtatgatcaccattacctgcacactcaggtggccgcagtgggacaagagccgctgctatttggaagaagctttcgagaaaatattgcgtatggcctgaaccggactccaaccatggaggaaatcacagctgtggccgtggagtctgggagcccacgatttcatctctgggttccctcagggctatgacacagaggtaggtgagactgggaaccagctgtcaggaggtcagcgacagggcagtggccttggcccgagccttgatccggaagccactcctgcttatcttggatgatgccaccagtgccctggatgctggcaaccagctacgggtccagcggctcctgtatgagagccccaagcgggcttctcggacggttcttcttatcacccagcagctcagcctggcagagcaggcccaccacatcctctttctcagagaaggctctgtcggcgagcagggcacccacctgcagctcatgaagagaggagggtgctaccgggccatggtagaggctcttgcggctcctgcagactgacaaggcctctggactgcacactgcgtgctgtccccctcctgtcctctactctgtgtgacagagaacctgggagcaaagatattaccacacccacggagacagttgaggagcggaggtgcttgttacatgagaaaatgtaaactctaggagatacccggaatttaccacgaatgtgtttccccgacccgctccctattagacggggtttcaggtacctcacaccaacactgagctgctaaatctccggtctccaccctcccggtgctgagcttgtatcacagcatgcacacacaaacctggtttatgtggcgttgtgatagaatgagaagaaacgcccaaagtgtacagagagaaaggacaagcagcttgcaattaaccaaaggcataggctggcatttgggtgttctatgggtgtttgatatttgtaataaaactggtgttttgtactgtg

SEQ ID NO: 78 - DNA, 627 bp, Mus musculus
cggccgccagccagacgtgcgcagccatcgagcgtgagctgtccagagtctggtcctagcctgggactctcgacgtccgcgctgcacaggagtctccgtggggaaggaagaagggcgggtccgagggcgtgcgcgtaaagtcccgggaacccgctcgagctggtggagctgactagaagtgctggcgtttagaggaagaagaaaccgaaagccgacctcgaatcactagacacgcctccttctgagactggcttgctcaggctgttctggaagctgccgctgaagctgtagttggagttctgcctagcagccctggcctgtcgtgttcttctccatgaatctcattctcctcttcttccccagcccccttaacttgcttgcgtgaattttcccatcccaggccccaccccagattgagggcaggaccctctgtcgtcacctgtctctgggcagatttgccctgctcaggtttgctctgggcgccaaatgctggtagaacggcacgccccttgcgcgatgatggggcggggcctcagtctgcaagttgcctgcaccgcgcgcagccgctgattgggaagccgagccccgctctgctgaaatgctgcgggcaggagcacctaccgccgg

SEQ ID NO: 79 - DNA, 2772 bp, Mus musculus
agtcagctccaccagctcgagcgggttcccgggactttacgcgcacgccctcggacccgcccttcttccttccccacggagactcctgtgcagcgcggacgtcgagagtcccaggctaggaccagactctggacagctcacgctcgatggctgcgcacgtctggctggcggccgccctgctccttctggtggactggctgctgctgcggcccatgctcccgggaatcttctccctgttggttcccgaggtgccgctgctccgggtctgggtggtgggcctgagtcgctgggccatcctaggactaggggtccgcggggtcctcggggtcaccgcaggagcccatggctggctggctgctttgcagccgctggtggccgcactgagtttggccctgcctggacttgccttgttccgagagctggccgcctggggaacactccgggagggtgacagcgctggattactgtactggaacagtcgtccagatgccttcgctatcagttatgtggcagcattgcccgcagccgccctgtggcacaagttggggagcctctgggcgcccagcggcaacagggacgctggagacatgctgtgtcggatgctgggcttcctgggccctaagaagagacgtctctacctggttctggttctcttgattctctcttgccttggggaaatggccattcccttcttcacgggccgcatcactgactggattcttcaggataagacagttcctagcttcacccgcaacatatggctcatgtccattctcaccatagccagcacagcgctggagtttgcaagtgatggaatctacaacatcaccatgggacacatgcacggccgtgtgcacagagaggtgtttcgggccgtccttcgccaggagacagggtttttcctgaagaacccagcaggttccatcacatctcgggtgactgaggacacagccaacgtgtgcgagtccattagtggcacgctgagcctgctgctgtggtacctggggcgagccctgtgtctcttggtgttcatgttttgggggtcaccgtacctcactctggtcaccctgatcaacctgcccctgctttttcttttgcctaagaagctgggaaaagtgcatcagtcactggcagtgaaggtgcaggagtctctagcaaagtccacgcaggtggcccttgaggccttatcggcgatgcctactgtgcggagctttgccaacgaggagggtgaggcccagaagttcaggcagaagttggaagaaatgaagactctaaacaagaaggaggccttggcttacgtggctgaagtctggaccacgagtgtctcgggaatgctgctgaaggtgggaattctgtacctgggcgggcagctggtgatcagaggggctgtcagcagcggcaaccttgtctcattcgttctctaccagcttcagttcacccaggctgttcaggtcctgctctccctctacccctccatgcagaaggctgtgggctcctcagagaaaatattcgaatacttggaccggactccttgctctccactcagtggctcgttggcaccctcaaacatgaaaggccttgtggagttccaagatgtctcttttgcctacccaaaccagcccaaagtccaggtgcttcaggggctgacgttcaccctgcatcctggaacggtgacagcgttggtgggacccaatggatcagggaagagcaccgtggctgccctgctgcagaacctgtaccagcccaccgggggccagctgctgctggatggccagtgcctggtccagtatgatcaccattacctgcacactcaggtggccgcagtgggacaagagccgctgctatttggaagaagctttcgagaaaatattgcgtatggcctgaaccggactccaaccatggaggaaatcacagctgtggccgtggagtctggagcccacgatttcatctctgggttccctcagggctatgacacagaggtaggtgagactgggaaccagctgtcaggaggtcagcgacaggcagtggccttggcccgagccttgatccggaagccactcctgcttatcttggatgatgccaccagtgccctggatgctggcaaccagctacgggtccagcggctcctgtatgagagccccaagcgggcttctcggacggttcttcttatcacccagcagctcagcctggcagagcaggcccaccacatcctctttctcagagaaggctctgtcggcgagcagggcacccacctgcagctcatgaagagaggagggtgctaccgggccatggtagaggctcttgcggctcctgcagactgacaaggcctctggactgcacactgcgtgctgtccccctcctgtcctctactctgtgtgacagagaacctgggagcaaagatattaccacacccacggagacagttgaggagcggaggtgcttgttacatgagaaaatgtaaactctaggagatacccggaatttaccacgaatgtgtttccccgacccgctccctattagacggggtttcaggtacctcacaccaacactgagctgctaaatctccggtctccaccctcccggtgctgagcttgtatcacagcatgcacacacaaacctggtttatgtggcgttgtgatagaatgagaagaaacgcccaaagtgtacagagagaaaggacaagcagcttgcaattaaccaaaggcataggctggcatttgggtgttctatgggtgtttgatatttgtaataaaactggtgttttgtattgtg

SEQ ID NO: 80 - DNA, 2431 bp, Mus musculus
atggtcgtaattcacctacatgacatggcggcgccgttgcccaagaatgtgatgcttctctttcatcttcaacaggcttggaagtgtgagccgcccattgcaggtgaacagagcctggtctctatggagtccatgtggggctttgcagaccagcgcctctgctttaaggagaagcctatcttcactgcatccccaagtgcttgtgtctccattgtattcccagaagtggcatttcttctagaagactccaaggtgatcaccgggaaccaatcagcgtcggcgcgggagcgggttgtaaagtccacgctgcccacaggagtcagtcgacctgaatccccgagaggagcaatggctctaacaacgctgctcttgctggtggcggccgccctgaccctgatcgagacccgcgcgggcccacactcgctgcggtatttccacactgctgtgtcctggcccggcctcgtggagccccggttcattatcgtcggctacgtggacaacaagcagttcgtgcgcttcgacagcgacgcggaaaatccgaggatggagccgcgggcgcggtggatggagcaggaggggccggagtattgggagcgggagacacagaaagccaagggccatgaggagagtttccgagtgagcctgaggaccgcacagagatactacaaccagagcaagggcggctctcacacactccagtggatgtatggctgtgacgtggggtccgacgagcgcctcctccgcgggtacctgcagttcgcctatgaaggccgcgattacatcgccctgaacgaagacctgaaaacctggacggcggcggacatggctgcacagatcaccctacacaagtgggagcaggctggtattgcagagagagaccgggcctacctggagggcgcttgcgtgcagtcgctccgcagatacctgcagctccggaaggagacgctgctgtgcacagatcccccaaaggcacatgtgacccatcaccccaggtcttatggtgctgtcaccctgaggtgctgggccctgggcttctaccctgctgacatcaccctgacttggcagttgaatggggaggagctgacccaggacatggagcttgtggagaccaggcctgcaggggatggaaccttccagaagtgggcatctgtggtggtgcctcttgggaaggagcagaattacacatgccatgtgaaccatgaggggctgcctgagcctctcaccctgagatgggagcctcctccatccactgtctccaacatggcgaacgtagctattctggttgtccttgtagcttggccatcattggagctgtggtggattttgtgatgaagagaaggagacacacaggtggacaaggaggggactatgctctggctccagagaacagtgtctgatgttccctgtgagcctatgggctcaatgtgaagaattgtggagcccagccttcgcctacacaccaggaccctgtctctgcattgccctgtgttcccttccaccgccaaccttcctggtctgcagtggaaactaagggttctttggaaagtcggttggatggcgttgtgctggggcaaacacatgaaggagtgtttccttaaagtggatacaggtgtgaaatcctaaggccgactcctgaaggaacatttcactgaagcagacacaggagagaggatgttctgttaaacaagcatgtgaaaggatgcgtgatgaaggattctttgctaaagacacacttatattggtctgccttacattgtgtagtggagctgcattggtcggggcgccatagagagaaacgcacccaaaaattctgatagtattctgcagttgatgcttccaaggacatgagcttattggatgcctgtgctgaggcaagacgcttggaggacacatgatgactggagagtataaataggactccatggagtgactgagacagagtttgccttgctggtacagtgtgtgtcttgtgtcttcactgttcttcacttccataagagaggtacagcaaagaacttctcctggtattcctgctggtccttcctgctggccctagctgaggcctggctgtctctgctaggtcctatcacttctgttgctaacctgactctactgaactgaacaactgctatatccctgaggtgttttcaagtggatggagctgccactgctgacctgtgaattgaactgccaacgtccagacaactcacatgagagttgctcccaagtgtagtctcccctcccccagcctgaaacctgcttgctcaggggtggagcttcctgctcattcgttctgccacgcccactgctagaacctgcggagccacacacgtgcacctttctactggaccagagattattcggcgggaatcgggtcccctcccccttccttcataactagtgtcgcaacaataaaatttgagctttgatc

SEQ ID NO: 81 - protein, 465 aa, Mus musculus
  misc_feature (443)..(444): Xaa can be any naturally occurring amino acid
MVVIHLHDMAAPLPKNVMLLFHLQQAWKCEPPIAGEQSLVSMESMWGFADQRLCFKEKPIFTASPSACVSIVFPEVAFLLEDSKVITGNQSASARERVVKSTLPTGVSRPESPRGAMALTTLLLLVAAALTLIETRAGPHSLRYFHTAVSWPGLVEPRFIIVGYVDNKQFVRFDSDAENPRMEPRARWMEQEGPEYWERETQKAKGHEESFRVSLRTAQRYYNQSKGGSHTLQWMYGCDVGSDERLLRGYLQFAYEGRDYIALNEDLKTWTAADMAAQITLHKWEQAGIAERDRAYLEGACVQSLRRYLQLRKETLLCTDPPKAHVTHHPRSYGAVTLRCWALGFYPADITLTWQLNGEELTQDMELVETRPAGDGTFQKWASVVVPLGKEQNYTCHVNHEGLPEPLTLRWEPPPSTVSNMANVAILVVLVAWPSLELWWILXXREGDTQVDKEGTMLWLQRTVS

SEQ ID NO: 82 - DNA, 3390 bp, Mus musculus
gctcctctcccaccgagccgggcctgggcgcctcccggccaacgttctgcagctaataggttgctgcaggccgggcactgttccccgagcagtgtgcccgcaagtcccggtcacgcgtcctaagacaccggcccgcagctgcttcccaggcgggccccgcagggctatcccgtagcgtgaacagcttttaaccgaagtcaccgtggagttccgccgccccgaaacttccaccacgagcgagaaatatgggatgtattaaatcaaaaaggaaagacaatctcaatgacgatgaagtagattcgaagactcaaccagttcctgaatttcatcttttaccaggacagagatttcaaacaaaagatccagaggaacaaggtgacattgtggtggccttatacccttatgatggcatccacccagatgacttgtccttcaagaaaggagaaaagatgaaagttctagaagagcatggggaatggtggaaagctaagtccctttcatcaaagagagaaggcttcatccccagcaactacgtggccaaggtcaacaccttagaaactgaagagtggttcttcaaggacataacaaggaaagattcagagcgacagcttctggcaccagggaacagtgcaggagctttccttatcagagaaagcgaaactttaaagggaagcttctctctttctgtcagagattatgaccctatgcatggtgatgtcattaagcactacaaaattagaagtctggacaatggtggctattacatctctcctcgcatcacttttccctgcatcagtgacatgattaagcattaccaaaagcagtctgatggtctatgcagaagactggagaaggcatgcatcagtcccaaacctcagaagccatgggataaagatgcctgggagatcccccgggagtccattaagttggtgaaaaagcttggcgcagggcagtttggggaagtctggatgggttactataacaacagcacaaaggtggctgtgaagaccctcaagcccggcaccatgtctgtgcaggcattcctggaagaggccaacctcatgaagaccttgcaacatgacaagctagtgcggctgtacgctgtggtcaccaaggaggagcccatctacatcatcaccgagttcatggctaagggtagtttgctggatttcctcaagagtgatgaaggtggcaaggtgctgctgcccaagctcattgacttctcggcccagattgcagaaggcatggcgtacatcgagcggaagaactacatccaccgtgatctgcgagctgctaacgtcctggtctctgagtcactcatgtgcaagattgcagactttggcctcgcgagagtcatcgaagataacgagtacacagcaagggaaggtgcgaagttccctatcaagtggacagctccagaggccatcaacttcggctgcttcactatcaaatctgacgtgtggtccttcggaattctcctgtatgagattgtcacctatgggaagattccctacccagggagaaccaacgcagatgtgatgagcgcactgtcacagggatatcgaatgccacgcatggagaactgcccagatgagctctatgacatcatgaaaatgtgttggaaagaaaaggcacaggagaggccaacttttgactacttacagagtgtcctggatgacttctatacagccacagaagggcactatcagcagcaaccgtagtgggcattgagactagcctattgtaggtagttgggaaagcagccttggaggagctaactatctcatgccaggaccctctgtgttcctggttcctgatggacgtgatgaattaactcaggaacaacaccctggatgctgtttcttaaatgggtggccgctgctcaatgggaccacagtcagaatacaaacaccctctctttctaatattcacactggctctacaggggccgagtgactcaggttcagagactgttgcaacaaattcccagatgcaacaagaactaaactcacttagcaaaaataaaccccatgctgagcagatgctcccgtaccttctcaatgctttggcttacattcttaaaaactactaatccagcctgttagattttacaggtgaagccacaagctttaaaacaccttttctaaatttgaatttttttttttttttgctttggaaatttgattcctcctcaccttccgcacaacccccagaaaatgagactattgaacattttcctttgtggtaagatcactggacaggaggcagcacgtgctcactctgctggagcgctgtgcatgggcacacagagggaccactgctgaactggagccctttggagctacaccactgcccagacctccctcgaaggcaggaaagcctcaagctctaaaggaatgtttttaatggtccaggtatttcaattatgggattctctaaaggctttcctgatggtgtcctttgggagaaatgtaaacatccatctaatctgtcaggactttatgagaccttccaagaatacaaagcatttctctcattgcctcaaatgacttgaacattttataaatcatatattttagttatactctaaaaaagttaaaatggcatatttcgggttattcttgtgactcggtaatcttttctaaaatgtgtaaagtgtatgagagactatttacatgtaggaatcaggagtcatggaaaagcctctggtgaccagagcagttttgttacctgagttgcccactgtcaacgctgattggaaactgctgtgtctgcccctttcacttatgctactgtgtacatacttaatattttgatttttgattttattttaatacacccaataacatcttaagatttttgtcttgtgtatcagattctcaactcttgtccatgtataaataagagctggttggtgggggaagggcagggagtcattctgtacagtctactgcatgaacttgtgcaggctgcacctccacaactggagcatttcaggttggagggctgaggagttgatggcctgggtagccacagatctgtttttaacaggctttgcaggcaccattaaaattgaaatttgtatgttcatttgaagatctagatgactaggcgaagggattgaatatctcatttttttctcaatctttgtcttttccatgattttttaaaatagtatcatttcaaaatattgctaaagccctttatatccgagtgcctatttccaaacgcagcatgtgtacatttattcatttgattgacaatagtgggatttctaaggccctccagtccctactctcatttcaaactcacctgcattgtctactttatttcatggaaaggaagtgcgaagaatccttaaagaaatcactaaaacattgctaaaaattc

SEQ ID NO: 83 - protein, 491 aa, Mus musculus
MGCIKSKRKDNLNDDEVDSKTQPVPEFHLLPGQRFQTKDPEEQGDIVVALYPYDGIHPDDLSFKKGEKMKVLEEHGEWWKAKSLSSKREGFIPSNYVAKVNTLETEEWFFKDITRKDSERQLLAPGNSAGAFLIRESETLKGSFSLSVRDYDPMHGDVIKHYKIRSLDNGGYYISPRITFPCISDMIKHYQKQSDGLCRRLEKACISPKPQKPWDKDAWEIPRESIKLVKKLGAGQFGEVWMGYYNNSTKVAVKTLKPGTMSVQAFLEEANLMKTLQHDKLVRLYAVVTKEEPIYIITEFMAKGSLLDFLKSDEGGKVLLPKLIDFSAQIAEGMAYIERKNYIHRDLRAANVLVSESLMCKIADFGLARVIEDNEYTAREGAKFPIKWTAPEAINFGCFTIKSDVWSFGILLYEIVTYGKIPYPGRTNADVMSALSQGYRMPRMENCPDELYDIMKMCWKEKAQERPTFDYLQSVLDDFYTATEGHYQQQP

SEQ ID NO: 84 - DNA, 2086 bp, Mus musculus
atgagccagtgtgctcagccaaaggtctctcctggacctgtggcagttttgctctcttcgattgcagaaggcatggcgtacatcgagcggaagaactacatccaccgtgatctgcgagctgctaacgtcctggtctctgagtcactcatgtgcaagattgcagactttggcctcgcgagagtcatcgaagataacgagtacacagcaagggaagggagaaccaacgcagatgtgatgagcgcactgtcacagggatatcgaatgccacgcatggagaactgcccagatgagctctatgacatcatgaaaatgtgttggaaagaaaaggcagaggagaggccaacttttgactacttacagagtgtcctggatgacttctatacagccacagaagggcagtatcagcagcaaccgtagtgggcatggagactagcctattgtaggtagttggaaaagcagccttggaggagctaactatctcatgccaggaccctctgtgttcctggttcctgatggacgtgatgaattaactcaggaagaacaccctggatgctgtttcttaaatgggtggccgctgctcaatgggaccacagtcagaatacaaacaccctctctttctaatatgcacactggctctacaggggccgagtgactcaggttcagagactgttgcaacaaattcccagatgcaacaagaactaaactcacttagcaaaaataaaccccatgctgagcagatgctcccgtaccttctcaatgctttggcttacattcttaaaaactactaatccagcctgttagattttacaggtgaagccacaagctttaaaacaccttttctaaatttgaatttttttttttttttgctttggaaatttgattcctcctcaccttccgcacaacccccagaaaatgagactattgaacattttcctttgtggtaagatcactggacaggaggcagcacgtgctcactctgctggagcgctgtgcatgggcacacagagggaccactgctgaactggagccctttggagctacaccactgcccagacctccctcgaaggcaggaaagcctcaagctctaaaggaatgtttttaatggtccaggtatttcaattatgggattctctaaaggctttcctgatggtgtcctttgggagaaatgtaaacatccatctaatctgtcaggactttatgagaccttccaagaatacaaagcatttctctcattgcctcaaatgacttgaacattttataaatcatatattttagttatactctaaaaaagttaaaatggcatatttcgggttattcttgtgactcggtaatcttttctaaaatgtgtaaagtgtatgagagactatttacatgtaggaatcaggagtcatggaaaagcctctggtgaccagagcagttttgttacctgagttgcccactgtcaacgctgattggaaactgctgtgtctgcccctttcacttatgctactgtgtacatacttaatattttgatttttgattttattttaatacacccaataacatcttaagatttttgtcttgtgtatcagattctcaactcttgtccatgtataaataagagctggttggtgggggaagggcagggagtcattctgtacagtctactgcatgaacttgtgcaggctgcacctccacaactggagcatttcaggttggagggctgaggagttgatggcctgggtagccacagatctgtttttaacaggctttgcaggcaccattaaaattgaaatttgtatgttcatttgaagatctagatgactaggcgaagggattgaatatctcatttttttctcaatctttgtcttttccatgattttttaaaatagtatcatttcaaaatattctaaagccctttatatccgagtgcctatttccaaacgcagcatgtgtacatttattcatttgattgacaatagtgggatttctaaggccctccagtccctactctcatttcaaactcacctgcattgtctactttatttcatggaaaggaagtgcgaagaatccttaaagaaatcactaaaacattgctaaaaattca

SEQ ID NO: 85 - protein, 138 aa, Mus musculus
MSQCAQPKVSPGPVAVLLSSIAEGMAYIERKNYIHRDLRAANVLVSESLMCKIADFGLARVIEDNEYTAREGRTNADVMSALSQGYRMPRMENCPDELYDIMKMCWKEKAEERPTFDYLQSVLDDFYTATEGQYQQQP

SEQ ID NO: 86 - DNA, 3375 bp, Mus musculus
tcggccgagcccagcgacagccagttcctctcccaccgagccgggcctgggcgcctcccggccaacgttctgcagctaataggttgctgcaggccgggcactgttccccgagcagtgtgcccgcaagtcccggtcacgcgtcctaagacaccggcccgcagctgcttcccaggcgggccccgcagggctatcccgtcgcgtgaacagcttttaaccgaagtcaccgtggagttccgccgccccgaaacttccaccacgagcgagaaatatgggatgtattaaatcaaaaaggaaagacaatctcaatgacgatgaagtagattcgaagactcaaccagttcctgaatttcatcttttaccaggacagagatttcaaacaaaagatccagaggaacaaggtgacattgtggtggccttatacccttatgatggcatccacccagatgacttgtccttcaagaaaggagaaaagatgaaagttctagaagagcatggggaatggtggaaagctaagtccctttcatcaaagagagaaggcttcatccccagcaactacgtggccaaggtcaacaccttagaaactgaagagtggttcttcaaggacataacaaggaaagatgcagagcgacagcttctggcaccagggaacagtgcaggagctttccttatcagagaaagcgaaactttaaagggaagcttctctctttctgtcagagattatgaccctatgcatggtgatgtcattaagcactacaaaattagaagtctggacaatggtggctattacatctctcctcgcatcacttttccctgcatcagtgacatgattaagcattaccaaaagcagtctgatggtctatgcagaagactggagaaggcatgcatcagtcccaaacctcagaagccatgggataaagatgcctgggagatcccccgggagtccattaagttggtgaaaaagcttggcgcagggcagtttggggaagtctggatggatgacccctcagaaggagctggtcacatggctggggagggagcaacaatacaaatgcctgtgactctaatggaaccttcaaatgactggcttttggtggggtctgctggtgttctggagctgcagacacagctgttaagtcatggtagtttgctggatttcctcaagagtgatgaaggtggcaaggtgctgctgcccaagctcattgacttctcggcccagattgcagaaggcatggcgtacatcgagcggaagaactacatccaccgtgatctgcgagctgctaacgtcctggtctctgagtcactcatgtgcaagattgcagactttggcctcgcgagagtcatcgaagataacgagtacacagcaagggaaggtgcgaagttccctatcaagtggacagctccagaggccatcaacttcggctgcttcactatcaaatctgacgtgtggtccttcggaattctcctgtatgagattgtcacctatgggaagattccctacccagggagaaccaacgcagatgtgatgagcgcactgtcacagggatatcgaatgccacgcatggagaactgcccagatgagctctatgacatcatgaaaatgtgttggaaagaaaaggcagaggagaggccaacttttgactacttacagagtgtcctggatgacttctatacagccacagaagggcagtatcagcagcaaccgtagtgggcatggagactagcctattgtaggtagttggaaaagcagccttggaggagctaactatctcatgccaggaccctctgtgttcctggttcctgatggacgtgatgaattaactcaggaagaacaccctggatgctgtttcttaaatgggtggccgctgctcaatgggaccacagtcagaatacaaacaccctctctttctaatatgcacactggctctacaggggccgagtgactcaggttcagagactgttgcaacaaattcccagatgcaacaagaactaaactcacttagcaaaaataaaccccatgctgagcagatgctcccgtaccttctcaatgctttggcttacattcttaaaaactactaatccagcctgttagattttacaggtgaagccacaagctttaaaacaccttttctaaatttgaattttttttttttttttgctttggaaatttgattcctcctcaccttccgcacaacccccagaaaatgagactattgaacattttcctttgtggtaagatcactggacaggaggcagcacgtgctcactctgctggagcgctgtgcatgggcacacagagggaccactgctgaactggagccctttggagctacaccactgcccagacctccctcgaaggcaggaaagcctcaagctctaaaggaatgtttttaatggtccaggtatttcaattatgggattctctaaaggctttcctgatggtgtcctttgggagaaatgtaaacatccatctaatctgtcaggactttatgagaccttccaagaatacaaagcatttctctcattgcctcaaatgacttgaacattttataaatcatatattttagttatactctaaaaaagttaaaatggcatatttcgggttattcttgtgactcggtaatcttttctaaaatgtgtaaagtgtgtgagagactatttacatgtaggaatcaggagtcatggaaaagcctctggtgaccagagcagttttgttacctgagttgcccactgtcaacgctgattggaaactgctgtgtctgcccctttcacttatgctactgtgtacatacttaatattttgatttttgattttattttaatacacccaataacatcttaagatttttgtcttgtgtatcagattctcaactcttgtccatgtataaataagagctggttggtgggggaagggcagggagtcattctgtacagtctactgcatgaacttgtgcaggctgcacctccacaactggagcatttcaggttggagggctgaggagttgatggcctgggtagccacagatctgtttttaacaggctttgcaggcaccattaaaattgaaatttgtatgttcatttgaagatctagatgactaggcgaagggattgaatatctcatttttttctcaatctttgtcttttccatgattttttaaaatagtatcatttcaaaatattctaaagccctttatatccgagtgcctatttccaaacgcagcatgtgtacatttattcatttgattgacaatagtgggatttctaaggccctccagtccctactctcatttcaaactcacctgcattgtctactttatttcatggaaaggaagtgcgaagaatccttaaagaaatcactaaaacattgctaaaaattca

SEQ ID NO: 87 - protein, 478 aa, Mus musculus
MGCIKSKRKDNLNDDEVDSKTQPVPEFHLLPGQRFQTKDPEEQGDIVVALYPYDGIHPDDLSFKKGEKMKVLEEHGEWWKAKSLSSKREGFIPSNYVAKVNTLETEEWFFKDITRKDAERQLLAPGNSAGAFLIRESETLKGSFSLSVRDYDPMHGDVIKHYKIRSLDNGGYYISPRITFPCISDMIKHYQKQSDGLCRRLEKACISPKPQKPWDKDAWEIPRESIKLVKKLGAGQFGEVWMDDPSEGAGHMAGEGATIQMPVTLMEPSNDWLLVGSAGVLELQTQLLSHGSLLDFLKSDEGGKVLLPKLIDFSAQIAEGMAYIERKNYIHRDLRAANVLVSESLMCKIADFGLARVIEDNEYTAREGAKFPIKWTAPEAINFGCFTIKSDVWSFGILLYEIVTYGKIPYPGRTNADVMSALSQGYRMPRMENCPDELYDIMKMCWKEKAEERPTFDYLQSVLDDFYTATEGQYQQQP

SEQ ID NO: 88 - DNA, 1621 bp, Mus musculus
gacacagactacacccagagaaagaagagcaagcaccatgttgaaactattattgtcacctagatccttcttagtccttcagctgctcctgctgagggcagggtggagctccaaggtcctcatgtccagtgcgaatgaagacatcaaagctgatttgatcctgacttctacagcccctgaacacctcagtgctcctactctgccccttccagaggttcagtgctttgtgttcaacatagagtacatgaattgcacttggaatagcagttctgagcctcaggcaaccaacctcacgctgcactataggtacaaggtatctgataataatacattccaggagtgcagtcactatttgttctccaaagagattacttctggctgtcagatacaaaaagaagatatccagctctaccagacatttgttgtccagctccaggacccccagaaaccccagaggcgagctgtacagaagctaaacctacagaatcttgtgatcccacgggctccagaaaatctaacactcagcaatctgagtgaatcccagctagagctgagatggaaaagcagacatattaaagaacgctgtttacaatacttggtgcagtaccggagcaacagagatcgaagctggacggaactaatagtgaatcatgaacctagattctccctgcctagtgtggatgagctgaaacggtacacatttcgggttcggagccgctataacccaatctgtggaagttctcaacagtggagtaaatggagccagcctgtccactgggggagtcatactgtagaggagaatccttccttgtttgcactggaagctgtgcttatccctgttggcaccatggggttgattattaccctgatctttgtgtactgttggttggaacgaatgcctccaattccccccatcaagaatctagaggatctggttactgaataccaagggaacttttcggcctggagtggtgtgtctaaagggctgactgagagtctgcagccagactacagtgaacggttctgccacgtcagcgagattccccccaaaggaggggccctaggagaggggcctggaggttctccttgcagcctgcatagcccttactggcctcccccatgttattctctgaagccggaagcctgaacatcaatcctttgatggaacctcaaagtcctatagtcctaagtgacgctaacctcccctactcaccttggcaatctggatccaatgctcactgccttcccttggggctaagtttcgatttcctgtcccatgtaactgcttttctgttccatatgccctacttgagagtgtcccttgccctctttccctgcacaagccctcccatgcccagcctaacacctttccactttctttgaagagagtcttaccctgtagcccagggtggctgggagctcactatgtaggccaggttggcctccaactcacaggctatcctcccacctctgcctcataagagttggggttactggcatgcaccaccacacccagcatggtccttctcttttataggattctccctccctttttctacctatgattcaactgtttccaaatcaacaagaaataaagtttttaaccaatgatcatcaagaa

SEQ ID NO: 89 - DNA, 1608 bp, Mus musculus
cccagagaaagaagagcaagcaccatgttgaaactattattgtcacctagatccttcttagtccttcagctgctcctgctgagggcagggtggagctccaaggtcctcatgtccagtgcgaatgaagacatcaaagctgatttgatcctgacttctacagcccctgaacacctcagtgctcccactctgccccttccagaggttcagtgctttgtgttcaacatagagtacatgaattgcacttggaatagcagttctgagcctcaggcaaccaacctcacgctgcactataggtacaaggtatctgataataatacattccaggagtgcagtcactatttgttctccaaagagattacttctggctgtcagatacaaaaagaagatatccagctctaccagacatttgttgtccagctccaggacccccagaaaccccagaggcgagctgtacagaagctaaacctacagaatcttgtgatcccacgggctccagaaaatctaacactcagcaatctgagtgaatcccagctagagctgagatggaaaagcagacatattaaagaacgctgtttacaatacttggtgcagtaccggagcaacagagatcgaagctggacggaactaatagtgaatcatgaacctagattctccctgcctagtgtggatgagctgaaacggtacacatttcgggttcggagccgctataacccaatctgtggaagttctcaacagtggagtaaatggagccagcctgtccactgggggagtcatactgtagaggagaatccttccttgtttgcactggaagctgtgcttatccctgttggcaccatggggttgattattaccctgatctttgtgtactgttggttggaacgaatgcctccaattccccccatcaagaatctagaggatctggttactgaataccaagggaacttttcggcctggagtggtgtgtctaaagggctgactgagagtctgcagccagactacagtgaacggttctgccacgtcagcgagattccccccaaaggaggggccctaggagaggggcctggaggttctccttgcagcctgcatagcccttactggcctcccccatgttattctctgaagccggaagcctgaacatcaatcctttgatggaacctcaaagtcctatagtcctaagtgacgctaacctcccctactcaccttggcaatctggatccaatgctcactgccttcccttggggctaagtttcgatttcctgtcccatgtaactgcctttctgttccatatgccctacttgagagtgtcccttgccctctttccctgcacaagccctcccatgcccagcctaacacctttccactttctttgaagagagtcttaccctgtagcccagggtggctgggagctcactatgtagccaggttggcctccaactcacaggctatcctcccacctctgcctcataagagttggggttactggcatgcaccaccacacccagcatggtccttctcttttataggattctccctccctttttctacctatgattcaactgtttccaaatcaacaagaaataaagtttttaaccaatgaaaaaaaaaaaa

SEQ ID NO: 90 - protein, 369 aa, Mus musculus
MLKLLLSPRSFLVLQLLLLRAGWSSKVLMSSANEDIKADLILTSTAPEHLSAPTLPLPEVQCFVFNIEYMNCTWNSSSEPQATNLTLHYRYKVSDNNTFQECSHYLFSKEITSGCQIQKEDIQLYQTFVVQLQDPQKPQRRAVQKLNLQNLVIPRAPENLTLSNLSESQLELRWKSRHIKERCLQYLVQYRSNRDRSWTELIVNHEPRFSLPSVDELKRYTFRVRSRYNPICGSSQQWSKWSQPVHWGSHTVEENPSLFALEAVLIPVGTMGLIITLIFVYCWLERMPPIPPIKNLEDLVTEYQGNFSAWSGVSKGLTESLQPDYSERFCHVSEIPPKGGALGEGPGGSPCSLHSPYWPPPCYSLKPEA

SEQ ID NO: 91 - DNA, 978 bp, Mus musculus
actactccttcagggggaggacgagcttcggaggtctttttattgggctggcttaccattgggcacacacaaccccctcgccatgaatggtgtcgcagctgccctccttgtgtggattctgacttctccgagcagcagtgaccatggcagcgaaaatggttggcccaagcacacggcctgcaacagtgggggcttggaagtagtctaccagagctgtgatcccttacaggattttggcctttccattgaccagtgttccaagcagatccaatcaaatctcaacattagatttggcatcattctgagacaggatatcagaaagctgtttctggacataactctgatggcaaaaggctcttctattctgaactactcctatcccctttgtgaggaggaccagcccaagttctcattctgtggaagaagaaaaggagaacagatatactatgccggccctgtcaataaccctggacttgatgttccacagggagaatatcagctcttgctggaactgtacaatgaaaaccgtgctactgtggcttgtgccaatgccactgtcacctcctcctgagcatggtctgcaaggaaatgcacagtaaactcaatctcaggggaccccaaggtccctggactcacctagctgcaagaaccactgataaccaagagaggctttacaaagaaatttcttgtgggtcactcttccgatcttagctccagggacagatgttcccagacccaacagatgtaataaaccctcaaaaactatctatttctgaggaccctgagtagtcttgaagccctattgtagtacctctccttatgtaattacgtttcatcaaagctacttccttgcctgctccttagcaacacttccttgaagttgcctgggatgtaaaaaataaaataaaataaaataaaataaaataaaataaaataaaatgaaatgaaaaataaataaaataaaacaaaatctgccct

SEQ ID NO: 92 - DNA, 1282 bp, Mus musculus
aacagtctgctatctacaaaggtgaatcagagtcctgtgagaacacacaagcaaagcttctcctggaataaatcagctgacatctgatcctttccattgtctgtactgtttctgctgctctggaaaccatgcaaggacaggaacagaccaccatggcagtggttcctggagttgctgtgccttcaaagaattctgttatgacatcacaaatgtggaatgagaagaaagagaaattcttgaagggggaacccaaagtccttggggttttacaagtgatgattgctatcataaacctcagcttaggaataataattttgacaactttattttctgaactacccacttcagtgatgttaatggtcccaatttggggatcaataatgttcattgtctccggatccctgtccattgcagcaggagtgacacctacaaaatgcctgatcgttgccagtctaactctgaacactatcacctctgtgttggctgcaactgcaagcataatgggtgtagtcagtgtggctgtgggttcacagtttccgtttcggtataattatacaatcaccaagggtttggatgttttgatgttaattttcaatatgctagaattctgccttgctgtgtccgtctctgcatttggatgtgaagcttcctgttgtaactcccgtgaggttcttgtagtgctaccatcaaatcctgttgagacagtgatggcacccccaatgacacttcaaccattgctaccatcagaacaccaagggaccaatgttccaggaaatgtgtacaagaaccacccaggagaaatagtctaattttgatgtgtgtgtgtgtgtatttccctaggatattaacacttcattgcactggcttttgaggtgaatattagatttactgtaagtatgtaagtcaagcacttattaggtcaacaacacttcaacatattatattcattgtatgtacaaggggcaatgaatttgcaaagatgttttgaaagcaaacagaaaaaaaaaaaaaacaaccaaacaaaagacctcttagtgaaatgaggtctctttgcaaagactaaaaaactggagttcacatttttgggggtggggggggcttttgctaaataagtagatttagatgcttttgatcaagtacaaactcataaagtatgtaagaaattactaatgataggaaccaattttaatctcatatgtaacatagtgtataatttaatagatctggtaaaaatttataataaagagaaatgcctgaaaaaaaaaaaaaaaaaaaaaaaaaa

SEQ ID NO: 93 - protein, 226 aa, Mus musculus
MQGQEQTTMAVVPGVAVPSKNSVMTSQMWNEKKEKFLKGEPKVLGVLQVMIAIINLSLGIIILTTLFSELPTSVMLMVPIWGSIMFIVSGSLSIAAGVTPTKCLIVASLTLNTITSVLAATASIMGVVSVAVGSQFPFRYNYTITKGLDVLMLIFNMLEFCLAVSVSAFGCEASCCNSREVLVVLPSNPVETVMAPPMTLQPLLPSEHQGTNVPGNVYKNHPGEIV

SEQ ID NO: 94 - DNA, 722 bp, Mus musculus
agccagcgctcagttccacgggagccacaaagcagactgggaaacatgggtctggagcaacttgagtctcgactttgtctcctgctgctgctgggacatgtcctaatgcttgcctcatgccagccattgaccccctcccggtggtttgacatccagcatatctataacagagcctatccccgatgtgatgatgcaatgcgggccgttaacagttacacaggagtgtgtaaagacataaatacttttcttcatacaacttttgctaatgttgtccgtgtgtgtcataatccacgtaagatctgcaaaaatgggataagtagaaattgtcatgatagttcaaatcgggtacaagtaactatctgtatactcacaactccggccagtcattattccaactgcagataccgaacaacaagatcaatgaagtactacacagttgcctgtgaccccagaactcctcaggacagtcccatgtatccagtggttccagttcacttggatgggatattttagctccagtgccagcactttgtaccttcattcatcggctactgccaagatcatagttgggaagaaccgagtcctctgtttgcactgcatgtcagcatctgtcctcatgaaacctgttgctgactccctttaatttagctgaacaaaaagtttctaatcaataaacacttttgcacacaaaaaaaaaaaaaaaaaaaaaaaaaaaaaaaaaa

SEQ ID NO: 95 - protein, 155 aa, Mus musculus
MGLEQLESRLCLLLLLGHVLMLASCQPLTPSRWFDIQHIYNRAYPRCDDAMRAVNSYTGVCKDINTFLHTTFANVVRVCHNPRKICKNGISRNCHDSSNRVQVTICILTTPASHYSNCRYRTTRSMKYYTVACDPRTPQDSPMYPVVPVHLDGIF

SEQ ID NO: 96 - DNA, 1286 bp, Mus musculus
atgaagaaccttaagaattactcaatgttgaataaaccttctgtaatgaacactgagctaaaaactctcctggccattggaggatggaagtttggacctgccccgttcagtgccatggtctctactcctcagaaccgtcagatattcattcagtcagttatcagattccttcgtcaatataactttgatggcctcaacctggactggcagtaccctgggtctcgaggaagccctcctaaggacaaacatctcttcagtgttctggtgaaggaaatgcgtaaagcttttgaggaagaatctgtggagaaagacattccaaggctgctactcacttccacaggagcaggaatcattgacgtaatcaagtctgggtacaagatccctgaactgtctcagtctcttgactatattcaggtcatgacatatgatctccatgatcctaaggatggctacactggagaaaatagtcccctctataaatctccatatgacattggaaagagtgctgatctcaatgtggattcaatcatttcctactggaaggaccatggagcagcttctgagaagctcattgtgggatttccagcatatgggcatacctttatcctgagtgacccttctaagactggaattggtgcccctacaattagtactggcccaccaggaaagtacacagatgaatcaggactcctggcttactatgaggtttgtacatttctgaatgaaggagccactgaggtctgggatgccccccaggaagtaccctatgcctatcagggtaatgagtgggttggttatgacaatgtcaggagcttcaagttgaaggctcagtggctcaaggacaacaatttaggaggtgccgtggtctggcccctggacatggatgacttcagtggttctttctgtcaccagagacatttccctctgacatctactttaaagggagatctcaatatacacagtgcaagttgcaagggcccttattgagaggagctttacacaatgatttgtccttgaaactctcagaataagatcaagttcaacggtttttccacagtgcattctgcatcatgcttccatggagaataatagaaataagtcatgaactttcctaaattgaatcccagagtagtactaagatggatgtcttgtctgctgtaccagctgggaagaaacaaaaaatgctcttcatctgtcagctttggctaagctctgaacatcttttgcttcctgtaaaaccaccatgcttgtttcttgctctcacaataaattccacattcatagca

SEQ ID NO: 97 - protein, 328 aa, Mus musculus
MKNLKNYSMLNKPSVMNTELKTLLAIGGWKFGPAPFSAMVSTPQNRQIFIQSVIRFLRQYNFDGLNLDWQYPGSRGSPPKDKHLFSVLVKEMRKAFEEESVEKDIPRLLLTSTGAGIIDVIKSGYKIPELSQSLDYIQVMTYDLHDPKDGYTGENSPLYKSPYDIGKSADLNVDSIISYWKDHGAASEKLIVGFPAYGHTFILSDPSKTGIGAPTISTGPPGKYTDESGLLAYYEVCTFLNEGATEVWDAPQEVPYAYQGNEWVGYDNVRSFKLKAQWLKDNNLGGAVVWPLDMDDFSGSFCHQRHFPLTSTLKGDLNIHSASCKGPY

SEQ ID NO: 98 - DNA, 508 bp, Mus musculus
ttttataaaagaaagcatttaattgaggcttgctaacagtttcaggggttagttcatttacatcatggtagggagcatggtggtatgccagtggacatagtgctggagaaattacctagaattcagaagagatatagagacacagagtgacagagagactctagtcctgacatgaacttttgaaacctgaaagcctacccgtgctccagtgacacacacttcctacaacaaggacttgcctctgaatctttctgatcctgttaaagagttccactccctggtgactaagcattcaactatatgagcctatggggttcttattcaaatgaccacagtccccattcctgtcacctaaggctctgctgtaaatttatgtgcatctgagtaggtatgcatgtgcactcatgcacgtctgcctgtgttatcatgcatgtgactgtgatgtgcacatgtgcatgtgtatatgcttatgtgtatgtgagaagtcaactgagagtttaaggtggga

SEQ ID NO: 99 - DNA, 2237 bp, Mus musculus
ggagaagactactgtcttcaacacactagcctgagctaccttatccaagtgctccacatattccagaaggagaaggacagacttcaagttcaaatcacttccagagctgtgagtgacaagtgccttggcaggtagtgcacgcctcatcactgaaaggatccaaacaagactgccatggaagaaaatgaatactcaggatactgggaacctcctagaaagcgttgctgctgtgcaagacgtgggacacagctcatgttggtggggctgctgagcacagcaatgtgggctggcctgctggccctgcttcttctgtggcactgggaaacggagaagaatctaaaacagctgggagacactgcaattcagaatgtctctcatgttaccaaggacttacaaaaattccagagtaatcaattggcccagaagtcccaggttgttcagatgtcacaaaacttgcaagaactccaagctgaacagaagcaaatgaaagctcaggactctcggctctcccagaacctgaccggactccaggaggatctaaggaacgcccaatcccagaactcaaaactctcccagaacctgaacagactccaagacgatctagtcaacatcaaatccctgggcttgaatgagaagcgcacagcctccgattctctagagaaactccaggaagaggtggcaaagctgtggatagagatactgatttcaaagggaactgcatgcaacatatgtcccaagaactggctccatttccaacagaagtgctactattttggcaagggctccaagcagtggatccaggccaggttcgcctgcagtgacctgcaagggcgactagtcagcatccacagccaaaaggaacaggacttcctgatgcaacacatcaacaagaaggattcctggattggcctccaggatctcaatatggagggagagtttgtatggtcggacgggagccctgtgggttatagcaactggaatccaggggagcccaataacgggggccagggtgaggactgtgtgatgatgcggggatccggccagtggaacgacgccttctgccgcagctacttggatgcatgggtgtgtgagcagctggcaacatgtgagatatctgcccccttagcctctgtgactccaacaaggcccaccccaaaaagtgaaccctgacaaacttctgctcacactcttctggatttctcctctacctttatcgtggaaacagctgggccctgaggatacccctatcagggcccagggctctctgtgaccgaaggctttgattatgttcccacccatactgaagcagctggtggatgccagctcctgccagctacccagaaaccctctccagctctccagctaagctggccatcccattccatctgccttcctcaaacctgggccccagccttgctagctccctgactacgggcatgcatgtgggcagctgagccaaccagggagctgctgagaacaaagatttcgaaggcttcttttgcagtccccacctcctatcaagttccccactttctccccctcggcatcagagaacaggggttccctttccccaggatctgggatgagtcctcccatcaagtttgcatcagtggtcccaggactccgaccctccttggaggctaccaggtgtgctcctggtgcgggaggtattgaaggaactctaaacagctccagcaaggcgagcctggctctgtctggtaggcctggcccttctctcccattccttctaccttactaaaagctgttagagaacagtcctaaagctagcccccaaggtctattcccttatttggccacttcctcctcctgaggctgactacaaggtccagctatccaagtactgaagtctaacatcaaaagccccctttgtctcacctaagtagcaatgcccaatcaaaatacaccatcacatcatagcccagtctaacagaccgccctttttctcttcataaaattacacctgcaaccaggcgtagtggtgcaggcctttagtcccagcacttgggaggcagagacaagcgaatttctgagttcgaggccagcctggtctacaaagtgagttccaggacagccagggctacacagagaaaccctgtctcgaagaaagaaaaaaaaaaaaaattacacctgcgaggtcacttgggctgctgtttttctgcctgagtcagagggcagccacttaacttttcttccctgcttaataaaggatctctgtg

SEQ ID NO: 100 - protein, 331 aa, Mus musculus
MEENEYSGYWEPPRKRCCCARRGTQLMLVGLLSTAMWAGLLALLLLWHWETEKNLKQLGDTAIQNVSHVTKDLQKFQSNQLAQKSQVVQMSQNLQELQAEQKQMKAQDSRLSQNLTGLQEDLRNAQSQNSKLSQNLNRLQDDLVNIKSLGLNEKRTASDSLEKLQEEVAKLWIEILISKGTACNICPKNWLHFQQKCYYFGKGSKQWIQARFACSDLQGRLVSIHSQKEQDFLMQHINKKDSWIGLQDLNMEGEFVWSDGSPVGYSNWNPGEPNNGGQGEDCVMMRGSGQWNDAFCRSYLDAWVCEQLATCEISAPLASVTPTRPTPKSEP

SEQ ID NO: 101 - DNA, 1414 bp, Mus musculus
gttcgtggtctctcagcgttgttcgcagctcttctacctcgtacactatgtccggacgcggcaagaccggcggcaaggcccgcgccaaggccaagtcgcgctcttcacgcgccggccttcagttccctgtaggccgcgtacaccggctgctgcggaaaggccactacgccgagcgcgtgggcgcaggcgcgccggtgtacctggcagcggtgctcgagtacctcactgccgagatcctggagctggcgggcaacgcggcccgcgacaacaagaagacgcgcatcatcccgcgccacctgcagctggccatccgcaacgacgaggagctcaacaagctgctgggcggcgtgaccatcgcgcagggcggcgtcctgcccaacatccaggccgtgctgctgcccaagaagagcagcgccaccgtggggcccaaggcgccggcggtcggcaagaaggcctcgcaggcctctcaggagtactgagggggcccgcgccgaggccgcccagccctccccacacctccacaaaggcccttttaagggccaccacctccctcacagaaagagctgagccacttcgggctgcggcggcttgccgcgtccccatcctttctctttccctcctctcccctccccgccctcgcgccactcttccccttaccccgcacggcctccggttctgccacggaggacgcgggcgccgcggcacctgcccatcccggagctcgtgacatcgcttcgccgttctcggttgtggacgacggctccgtgcggagaggctgcggcgccttcccggagtctcggcccctcgctcagacgtccggccgcggtgggcttgaaggttagtccctccgcagctacagccccggcgtctttgcttcagcttggtgcttagccctcgcctggacctttgaaaagtgacttgattgccgggcttagaggaggagaaatggatcacattgtttccttcggtgtcaccgttgctggcctcataccagttgaccctgtcgccattcgtaggccagtgtggtgtggcggggccaaaaatggttgtaatgtgaacccagtttctctaggaatacctcccggcaagggtcagagagacgcttaccggcctgtggacaagagttctatacctgccgcaggcgactggcagccgggcctagccatccatcccctcttccccagcgactcaactacaacccaaacacctagataccagcacaagccggtgaatccctgtctggactgagcctctggcttctgaactggaacttttgcagctattacaagtttagaaccttaagtggggagatttaatgggctaattttattaaaggatcgtttggttttgtttttttaaaaaaaaaaaaaaaaaaaaaaaaaaaaaaaaaaaaaaaaaaaaaaaaaaaaaaaaa

SEQ ID NO: 102 - protein, 143 aa, Mus musculus
MSGRGKTGGKARAKAKSRSSRAGLQFPVGRVHRLLRKGHYAERVGAGAPVYLAAVLEYLTAEILELAGNAARDNKKTRIIPRHLQLAIRNDEELNKLLGGVTIAQGGVLPNIQAVLLPKKSSATVGPKAPAVGKKASQASQEY

SEQ ID NO: 103 - DNA, 1506 bp, Mus musculus
atggccaagctcattcttgtcacaggtctggcaattcttctgaacgtacagctgggatcttcctaccagctgatgtgctactataccagttgggctaaggacaggccaatagaagggagtttcaaacctggtaatattgacccctgcctgtgtactcacctgatctatgcctttgctggaatgcagaataatgagatcacttacacacatgagcaagacttgcgtgactatgaagcattgaatggtctgaaagacaagaacactgagctaaaaactctcctggccattggaggatggaagtttggacctgcctcgttcagtgccatggtctctactcctcagaaccgtcagatattcattcagtcagttatcagattccttcgtcaatataactttgatggcctcaacctggactggcagtaccctgggtctcgaggaagccctcctaaggacaaacatctcttcagtgttctggtgaaggaaatgcgtaaagcttttgaggaagaatctgtggagaaagacattccaaggctgctactcacttccacaggagcaggaatcattgacgtaatcaagtctgggtacaagatccctgaactgtctcagtctcttgactatattcaggtcatgacatatgatctccatgatcctaaggatggctacactggagaaaatagtcccctctataaatctccatatgacattggaaagagtgctgatctcaatgtggattcaatcatttcctactggaaggaccatggagcagcttctgagaagctcattgtgggatttccagcatatgggcatacctttatcctgagtgacccttctaagactggaattggtgcccctacaattagtactggcccaccaggaaagtacacagatgaatcaggactcctggcttactatgaggtttgtacatttctgaatgaaggagccactgaggtctgggatgccccccaggaagtaccctatgcctatcagggtaatgagtgggttggttatgacaatgtcaggagcttcaagttgaaggctcagtggctcaaggacaacaatttaggaggtgccgtggtctggcccctggacatggatgacttcagtggttctttctgtcaccagagacatttccctctgacatctactttaaagggagatctcaatatacacagtgcaagttgcaagggcccttattgagaggagctttacacaatgatttgtccttgaaactctcagaataagatcaagttcaacggtttttccacagcgcattctgcatcatgcttccatggagaataatagaaataagtcatgaactttcctaaattgaatcccagagtagtactaagatggatgtcttgtctgctgtaccagctgggaagaaacaaaaaatgctcttcatctgtcagctttggctaagctctgaacatcttttgcttcctgtaaaaccaccatgcttgtttcttgctctcacaataaattccacattcatagcaaaaaaaaaaa

SEQ ID NO: 104 - protein, 398 aa, Mus musculus
MAKLILVTGLAILLNVQLGSSYQLMCYYTSWAKDRPIEGSFKPGNIDPCLCTHLIYAFAGMQNNEITYTHEQDLRDYEALNGLKDKNTELKTLLAIGGWKFGPASFSAMVSTPQNRQIFIQSVIRFLRQYNFDGLNLDWQYPGSRGSPPKDKHLFSVLVKEMRKAFEEESVEKDIPRLLLTSTGAGIIDVIKSGYKIPELSQSLDYIQVMTYDLHDPKDGYTGENSPLYKSPYDIGKSADLNVDSIISYWKDHGAASEKLIVGFPAYGHTFILSDPSKTGIGAPTISTGPPGKYTDESGLLAYYEVCTFLNEGATEVWDAPQEVPYAYQGNEWVGYDNVRSFKLKAQWLKDNNLGGAVVWPLDMDDFSGSFCHQRHFPLTSTLKGDLNIHSASCKGPY

SEQ ID NO: 105 - DNA, 1209 bp, Mus musculus
atggccaagctcattcttgtcacaggtctggcaattcttctgaatgtacagctgggatcttcctaccagctgatgtgctactataccagctgggctaaggacaggccaacagaagggagtttcaaacctggtaatattgacccctgcctgtgtactcacctgatctatgcctttgctgggatgaagaataatgagatcacttacttaagtgagcaagacttgcgtgactatgaagcattaaatggtctgaaagacaggaacactgagctaaaaactctcctggccattggaggatggaagtttggacctgccccgttcagttccatggtctctactcctcagaaccgtcagacattcattaagtcagttatcagattccttcgtcaatataactttgatggcctcaacctggactggcagtaccctgggcctcgaggaagccctcctaaggacaaacatctcttcagtgttctggtgcaggaaatgcgtaaagcttttgaggaagaatccactttgaaccacattccaaggctgctactcacttccacaggagctggattcattgacgtaatcaagtctgggtacaagatccctgaactgtctcagtctctcgactatattcaggtcatgacatatgatctccatgatcctaagaatggctacactggagaaaatagtcccctctataaatctccatatgacattggaaagagtgctgatctcaatgtggattcaattattacctactggaaggaccatggagcagcttctgagaagctcattgtgggatttccagcatatggtcatacctttatcctgagtgacccttctaagaatggaataggtgaccctactgttagtgctggaccaccaggaaagtacacaaatgaacaaggactcctggcttactttgagatttgtacatttctgaatgaaggagccactgagatctttgatgccacccaggaagtaccctatgcctatctgggtaatgagtgggttggttatgacaatgtcaggagcttcaagttgaaggctcagtggctcaaggacaacaatttaggaggtgccgtggtctggcccctggacatggatgacttcagtggttctttctgtcaccagggacgtttccctctgacaactactttaaagagagatctgaatgtacacagtgcaagttgcaaggcctcttatcgaggggagctttag

SEQ ID NO: 106 - protein, 402 aa, Mus musculus
MAKLILVTGLAILLNVQLGSSYQLMCYYTSWAKDRPTEGSFKPGNIDPCLCTHLIYAFAGMKNNEITYLSEQDLRDYEALNGLKDRNTELKTLLAIGGWKFGPAPFSSMVSTPQNRQTFIKSVIRFLRQYNFDGLNLDWQYPGPRGSPPKDKHLFSVLVQEMRKAFEEESTLNHIPRLLLTSTGAGFIDVIKSGYKIPELSQSLDYIQVMTYDLHDPKNGYTGENSPLYKSPYDIGKSADLNVDSIITYWKDHGAASEKLIVGFPAYGHTFILSDPSKNGIGDPTVSAGPPGKYTNEQGLLAYFEICTFLNEGATEIFDATQEVPYAYLGNEWVGYDNVRSFKLKAQWLKDNNLGGAVVWPLDMDDFSGSFCHQGRFPLTTTLKRDLNVHSASCKASYRGEL

SEQ ID NO: 107 - DNA, 1198 bp, Mus musculus
tgattataaggggggaaaaagatgtgccctctgtcttttagggttacggccggtggagagcagctggacagcccgagcatatgcagcagcagcagccactggaacccagagagagcatgagctccaagccaaagtccttagagattatcggagcgcctttctcaaaaggacagcctcgaggaggggtagagaaaggccctgcagcactgaggaaagctggtcttctggaaaaacttaaagaaacagagtatgacgtgagagaccacggggacctggcctttgttgatgtccctaatgacagctcctttcaaattgtgaagaacccacggtctgtggggaaagccaatgaagagctggctggtgtggtggcagaggtccagaagaatggaagagtcagtgtggtgctgggtggagaccacagtctggcagttggaagcatctctggccacgccagggtccaccctgacctatgtgtcatttgggtggatgctcacactgacatcaacactcccctgacaaccagctctgggaatctgcatgggcaacctgtgtcctttctcctgaaggaactgaaaggaaagttcccagatgtaccaggattctcctgggtgactccctgcatatctgccaaagacatcgtgtacattggcttgcgagacgtagaccctggggaacactatataataaaaactctgggaattaagtatttctccatgactgaagtagacaagctggggattggcaaggtgatggaagagaccttcagctacctgctgggaaggaagaaaaggccgattcacctgagctttgatgtcgacgggctggacccagcattcaccccggcgaccggcaccccggttctgggaggcctatcttacagagaaggtctctacatcacagaagaaatttacaagacagggctcctttcaggactagatatcatggaagtgaacccaactcttgggaagacagcagaggaggtgaagagtactgtgaacacggcagtggctttaaccttggcttgtttcggaactcaacgggagggtaaccataagccagggactgactaccttaaaccacctaagtgactgtgaatgcgccacatgaaaaccatctggggcatcacagcaaagcagacagagctaagcaaacgccttctcctcccaagggcttgttcttttagaaaaaaaaaaaaaaa

SEQ ID NO: 108 - protein, 323 aa, Mus musculus
MSSKPKSLEIIGAPFSKGQPRGGVEKGPAALRKAGLLEKLKETEYDVRDHGDLAFVDVPNDSSFQIVKNPRSVGKANEELAGVVAEVQKNGRVSVVLGGDHSLAVGSISGHARVHPDLCVIWVDAHTDINTPLTTSSGNLHGQPVSFLLKELKGKFPDVPGFSWVTPCISAKDIVYIGLRDVDPGEHYIIKTLGIKYFSMTEVDKLGIGKVMEETFSYLLGRKKRPIHLSFDVDGLDPAFTPATGTPVLGGLSYREGLYITEEIYKTGLLSGLDIMEVNPTLGKTAEEVKSTVNTAVALTLACFGTQREGNHKPGTDYLKPPK

SEQ ID NO: 109 - DNA, 1433 bp, Mus musculus
tacggccggtggagaggagctggacagcccgagcacatgcagcagcagcagccgctggaacccagagagagcatgagctccaagccaaagtccttagagattatcggagcgcctttctcaaaaggacagcctcgaggaggggtagagaaaggccctgcagcactgaggaaagctggtctgctggaaaaacttaaagaaacagagtatgacgtgagagaccacggggacctggcctttgttgatgtccctaatgacagctcctttcaaattgtgaagaacccacggtctgtggggaaagccaatgaagagctggctggtgtggtggcagaggtccagaagaatggaagagtcagtgtggtgctgggtggagaccacagtctggcagttggaagcatctctggccacgccagggtccaccctgacctatgtgtcatttgggtggatgctcacactgacatcaacactcccctgacaaccagctctgggaatctgcatgggcaacctgtgtcctttctcctgaaggaactgaaaggaaagttcccagatgtaccaggattctcctgggtgactccctgcatatctgccaaagacatcgtgtacattggcttgcgagacgtagaccctggggaacactatataataaaaactctgggaattaagtatttctccatgactgaagtagacaagctggggattggcaaggtgatggaagagaccttcagctacctgctgggaaggaagaaaaggccgattcacctgagctttgatgtcgacgggctggacccagcattcaccccggcgaccggcaccccggttctgggaggcctatcttacagagaaggtctctacatcacagaagaaatttacaagacagggctcctttcaggactagatatcatggaagtgaacccaactcttgggaagacagcagaggaggtgaagagtactgtgaacacggcagtggctttaaccttggcttgcttcggaactcaacgggagggtaaccataagccagggactgactaccttaaaccacctaagtgactgtgaatgcgccacatgaaaaccatctggggcatcacagcaaagcagacagaactaagcaaacgccttctcctcccaagggcttgttcttttagaaaaaaaaaaaattttttttccagtaagtatgtatcccagcagttcctttctggtatgaaattcaggatgtggaaatcaaaacagctataaaactaggaaacatgtattccccattttagcataagttatccttaagaagtagtatccaattaattttcaattaaaaaatgcaccagcatttaaaatacacggtgatgttaagtgtcaattcacagttggaagcaaaggtatccagagaggtagcctatatccacgctgttcaaaagatgtataataaactctttataataaaa

SEQ ID NO: 110 - DNA, 1221 bp, Mus musculus
ctacaagggcaagaaatacaagcccctagacctgcgacccaagaagactagagccatgcgccgccggctcaccaagcacgaggaggagctggacagcccgagcacatgcagcagcagcagccgctggaacccagagagagcatgagctccaagccaaagtccttagagattatcggagcgcctttctcaaaaggacagcctcgaggaggggtagagaaaggccctgcagcactgaggaaagctggtctgctggaaaaacttaaagaaacagagtatgacgtgagagaccacggggacctggcctttgttgatgtccctaatgacagctcctttcaaattgtgaagaacccacggtctgtggggaaagccaatgaagagctggctggtgtggtggcagaggtccagaagaatggaagagtcagtgtggtgctgggtggagaccacagtctggcagttggaagcatctctggccacgccagggtccaccctgacctatgtgtcatttgggtggatgctcacactgacatcaacactcccctgacaaccagctctgggaatctgcatgggcaacctgtgtcctttctcctgaaggaactgaaaggaaagttcccagatgtaccaggattctcctgggtgactccctgcatatctgccaaagacatcgtgtacattggcttgcgagacgtagaccctggggaacactatataataaaaactctgggaattaagtatttctccatgactgaagtagacaagctggggattggcaaggtgatggaagagaccttcagctacctgctgggaaggaagaaaaggccgattcacctgagctttgatgtcgacgggctggacccagcattcaccccggcgaccggcaccccggttctgggaggcctatcttacagagaaggtctctacatcacagaagaaatttacaagacagggctcctttcaggactagatatcatggaagtgaacccaactcttgggaagacagcagaggaggtgaagagtactgtgaacacggcagtggctttaaccttggcttgcttcggaactcaacgggagggtaaccataagccagggactgactaccttaaaccacctaagtgactgtgaatgcgccacatgaaaaccatctggggcatcacagcaaagcagacagaactaagcaaacgccttctcctcccaagggcttgttcttttaaaaaaaaaaaaaaa

SEQ ID NO: 111 - protein, 323 aa, Mus musculus; identical to SEQ ID NO: 108

SEQ ID NO: 112 - DNA, 598 bp, Mus musculus
ggtacctaggtcagcaatcccatggcgtataaaagcatctcatctggccaggtcctggaacctttcctgagattctgccccaggatgccaactttgaataggatgaagactacaacttgttcccttctcatctgcatctccctgctccagctgatggtcccagtgaatactgatgagaccatagagattatcgtggagaataaggtcaaggaacttcttgccaatccagctaactatccctccactgtaacgaagactctctcttgcactagtgtcaagactatgaacagatgggcctcctgccctgctgggatgactgctactgggtgtgcttgtggctttgcctgtggatcttgggagatccagagtggagatacttgcaactgcctgtgcttactcgttgactggaccactgcccgctgctgccaactgtcctaagaatgaagaggtggagaacccagctttgatatgatgaatctaacaaaaactgcagtctcaatttggaaatctgactcatgtgcctttaaatgtgttcatattgcccatttaccctgcttcttgaaatgcttcttgaaaaataaagacaaatttgcatgtg

SEQ ID NO: 113 - protein, 138 aa, Mus musculus
MAYKSISSGQVLEPFLRFCPRMPTLNRMKTTTCSLLICISLLQLMVPVNTDETIEIIVENKVKELLANPANYPSTVTKTLSCTSVKTMNRWASCPAGMTATGCACGFACGSWEIQSGDTCNCLCLLVDWTTARCCQLS

SEQ ID NO: 114 - DNA, 1436 bp, Mus musculus
ttgtgtattatgtagccagggacagcattctgaactctaccctggcacttcctgaacctcctagcttataaataccagggcaggccagagaatcagggaggagtgagcaaaaattctcagaccagctgggcctgtcctccaggaggatgagaaactccaagactgccatttcattctttatccttgtggctgtccttgggtcccaggctggcctcatacaagaaatggaaaaagaagatcgtcgctataaccctccaataattcatcaaggctttcaagacacttcttcagactgctgcttctcttatgccacacagatcccatgtaaaagatttatatattatttccccaccagtggtgggtgcatcaagccgggcatcatctttatcagcaggaggggaacccaggtctgtgccgaccccagcgatcggagagttcagaggtgcctaagcaccctgaagcaaggcccaagatctgggaacaaggtcattgcttgagaaggagggcaggcattgtcacccactttcttctgtcttccccagtgaccgcctgcctaggagaccttgtttttatagatatttaaagcatttatccttctgttcaggtttagagcagtcaacagtattcatgtggactccgcctgacacggttagagccatctggagttgtaaacatcaagattgtctttgagtaattgttgggttttttttcggtttctcagcagattataaatggatacattattagggtagtctttggggctttggaatgtgtctggttctgatacaagcttaagccgggtaatatctagctgagatgaaatcaattttgccctaggccatacatatgtccagctttgtgggttcccagttgttcgccctgccacaatagagcaatgagtgcccccaataaagtccactccatgtagccacaggactgtctctttcttcagattcatagaactacggggccagtctgaaactgggctcttggggtgaaattatctcactcactcaccttgagtacaggagggagatgggaccatataactttgaatattctaaaccaaaagacatcatggtataatttttaaaaattaagaacacggtttattcctctgagcttggtgcaaaacagaggaataccttttccagcagggcgtcttcttctctgcctgacttttatctgcggaacctgaccttcacaccctctacctgaggaagttcaggtagttgttggcaaatctacaagtaagaacctgcacaccaagtatctgggattcctggaatgcctctccatgcaaatgaggcattcccagtactttaaacttcatctagtaatttacatttaccctgaaaactccttcctatactccaaggttcatgtatagccctggtttaccccaataaagtgtatgcacacaagctgtttcactg

SEQ ID NO: 115 - protein, 116 aa, Mus musculus
MRNSKTAISFFILVAVLGSQAGLIQEMEKEDRRYNPPIIHQGFQDTSSDCCFSYATQIPCKRFIYYFPTSGGCIKPGIIFISRRGTQVCADPSDRRVQRCLSTLKQGPRSGNKVIA

SEQ ID NO: 116 - DNA, 5085 bp, Mus musculus
gaccttggactgagcaaaggggcaacctggggacctggttgtattctttgcctttcccagtctccctcttctccctcattggaagatccactctgggccatgaggcttctcctgcttctggcttttatctctgtcatccctgtctctgttcagctattggacgcgaggcaatttttaatctataatgaagatcacaagcgctgcgtggacgctctaagtgccatctcagttcagacggcaacttgcaacccggaagctgaatcccagaaattccgctgggtgtcagattctcagatcatgagtgttgctttcaaattatgtttgggagtgccatcaaaaactgactgggcttccgtcaccctgtatgcctgtgattcgaaaagtgaatatcagaaatgggagtgtaagaatgacacactctttggaatcaagggcacagagttatattttaattatggcaacagacaagagaagaatatcaagctttacaaaggttcgggattgtggagcagatggaaggtctatggaaccacggatgacctgtgctcgagaggatatgaagccatgtactccttactgggcaatgcaaatggagccgtctgtgcatttccattcaagtttgaaaacaagtggtatgcagactgcacctctgccgggcgctcggacggatggctctggtgtggaaccaccactgactacgacaaagacaagctgtttggattttgtccattgcactttgagggaagcgagagattatggaacaaagatccactgactggcattctttaccagataaactccaagtctgctttaacctggcatcaggcaagggcaagctgcaagcagcagaatgctgacctcctgagtgtcacggagatccacgagcaaatgtacctcacaggattaaccagttccttgagctcgggactctggattggactcaacagtctgagtgtacgcagtggttggcagtgggctggaggaagcccattccggtatctgaactggctaccaggaagtccatcatcagagcctggaaagagctgtgtgtcactaaaccctggaaaaaatgccaagtgggaaaatctggaatgtgttcagaagcttggctacatttgtaaaaagggaaacaataccttgaacccatttatcattccctcagcaagcgatgtgcctaccggctgccctaatcagtggtggccctatgcaggccactgctacaggatccatagggaagagaagaagatccagaaatatgctttgcaagcttgtaggaaggagggtggggacctggcaagtatccacagcattgaggagtttgacttcatcttctcccagctcggatatgagccaaatgatgagctgtggattggtttaaatgacatcaagattcagatgtactttgagtggagtgatggaaccccagtgacatttactaaatggcttcctggagagccaagccatgagaacaacagacaggaggactgcgtggttatgaaaggcaaggatggatactgggcggacagagcctgtgagcaaccactaggttacatctgtaagatggtatcacaaagccatgctgtagtaccggagggtgcagacaaaggctgccggaaaggctggaaacggcatgggttttactgctacttgattggatccactctatccaccttcactgatgcaaaccacacatgcacaaatgaaaaggcttatttaacaacagttgaagacagatatgaacaagcattcctgactagtttggttggattgaggcctgaaaaatatttttggacaggactctcagatgttcaaaacaaagggacgtttcggtggactgtggacgagcaggtgcagtttacacactggaatgccgacatgccaggacgaaaggcgggatgtgttgccatgaaaaccggagtggcaggtggcttatgggatgttttgagttgtgaagaaaaggcaaaatttgtgtgcaaacattgggcagaaggagtgactcgcccaccagagcccacaacaactcctgaacccaaatgtccagaaaactggggtaccaccagtaaaaccagcatgtgtttcaaactgtatgcaaaaggaaagcatgaaaagaaaacgtggtttgaatctcgagatttttgcaaagctataggtggagagctggcgagcatcaagagtaaagatgaacagcaagtgatttggaggctgattacgagcagtggaagctaccatgagctgttttggttgggactgacctatggaagtccttcagaggggttcacctggagtgatggttctcccgtttcctatgaaaattgggcttacggtgaaccaaataattaccaaaatgttgaatattgtggtgagctgaaaggtgaccctggcatgtcctggaatgatatcaactgtgaacacctcaacaactggatttgtcagatacaaaaagggaaaacactactacctgagcccacacctgctccacaagacaatccaccagttactgcagatgggtgggttatttacaaagactaccagtactattttagcaaagagaaggaaaccatggacaacgcgcgacgattttgcaagaagaattttggtgatcttgctacaattaaaagtgaaagtgaaaagaagtttctatggaaatatataaacaagaatggtgggcagtcaccatattttattggcatgttaatcagcatggataagaaattcatttggatggatgggagcaaagtagattttgtggcttgggctacaggagaacccaactttgcaaatgatgatgaaaactgtgtaacgatgtacacaaattcagggttctggaatgacatcaactgtggttatccaaataacttcatctgccagagacataacagcagcatcaatgccactgccatgcctaccacacccacgacaccaggtggctgcaaggaaggttggcatttgtacaagaacaagtgctttaaaatttttggatttgctaatgaagaaaaaaaaagctggcaagacgcacggcaagcttgcaaaggactgaaaggaaacctggtgtccatagaaaatgcacaagagcaagcatttgttacctatcacatgagagactccactttcaatgcctggactgggctgaatgatatcaacgcagaacacatgttcctgtggacagctggacaaggagttcattatacaaactgggggaaaggctatcctggtggaagaagaagtagcctatcttatgaagatgctgactgtgtagttgtgattggtggcaattcacgagaggcagggacctggatggatgacacctgtgacagtaaacaaggctatatatgtcaaacacagactgacccttccctgcctgtttctccaaccactactccaaaagatggctttgttacatatgggaaaagcagctattcccttatgaaattgaagctaccatggcatgaagcagagacatattgcaaggatcatacttccctgcttgctagcattctagacccctacagtaatgcatttgcatggatgaaaatgcacccatttaatgtacccatatggattgccctgaacagcaacttgaccaataatgaatatacttggacggatagatggagggtgcggtacactaactggggtgctgacgagccgaagcttaagtcagcatgtgtttacatggatgttgatggctactggagaacatcatactgcaatgaaagtttttattttctctgcaaaaaatcagacgaaatccctgctactgaacctcctcaactgcctggcaagtgtccagagtcagaacagactgcgtggattcctttctatggccattgctactattttgaatcttcttttacgagaagttggggtcaggcttctctggaatgccttcgaatgggtgcctccctggtttccatcgagactgctgctgagtccagttttctgtcataccgtgttgaacctcttaaaagtaaaaccaatttttggataggcatgttccgaaatgttgaagggaagtggctttggttgaacgacaatcctgtctcctttgtcaactggaaaacaggcgatccctctggtgaacggaatgattgtgtagttctagcttcatcttcgggcctttggaataatatccactgttcttcgtacaaaggatttatttgtaaaatgccaaaaattattgatcctgtaactacacactcatccattacaaccaaagctgaccaaaggaagatggatcctcaacccaagggctcttctaaagcagcaggagtggtcaccgtggtcctcctgattgtgataggtgccggcgttgcagcctatttcttttataagaaaaggcatgcgttgcacatacctcaagaggccacctttgaaaacactctctacttcaacagtaatctgagtccaggaacaagtgacacgaaagatctcatgggcaacatcgagcagaatgagcatgcgatcatttagcatcctaatctgactgtctcatattcgaagttcaagaagtctgaactaaagttttcaattttttaattcaatatgattgtttctttttttaaaaaaaattagcactgggttgcattggtttgtcctttttcttcgcctaattgaacaataattgctcatttactatcctggcaagattagagaaacaaaaaacagaagagggataataatgttgattgttgattgccacttttgaagatacctttgatgaatacacagcactagcgtcttaacactgctacactggtggtaggtctcatgtcaaccctgcagatttcaagagttctagaaacaaccacgtaagcccactcagggtattttcaggacctcccagagatatgttatacaccgaattgtaattcaacatttaaaagtccatgttaaaatagacaagtccacataaccccagattgaataagaatgtattcaggtctttttcagcagtcctgattcatgtcagttattgagaactgtatttatccacatgtagaataataagctactgagaatcttgtttgtccccaggcaaggtttgacaggctgaatattgcaaatgtgttctgtgttctgttgtat

SEQ ID NO: 117 - protein, 1456 aa, Mus musculus
MRLLLLLAFISVIPVSVQLLDARQFLIYNEDHKRCVDALSAISVQTATCNPEAESQKFRWVSDSQIMSVAFKLCLGVPSKTDWASVTLYACDSKSEYQKWECKNDTLFGIKGTELYFNYGNRQEKNIKLYKGSGLWSRWKVYGTTDDLCSRGYEAMYSLLGNANGAVCAFPFKFENKWYADCTSAGRSDGWLWCGTTTDYDKDKLFGFCPLHFEGSERLWNKDPLTGILYQINSKSALTWHQARASCKQQNADLLSVTEIHEQMYLTGLTSSLSSGLWIGLNSLSVRSGWQWAGGSPFRYLNWLPGSPSSEPGKSCVSLNPGKNAKWENLECVQKLGYICKKGNNTLNPFIIPSASDVPTGCPNQWWPYAGHCYRIHREEKKIQKYALQACRKEGGDLASIHSIEEFDFIFSQLGYEPNDELWIGLNDIKIQMYFEWSDGTPVTFTKWLPGEPSHENNRQEDCVVMKGKDGYWADRACEQPLGYICKMVSQSHAVVPEGADKGCRKGWKRHGFYCYLIGSTLSTFTDANHTCTNEKAYLTTVEDRYEQAFLTSLVGLRPEKYFWTGLSDVQNKGTFRWTVDEQVQFTHWNADMPGRKAGCVAMKTGVAGGLWDVLSCEEKAKFVCKHWAEGVTRPPEPTTTPEPKCPENWGTTSKTSMCFKLYAKGKHEKKTWFESRDFCKAIGGELASIKSKDEQQVIWRLITSSGSYHELFWLGLTYGSPSEGFTWSDGSPVSYENWAYGEPNNYQNVEYCGELKGDPGMSWNDINCEHLNNWICQIQKGKTLLPEPTPAPQDNPPVTADGWVIYKDYQYYFSKEKETMDNARRFCKKNFGDLATIKSESEKKFLWKYINKNGGQSPYFIGMLISMDKKFIWMDGSKVDFVAWATGEPNFANDDENCVTMYTNSGFWNDINCGYPNNFICQRHNSSINATAMPTTPTTPGGCKEGWHLYKNKCFKIFGFANEEKKSWQDARQACKGLKGNLVSIENAQEQAFVTYHMRDSTFNAWTGLNDINAEHMFLWTAGQGVHYTNWGKGYPGGRRSSLSYEDADCVVVIGGNSREAGTWMDDTCDSKQGYICQTQTDPSLPVSPTTTPKDGFVTYGKSSYSLMKLKLPWHEAETYCKDHTSLLASILDPYSNAFAWMKMHPFNVPIWIALNSNLTNNEYTWTDRWRVRYTNWGADEPKLKSACVYMDVDGYWRTSYCNESFYFLCKKSDEIPATEPPQLPGKCPESEQTAWIPFYGHCYYFESSFTRSWGQASLECLRMGASLVSIETAAESSFLSYRVEPLKSKTNFWIGMFRNVEGKWLWLNDNPVSFVNWKTGDPSGERNDCVVLASSSGLWNNIHCSSYKGFICKMPKIIDPVTTHSSITTKADQRKMDPQPKGSSKAAGVVTVVLLIVIGAGVAAYFFYKKRHALHIPQEATFENTLYFNSNLSPGTSDTKDLMGNIEQNEHAII

SEQ ID NO: 118 - DNA, 2949 bp, Mus musculus
ggtcgttcttctgcgctgcgccaggagctgcaagcacaggcatccatcgcccctgcttcgtccggcatcccggtgagcgggtccaagggccaggggtatagtcctgcggggcctctacgctgtggggtgcgggaccggggcatcacgcgcagcctccctgccgggagcgctcatcggacccgcccgggaacttgggatgcgctggactctgacgatgcagacctcgctgaccttggtggcgtccagggaaagtctcgggggtcccaagtggtttgcaggtccctggcggccggtgtttaaatctcctctgaccccgctcttccctgcagcccgctgccgccgccatgcccttctccaacagccataatacgcagaagctgcgcttcccggccgaggatgagttccctgatctgagcagccacaacaaccatatggccaaggtgctgaccccggagctgtacgccgagctccgtgccaagtgcacgccgagcggctttactttggacgacgccattcagactggcgtagacaatccgggtatgcacacccctgtagcgtcaggcttccgcctccccaagaagccccccgggcaaggatcccactgctcttccctgaaccttcggtgggctggggtcccctgttcccctctccgcgcttagccttaagaggcctcggcttggctcctgcctgacggtgacgtcactgtcgccgcgcccctcctccaggccacccgtacatcatgactgtgggtgcagtggcgggcgacgaggagagttacgacgtattcaaggacctcttcgaccccattgttgaggagcggcacggcggctaccagcccagtgatgagcacaagaccgacctcaacccagacaacctgcaggtgcggggaatcagggtccgggcgtgctggggagaggggtctcggcgctcactctagccaccaccttgtattcccagggtggcgatgacctggaccccaactacgtgctgagctcgcgagtgcgcacaggccgcagcatccgcggcttctgtctccccccgccactgcagccgcggggagcgcccgcgcccatcgagaagctggcagtagaaggtaggggtcgggtaacagcctccagaggctgctgcgctcttctctgcgcgcgcgttcccactggggactgagggtcgaagacccaccttcgcccgcggggacacactgctgcagggcggggcggcgggcttgctcgcccgcccaccctgtccttgaactctgcactccgcagctctgtccagcctagatggcgacctgtctggcaggtactacgcgctcaagagcatgactgaggcggagcagcagcagctcattgacgaccacttcctcttcgataagcctgtgtcgcctctgctgctggcctccggcatggcccgggactggccggatgctcgtggcatatggtacgagccccccccccccccagtccttagaaggtggggcctgccctgaattcttagtttgtgcagtgcctccctccgcccaggtgaccttggttctgcggatgactgtggtccttgcgctgcgggaggccgcagtctccagggactcaagggtggtgaccagtctctttggcgtctgttctccgccctcctcctgggagccgggcgcttcttgttttctctccaccttctcacccccttattccgccgggatcgtgccaggtgccaatgacgcaaaagcctccgcacccgtccgggcagggctcctacccctgcagactgagcgggcggaggcgtgcttcctctggtgggatgctctggaggctccagacccttgcgggccacacagcacatgactggtgattgagatgtatatgagctgccttcctccactgttgctgggattggcgtcttggtgacagctcacattccagcactttgttgaggaagcattttgtttgaggtcacccacctatagtgtctttcttgggtgtgatctaaggccacattctggcagggactccagcctgactttacccctcttctaggcacaatgacaataagactttcctggtgtggattaacgaggaggaccacctgcgagtcatctccatgcagaaggggggcaacatgaaggaagtgttcacccgattctgcaccggcctcactcaggtcaggcctgggcattccccagggccttcctcactaggggcgttggtgctggggagggatcaggggcagatcagagcccacatctcccagggtccgtagggcttccggccaggcttccttctcttaactctcccttctccaccttcagatcgaaactctcttcaagtccaagaactatgagttcatgtggaatcctcacctgggctacatcctcacatgcccatccaacctgggcaccggactgcgggcaggtgtgcacatcaagctgccccacctggggaagcacgagaagttctcggaggtgctcaagcggctgcggcttcagaagcgaggcacaggtgaggggcagaaggcacaggtgaggggcagaaggtgcaggtgttgtcctcagccccgctgacctgcccatgtcccaccgcaggtggcgtggacaccgctgctgtcggtggggtgtttgatgtctccaacgctgaccgcctgggcttctcggaggtggaactggtgcagatggtggtggacggagtgaaactactcattgagatggagcaacggctggagcagggtcaggcaatcgatgacctcatgccggcccagaagtgaagcctggccctcgccaccatcaggctgccgcttcctaacttattaaccgggcagtgcccgccatgcatccttgatgtttgccgcctggcggctgagcccttagcctcgctgtagagacttctgtcgccctgggtagagtttatttttttgatggctaagctgttgctgacactgaaaataaactagggtttggcctgccct

SEQ ID NO: 119 - DNA, 814 bp, Mus musculus
ggattaacgaggaggaccacctgcgagtcatctccatgcagaaggggggcaacatgaaggaagtgttcacccgattctgcaccggcctcactcagatcgaaactctcttcaagtccaagaactatgagttcatgtggaatcctcacctgggctacatcctcacatgcccatccaacctgggcaccggactgcgggcaggtgtgcacatcaagctgccccacctggggaagcacgagaagttctcggaggtgctcaagcggctgcggcttcagaagcgaggcacaggtggcgtggacaccgctgctgtcggtggggtgtttgatgtctccaacgctgaccgcctgggcttctcggaggtggaactggtgcagatggtggtggacggagtgaaactactcattgagatggagcaacggctcgagcagggtcaggcaatcgatgacctcatgccggcccagaagtgaagcctggccctcgccaccatcaggctgccgcttcctaacttattacccgggcagtgcccgccatgcatccttgatgtttgccgcctggcggctgagcccttagcctcgctgtagagacttctgtcgccctgggtagagtcacttctgatggctaagctgttgctgacacttgagaataaactagggtttggcctgccctagaacacaccacacaaagacagtcaacaaacgtgggcgtggtcgcttacaaataccccgcaaggcgcccagtttgtgcgctccgcaggttctgcgcacaaggcggcccataaggcaccaggttcttaacctcaggctggccggtataaaac

SEQ ID NO: 120 - DNA, 1433 bp, Mus musculus
gtcgttcttctgcgctgcgccaggagctgcaagcacaggcatccatcgcccctgcttcgtccggcatcccgcccgctgccgccgccatgcccttctccaacagccataatacgcagaagctgcgcttcccggccgaggatgagttccctgatctgagcagccacaacaaccatatggccaaggtgctgaccccggagctgtacgccgagctccgtgccaagtgcacgccgagcggctttactttggacgacgccattcagactggcgtagacaatccgggccacccgtacatcatgactgtgggtgcagtggcgggcgacgaggagagttacgacgtattcaaggacctcttcgaccccattattgaggagcggcacggcggctaccagcccagtgatgagcacaagaccgacctcaacccagacaacctgcagggtggcgatgacctggaccccaactacgtgctgagctcgcgagtgcgcacaggccgcagcatccgcggcttctgtctccccccgcactgcagccgcggggagcgccgcgccatcgagaagctggcagtagaagctctgtccagcctagatggcgacctgtctggcaggtactacgcgctcaagagcatgactgaggcggagcagcagcagctcattgacgaccacttcctcttcgataagcctgtgtcgcctctgctgctggcctccggcatggcccgggactggccggatgctcgtggcatatggcacaatgacaataagactttcctggtgtggattaacgaggaggaccacctgcgagtcatctccatgcagaaggggggcaacatgaaggaagtgttcacccgattctgcaccggcctcactcagatcgaaactctcttcaagtccaagaactatgagttcatgtggaatcctcacctgggctacatcctcacatgcccatccaacctgggcaccggactgcgggcaggtgtgcacatcaagctgccccacctggggaagcacgagaagttctcggaggtgctcaagcggctgcggcttcagaagcgaggcacaggtggcgtggacaccgctgctgtcggtggggtgtttgatgtctccaacgctgaccgcctgggcttctcggaggtggaactggtgcagatggtggtggacggagtgaaactactcattgagatggagcaacggctggagcagggtcaggcaatcgatgacctcatgccggcccagaagtgaagcctggccctcgccaccatcaggctgccgcttcctaacttattaaccgggcagtgcccgccatgcatccttgatgtttgccgcctggcggctgagcccttagcctcgctgtagagacttctgtcgccctgggtagagtttatttttttgatggctaagctgttgctgacactgaaaataaactagggtttggcctgccct

SEQ ID NO: 121 - DNA, 1402 bp, Mus musculus
gcacaggcatccatcgcccctgcttcgtccggcatcccgcccgctgccgccgccatgcccttctccaacagccataatacgcagaagctgcgcttcccggccgaggatgagttccctgatctgagcagccacaacaaccatatggccaaggtgctgaccccggagctgtacgccgagctccgtgccaagtgcacgccgagcggctttactttggacgacgccattcagactggcgtagacaatccgggccacccgtacatcatgactgtgggtgcagtggcgggcgacgaggagagttacgacgtattcaaggacctcttcgaccccattattgaggagcggcacggcggctaccagcccagtgatgagcacaagaccgacctcaacccagacaacctgcagggtggcgatgacctggaccccaactacgtgctgagctcgcgagtgcgcacaggccgcagcatccgcggcttctgtctccccccgcactgcagccgcggggagcgccgcgccatcgagaagctggcagtagaagctctgtccagcctagatggcgacctgtctggcaggtactacgcgctcaagagcatgactgaggcggagcagcagcagctcattgacgaccacttcctcttcgataagcctgtgtcgcctctgctgctggcctccggcatggcccgggactggccggatgctcgtggcatatggcacaatgacaataagactttcctggtgtggattaacgaggaggaccacctgcgagtcatctccatgcagaaggggggcaacatgaaggaagtgttcacccgattctgcaccggcctcactcagatcgaaactctcttcaagtccaagaactatgagttcatgtggaatcctcacctgggctacatcctcacatgcccatccaacctgggcaccggactgcgggcaggtgtgcacatcaagctgccccacctggggaagcacgagaagttctcggaggtgctcaagcggctgcggcttcagaagcgaggcacaggtggcgtggacaccgctgctgtcggtggggtgtttgatgtctccaacgctgaccgcctgggcttctcggaggtggaactggtgcagatggtggtggacggagtgaaactactcattgagatggagcaacggctggagcagggtcaggcaatcgatgacctcatgccggcccagaagtgaagcctggccctcgccaccatcaggctgccgcttcctaacttattaaccgggcagtgcccgccatgcatccttgatgtttgccgcctggcggctgagcccttagcctcgctgtagagacttctgtcgccctgggtagagtttatttttttgatggctaagctgttgctgacactgaaaataaactagggtttggcctgcccta

SEQ ID NO: 122 - protein, 381 aa, Mus musculus
MPFSNSHNTQKLRFPAEDEFPDLSSHNNHMAKVLTPELYAELRAKCTPSGFTLDDAIQTGVDNPGHPYIMTVGAVAGDEESYDVFKDLFDPIIEERHGGYQPSDEHKTDLNPDNLQGGDDLDPNYVLSSRVRTGRSIRGFCLPPHCSRGERRAIEKLAVEALSSLDGDLSGRYYALKSMTEAEQQQLIDDHFLFDKPVSPLLLASGMARDWPDARGIWHNDNKTFLVWINEEDHLRVISMQKGGNMKEVFTRFCTGLTQIETLFKSKNYEFMWNPHLGYILTCPSNLGTGLRAGVHIKLPHLGKHEKFSEVLKRLRLQKRGTGGVDTAAVGGVFDVSNADRLGFSEVELVQMVVDGVKLLIEMEQRLEQGQAIDDLMPAQK

SEQ ID NO: 123 - DNA, 2646 bp, Mus musculus
cacttcctcttttttcactctgaagataggaactgccccaaggcagtcagaagcgatgggcagattccaggctgtggtagacctgcttcacccgctggccagggagtgctgagggcaccagcctattgcctccctccctgcttctggggtaactgaggaccaccttgcaggctagcaggatggagcgagcctgtgagaagcaggactcagtgtgcaacctggtggctgtgtttgagaacaacaggactccgggagaagcacctggatcccatagccttgaggaccagccccatagccctgaacaccagctgtccctgtccccagagccttgggaggcaccccctgtcaaggaggccttgaagtctgaattccggccagtgagcaggacatatctgagttccctcaagaacaagttatcgagtggggcttggaggagatcctgtcagcctggggtcagcccggggccagagacacaggaacctgaggagaagagggtcgtgcgagagcttctggagacggagcaggcctatgtggctcgcctgcacctgcttgaccaggccactgtgttcttccaggagctgctgagggaggcaggccgcagcaaggccttccctgaggacgtggtgaagctcattttctccaacatctcctccatctatcgtttccacgcccagttcttccttcccgagctgcagcggcgcgtggatgactgggcagccacgccccgcattggggatgtgatccagaagctggccccgtttctgaaaatgtacagcgagtacgtgaagaactttgagcgggccgcggaactgctggccacgtggatggacaagtctcagcccttccaggaggtggtcacccgcatccagtgcagcgaggcctccagcagcctgaccctgcagcaccacatgttggagcctgtgcaaagaatcccgcggtacgaactgctgctcaaggaatatgtgcagaagctgccagcccaggccccagacctcgaagatgcccagagagcactggacatgatcttctcagctgcacagcactccaatgcagccattgcagagatggagcggctgcagggcctgtgggatgtgtaccagcgcctgggcttggaggatgacatcgtggacccctccaacaccctgctccgagagggccctgttctcaagatctctttccgccgcagcgacccaatggaacgctacctggttttgttcaacaacatgcttctgtattgtgtaccccgagtcctccaagtgggtgcccagttccaggtgcggactcgcatcgatgtggccggcatgaaggtgcgggagctgaccgatgctgagttcccacactccttcctggtgtccggaaagcagcgcacactggagctgcaggcccggtcccgagatgaaatggtttcctggatgcaggcctgccaggcagccattgaccaggttgagaagcggagtgagaccttcaaggctgctgtccagggacctcagggggacacgcaggagcccaagccacaggtagaggagctgggtctccgagcgcctcagtgggtccgggacaagatggtgaccatgtgcatgcgctgccaggagcccttcaatgccctgactcgtcggcgccaccactgccgggcttgcggctatgtagtgtgtgccaagtgctctgactaccgtgcggagctgaaatatgacagcaacaggcccaaccgagtctgcctgacctgctacacatttctcactggaaacgtactccctcaaggcaaggaggacaagaggcggggcatcttggagaaagaggcctcagcagcacccgagcagagtctggtgtgcagcttcctgcagctcataggggacaagtgcagcaggagccttccccggagctggtgtgtgatcccccgggatgacccccttgtcctgtatgtctatgcagccccccaggacacaaaggctcacacctccatccccctactgggctatcaggtgatttcagggccccagggggaccctcgggttttccagctgcaacagtcaggccagcagtacaccttcaaggccgagtctgtggagctgcagggccgctgggtgacagctatcaagcgtgcggccagtggccggacccctgagggacctgacgaagaagatgtgtctgactgaaccacagccagctgctcatcgagagtccatgacagccttcccaggcctaccccatggttctatcacttcaccccaagctgggctacccctgacccacctgatgtctataccgagacaattcatcctgactctgtatccagcagacacaggccctttccttcaggaagataccaccaagtgtcccagctggctggagagggggcggatatccctggatcatgaaatcttcattgctaaaacaatgttgtagcttaatgggtggcatgcccatctaacatggatgaagccttgggttcagtcctcagcgaaacataaactgggcatggtaatacatgcaacagttgggtaggtagaggaagaaggaccaaaaggccgaggtcgccctggctatagagtgagttacagtccagcctgggctacacaagaacctgagagcacaaaaaaagtcaaaataaaagatgacaagtaccaataactgggactgttgtcaccc

SEQ ID NO: 124 - DNA, 420 bp, Mus musculus
gggtctgcccactaagaagatgaagccttttcatactgccctctccttcctcattcttacaactgctcttggaatctgggcccagatcacacatgcaacagagacaaaagaagtccagagcagtctgaaggcacagcaagggcttgaaattgaaatgtttcacatgggctttcaagactcttcagattgctgcctgtcctataactcacggattcagtgttcaagatttataggttattttcccaccagtggtgggtgtaccaggccgggcatcatctttatcagcaagagggggttccaggtctgtgccaaccccagtgatcggagagttcagagatgcattgaaagattggagcaaaactcacaaccacggacctacaaacaataacatttgcttgaagagaagggtgtgaactgcca

SEQ ID NO: 125 - protein, 122 aa, Mus musculus
MKPFHTALSFLILTTALGIWAQITHATETKEVQSSLKAQQGLEIEMFHMGFQDSSDCCLSYNSRIQCSRFIGYFPTSGGCTRPGIIFISKRGFQVCANPSDRRVQRCIERLEQNSQPRTYKQ

SEQ ID NO: 126 - DNA, 3008 bp, Mus musculus
actgcacactgttccctctcttagaactagcatgttgggtgttatgtagtcaaaggagggcattatgagctgtaccccagggacttcctgatcctcttacatgtataaatagcaagaccgggccaggaacagcaagcagtctgaaggccagctgggtctgcccactaagaagatgaagccttttcatactgccctctccttcctcattcttacaactgctcttggaatctgggcccagatcacacatgcaacagagacaaaagaagtccagagcagtctgaaggcacagcaagggcttgaaattgaaatgtttcacatgggctttcaagactcttcagattgctgcctgtcctataactcacggattcagtgttcaagatttataggttattttcccaccagtggtgggtgtaccaggccgggcatcatctttatcagcaagagggggttccaggtctgtgccaaccccagtgatcggagagttcagagatgcattgaaagattggagcaaaactcacaaccacggacctacaaacaataacatttgcttgaagagaagggtgtgaactgccagctactttctttggtcttccccagtgaccacctaagtggctctaagtgtttatttttataggtatataaacattttttttttctgttccactttaaagtggcatatctggctttgtcacagaggggaaacttgtctgtgccaaccccagtcatctgaaaactcagatgcctggaaggtctgaagctgacctcaatgactacacataatatttgattgagataaatgggcaaggtctggagagatggcttggtggttaagagcacctgctgctcttccagaggacctgggttcaattcccacttagatggcagctcaaactatctataattccaattccaaagaaaactgatgccctattttgccctttagttagtagtatttacagtattctttataaattcaccttgacatgaccatcttgagctacagccatcctaactgcctcagaatcactcaagttcttccactcggtttcccagcggattttaagtggataaactgtgagagtggtctgtgggactttggaatgtgtctggttctgatagtcacttatggcaacccaggtacattcaactaggatgaaataaattctgccttagcccagtagtatgtctgtgtttgtaaggacccagctgattttcccaccacccctccatcagtaagccactaataaagtgcatctatgcagccacaggtctgtctgcctcttttgcttcagtttcctaggactatgggctgaaattgggctgttagggagaaagcatctcactcgcttttattgaatctgcagtggaaaagaaacagagggagtcaggtaactttgaatattttcttcaaaacaaaagatatcatggtacaatttttttttaattttttgtttgtttgtttttgtttttcgagacagggtttctctgtgtagccctggctgtcctggaactcactctgtagaccaggttggcctccaactcagaaatccgcctgcctctgcctcccgagtgctgggattaaaggcgtgcgccaccaccacccggcccatggtacaatttttaaatttccagaaatatagtttattccaatgtagacttcatatcaaggatgtattttacccactatagagagaatcattaaagtgatctacaaatctttggaagttctccctgttcgataagatcctcaattctattcgaggatctcaacttggtcagcttgtttttataccagtctcatgctgtttttttactgtggctctgtatgataatcccttcagcagtgtctctattgttcaggggtgtttgggttacctaagatcttttgtgtttccatatgaattttaagattgttattttcaaaatctgtgaagaattgcattggaattttgatgggaattgcaatgaatctataaattttttttgataagctgaccattgtcaaaatattaaactagaccatgagcataggtggtctttccatcttctgggtcttctttgattgttttagagttttcattgtataggccttttgctttattcattaggtttattccaagatattattttgcaggtattgagagtgggattttcctcccaatttcttcctcagtatgtttgtcatttgcttataggaagactattggtttttttgtatgtgcagatcgtgtcctgacacttggctgaaagtgtttatcagctctacgagttttctagtgtagcatttagggccttttatagagagagaaagaatgatatcatctgccaatgaatattgcttgacttcttcctttcctgtttgaatccatcttgtctccttctcttgccttactgctgtagcaaaaacttcaagtactcagctgaaaagaagtactgagagaaagtatccatgtccctttcctgatttttagcagaaatgcttccagtttttctccggttagcattccgttggctacaggcttgttgtatatttcctttattgtattgaaatacgttccttgtatttctgttgtcttcagggctttaataatgaagagctgttggttttcaccacaggcattttctgaatctactttctgctttcttgaattgagtccatttatacagtggatctcatttactgatttatgtatgttgaacatccctgcaagtctggaatgaagctctttatgatttcagaaaacagatttttcttagtcctcatttgtaacctctccccctagcctgaaacctggctgctcaggtttcactgttagcaggaagagagcgtggggtggacctaccgccctatcgttctgccactcccactgcggctgcctgccacctagctgttcctgagccaacacgtggtcacctgcaactggactcctaggatgatttggcgggaatgggcccctccccctttttataacccagtgtctggaatagtaaaattgaaccttggtcag

SEQ ID NO: 127 - protein, 122 aa, Mus musculus; identical to SEQ ID NO: 125

SEQ ID NO: 128 - DNA, 984 bp, Mus musculus
gctggacctagcccggaagaagctggtgtcagcagcatgaagctgaatttcagtggcctgagggccctggtgacaggggcagggaaagggattggacgggacactgtgaaggccctgcacgcctcaggagccaaagtggtggcggtgactcggaccaactcagacctggtcagccttgccaaagagtgtcccggaatagagcccgtgtgtgtggacctgggtgactgggatgccacagagaaggcgctgggcggtatcggccctgtggacttactggtgaacaatgcagcgctggttataatgcagcccttcctggaagttaccaaggaagcctttgacaggtccttcagtgtaaatcttcgctctgtgtttcaggtatcccagatggtagccagggacatgattaaccgtggcgtgccagggtccattgtcaatgtctccagcatggtggcccatgtcacctttcctaacttgatcacctacagctccaccaagggagcgatgactatgctgaccaaagccatggccatggaactagggccacacaagatccgggtaaactccgtaaaccccactgtggtgctgactgacatgggcaagaaagtctctgcagaccctgagtttgccaggaagctcaaggagcgccacccactgaggaagttcgcagaggttgaagacgtcgtcaacagcatcctcttcctgctcagcgacagaagtgcctctaccagtggcggtggcatcctggtggatgctggctacctggcctcctagtctgctcagttgccgcggacatctgagtggccttcttagccccaccctcagccaaagcatttactgatctcgtgactccgccctcatgctacagccacgcccaccacgcagctcacagttccacccccatgttactgtcgatcccacaaccactccaggcgcagaccttgttctctttgtccactttgttgggctcatttgcctaaataaacg

SEQ ID NO: 129 - protein, 244 aa, Mus musculus
MKLNFSGLRALVTGAGKGIGRDTVKALHASGAKVVAVTRTNSDLVSLAKECPGIEPVCVDLGDWDATEKALGGIGPVDLLVNNAALVIMQPFLEVTKEAFDRSFSVNLRSVFQVSQMVARDMINRGVPGSIVNVSSMVAHVTFPNLITYSSTKGAMTMLTKAMAMELGPHKIRVNSVNPTVVLTDMGKKVSADPEFARKLKERHPLRKFAEVEDVVNSILFLLSDRSASTSGGGILVDAGYLAS

SEQ ID NO: 130 - DNA, 1286 bp, Mus musculus
gacttcctggtcttggcgggctggcagcaggcaggcttcagcaggcgagcagcagcaagagtaaaaggtgaccgcggctgcccaccccagagccatggggcgggcacgagatgccatcctggacgctcttgaaaacttgtcaggggatgaactcaaaaagttcaagatgaagctgctgacagtgcaactgcgagaaggctatgggcgcatcccacgcggggccctgctgcagatggacgccatagatctcactgacaaacttgtcagctactatctggagtcgtatggcttggagctcacaatgactgtgcttagagacatgggcttacaggagctggctgagcagctgcaaacgactaaagaagagtctggagctgtggcagctgcagccagtgtccctgctcagagtacagccagaacaggacactttgtggaccagcacaggcaagcactcattgccagggtcacagaagtggacggagtgctggatgctttgcatggcagtgtgctgactgaaggacagtaccaggcagttcgtgcagagaccaccagccaagacaagatgagggagctcttcagctttgttccatcctggaacctgacctgcaaggactccctcctccaggccttgaaggaaatacatccctacttggtgatggacctggagcagagctgaggtatcttttccagctacattatctagctcctgactttgtatacacaatttttgaaaaaaacaatttgtatttgtgtttactctctgggatgtttttgctgagctaagctacatttccaaaggccatatgtggcccagtggtagaacatataccaaacatgcacaaatcagtctccaacacctaaattccacagacccaagttatggcttctccattctagtgcaaaggcttctcaaatgattcctggctccccatttgggagcttttcccatattatcccaaaaggctacctttgagaacacatgtaacattatccaggtcaagctaaaaagcgagagggggggatgggaaccaggtaatggttaatcccagcaaccaggaggcagaggaggctgttccagaacagccaagggtacagagagaaaccctatctcaaaaaaacaaaaacaaaaaaaaaacaggcaatggggcttagaaaggcacctagaaacaaaccagtccgtagaagactttgggctctgggaactcctgtctagacaaggctggcctaactcaagagatctgcccagctctgcctcctgagttatgagattaaatgcatgtatcacc

SEQ ID NO: 131 - protein, 193 aa, Mus musculus
MGRARDAILDALENLSGDELKKFKMKLLTVQLREGYGRIPRGALLQMDAIDLTDKLVSYYLESYGLELTMTVLRDMGLQELAEQLQTTKEESGAVAAAASVPAQSTARTGHFVDQHRQALIARVTEVDGVLDALHGSVLTEGQYQAVRAETTSQDKMRELFSFVPSWNLTCKDSLLQALKEIHPYLVMDLEQS

SEQ ID NO: 132 - DNA, 3451 bp, Mus musculus
ggcaacagtcctaacattcacctctcgtgtgtttgtgtctgttcgccatcccgtctccgctcgtcacttatccttcactttccagaggggtccccccgcagaccccggtgaccctcaggtcggccgactgcggcagctggcgcccgaacagggaccctcggataagtgacccttgtctctatttctactatttggtgttcgtcttgtattgtctctttcttgtctggctatcatcacaagagcggaacggactcaccacagggagctgcagtcccgcctacggaggagaggaatttttggccagatggaaaatatctcacgtcacggggattccttacaatccccagggacaggccattgttgaacaaacacaccaaaatataaaggcacagcttaataaacttcaaaaggctggaaaatactatacaccccaccatctattggcacatgctctttttgtgctgaatcatgtaaatatggacaatcaaggccatacggcggccgaaagacattggggtccaatttcagccgatccaaaacctatggttatgtgaaaagatcttctcacagggtcctggaaaggacccgatgtcctaataacagccggacgaggctatgcttgtgtttttccacaagatgccaaaatcaccaatttgggtccctgaccggttcatccgacctttcactgagcggaagggatcgacgcccacgcctagcgctgcggaaaaaacgccgccgcgagatgagaaagatcaccaaaaaagtccggagaatgaccctagaccccatcaaagagaagacggcttagcaacagctgcaggcgttgatctccgaagcggaggaggttcttaaaacctcacaaactccccaaacctctttgaccttatttcttgctttgttgtctgtcctcggccccctgcctgtgaccggggaaagttattgggcttacctacctaaaccacctattctccatcccgtgggatgggaaagtacagaccccattagagttctgacaaatcaaaccatgtatttgggtggttcgcctgactttcacgggtttagaaacatgtctggtaatgtacattttgaggagaagtctgatacgctccccatttgcttttccttctccttttctacccccatgggctgctttcaagtagataagcaggtatttctttctgatacacccacggttgataataataaacctggggaaaagggtgataaaaggcgtatgtgggaactttggttgactaccttggggaactcgggggccaatacaaaactggtccctataaaaaagaagttgccccccaaatatcctcactgccagatcgcctttaagaaggacgccttctgggagagagacgagtctgctcctccacggtggttgccttgcgccttccctgaccagggggtgagtttttctccaaaaggggcccttgggttactttgggatttctcccttccctcgcctagtgtagatcagtcagatcagattaaaaacaaaaaggatctatttggaaattatactccccctgtcaataaagaggttcatcgatggtatgaagcaagatgggtagaacctacttggttctgaaaaaattctcctaaagatcccaatgatagagattttactgctctagttccccatacaaaattgtttcgcttagttgcagcctcaagacatcttattctcaaaaggccaagatttcaagaacatgagatgattcctacatctgcctgtgttacttacccttatgccatattattaggattacctcagctaatagatatagaaaaaagaggatctacttttcatatttcctgttcttcttgtagattgactaattgtttagattcttctgcctacgactatgcagcgatcatagtcaagaggccgccatatgtgctgctacctgtagatattggtgatgaaccatggtttgattattctgccattcaaacttttaggtatgccacagatttaattcgagctaagcgatttgtcgcggccattattctgggcatatctgctttgattgctattattacttcctttgctgtagctactactgccttagttaaagagatgcaaactgctacgtttgttaataattttcataggaatgttacattagccttatctgaacaaagaataatagatttaaaattagaagctagacttaatgctttaaaagaagtagttttagagttgggtcaagatgtggcaaatttaaagaccagaatgtccactaagtgtcatgcaaattatgattttatctgcgttacaccactaccatataatgcttctgagagctgggaaagaaccaaggctcatttactgggcatttaaaataacaatgagatttcatataacatacaaaaattaaccaacctaattagtgatatgagcaaacaacatattgacgcagtggaccttagtggcttggctcagtcttttgccaacggagtgaaggctttaaatccattggattggacacaatatttcatttttataggagttggagccctgcttttggtcatagtacttatgattttccccattgttttccagtgctttgcaaagagccttgaccaagtgcagtcagatcttaacgtgcttcttttaaaaaagaaaaaagggggaaatgccgcgcctgcagcagaaatggttgaactcccgagagtgtcctacacttaggagagaagcagcccaaggggttgtttcccaccaaggacgacccgtctgcgtgcacgcggatgagcccatcagacaaagacatactcattctctgctgcaaacttggcatagctctgctttgcctggggctattgggggaagttgcggttcgtgctcgcagggctctcacccttgattcttttaataactcttctgtgcaagattacaatctaaacgattcggagaactcgaccttcctcctggggcaaggaccacagccaacttcctcttacaagccacaccgactttgtccttcagaaatagaaataagaatgcttgctaaaaattatatttttaccaataagaccaacccaataggtcgattattaatcactatgttaagaaatgaatcgttaccttttagtactatttttactcaaattcaaaggttagaaatgggaatagaaaatagaaagagacgctcaacctcagttgaagaacaggtgcaaggactaagggcctcaggcctagaagtaaaaaggggaaagaggagtgcacttgtcaaaataggagacagatggtggcaaccagggacttataggggaccttacatctacagaccaacagatgccccgctaccatatacaggaagatatgatttaaattttgataggtgggtcacagtcaatggctataaagtgttatacagatccctcccctttcgtgaaaggctcgccagagctagacctccttggtgtgtgttaaccc

SEQ ID NO: 133 - DNA, 1357 bp, Mus musculus
gttaagatctgactgcttttggtcatagtacttatgattttccccattgttttccagtgctttgcaaagagccttgaccaagtgcagtcagatcttaacgtgcttcttttaaaaaagaaaaaagggggaaatgccgcgcctgcagcagaaatggttgaactcccgagagtgtcctacacttaggagagaagcagccaaggggttgtttcccaccaaggacgacccgtctgcgtgcacgcggatgagcccatcagacaaagacatactcattctctgctgcaaacttggcatagctctgctttgcctggggctattgggggaagttgcggttcgtgctcgcagggctctcacccttgattcttttaataactcttctgtgcaagattacaatctaaacgattcggagaactcgaccttcctcctggggcaaggaccacagccaacttcctcttacaagccacaccgactttgtccttcagaaatagaaataagaatgcttgctaaaaattatatttttaccaataagaccaacccaataggtcgattattaatcactatgttaagaaatgaatcgttaccttttagtactatttttactcaaattcaaaggttagaaatgggaatagaaaatagaaagagacgctcaacctcagttgaagaacaggtgcaaggactaagggcctcaggcctagaagtaaaaaggggaaagaggagtgcacttgtcaaaataggagacagatggtggcaaccagggacttataggggaccttacatctacagaccaacagatgccccgctaccatatacaggaagatatgatttaaattttgataggtgggtcacagtcaatggctataaagtgttatacagatccctcccctttcgtgaaaggctcgccagagctagacctccttggtgtgtgttaactcaaaaagaaaaagacgacatgaaacaacaggtacatgattatatttatctaagaacaagaataaacgtttggaaaaagatttttcattataccaaggagggggcagtggctagacaattagaacacatttctgcggatacctttgacataaggtataataaataacctttatgagcccaaccttgcggttcccagggttcaaataagttcttggtcacaaactgttcttaaaacaaagatgtgagacaagtggtttcctgacttggtttggtatcaaatgttttgatctaagctctgagtgttctattctcctatgttcttttagaacttatccaagtcttatgtaaatgcttatgtaaaccatgatataaaagagtgctgattttttgagtaaacttgcaacagtcctaaaaaaaaaaaaaaaaaaaa

SEQ ID NO: 134 - protein, 320 aa, Mus musculus
MPRLQQKWLNSRECPTLRREAAKGLFPTKDDPSACTRMSPSDKDILILCCKLGIALLCLGLLGEVAVRARRALTLDSFNNSSVQDYNLNDSENSTFLLGQGPQPTSSYKPHRLCPSEIEIRMLAKNYIFTNKTNPIGRLLITMLRNESLPFSTIFTQIQRLEMGIENRKRRSTSVEEQVQGLRASGLEVKRGKRSALVKIGDRWWQPGTYRGPYIYRPTDAPLPYTGRYDLNFDRWVTVNGYKVLYRSLPFRERLARARPPWCVLTQKEKDDMKQQVHDYIYLRTRINVWKKIFHYTKEGAVARQLEHISADTFDIRYNK

SEQ ID NO: 135 - DNA, 467 bp, Mus musculus
cacacacgtgtcctgaagctgtctactttctaccatgtctggacgcggcaagcagggtggcaaggctcgcgccaaggccaagacccgctcctcccgggccggcctgcagttccccgtgggccgcgtgcaccggctgctccgcaagggcaactactcggagcgcgtgggcgccggcgccccggtctacctggcggccgtgctggagtacctgacggccgagatcctggagctggcgggcaacgcggcccgcgacaacaagaagacgcgcatcatcccgcgccacctgcagctggccatccgcaacgacgaggagctcaacaagctgctgggccgcgtgaccatcgcgcagggcggcgtcctgcccaacatccaggccgtgctgctgcccaagaagaccgagagccaccacaaggccaagggaaaataagaccagccgttcactcacccgaaaagaaaaaaaaaaaagg

SEQ ID NO: 136 - protein, 130 aa, Mus musculus
MSGRGKQGGKARAKAKTRSSRAGLQFPVGRVHRLLRKGNYSERVGAGAPVYLAAVLEYLTAEILELAGNAARDNKKTRIIPRHLQLAIRNDEELNKLLGRVTIAQGGVLPNIQAVLLPKKTESHHKAKGK

SEQ ID NO: 137 - DNA, 465 bp, Mus musculus
ttctgcatgagggaagctgtctactttctaccatgtctggacgcggcaagcagggtggcaaggctcgcgccaaggccaagacccgctcctcccgggccggcctgcagttccccgtgggccgcgtgcaccggctgctccgcaagggcaactactcggagcgcgtgggcgccggcgccccggtctacctggcggccgtgctggagtacctgacggccgagatcctggagctggcgggcaacgcggcccgcgacaacaagaagacgcgcatcatcccgcgccacctgcagctggccatccgcaacgacgaggagctcaacaagctgctgggccgcgtgaccatcgcgcagggcggcgtcctgcccaacatccaggccgtgctgctgcccaagaagaccgagagccaccacaaggccaagggaaaataagaccagccgttcactcacccgaaaagaaaaaaaaaaaagg

SEQ ID NO: 138 - protein, 130 aa, Mus musculus; identical to SEQ ID NO: 136

SEQ ID NO: 139 - DNA, 2069 bp, Mus musculus
gccccccgccgcctgctgggccggccgaggatgcagcgcagcgcctcggtggccaggctcgctctgctccagcgtgcttgctaacttccccggctccgtctctgcctgccggggtcgccccgtgtccctgcgccctccgggtctggttctctaagctctctgggcgctgcctccgggtcccttgcagcccgctcgcgagcccgcgctcccctcctgcgccccaccctcgtcctcgccatgctgcccttcggcctcgtggccgccctgctgctggccgcagggcctcggccgagcctgggcgacgaagccatccactgccccccttgctccgaggagaagctggcgcgctgccgcccccccgtgggttgcgaggagttggtgcgggagccaggctgcggttgttgcgccacttgcgccctgggcttggggatgccctgcggggtgtacaccccacgctgtggctcaggcatgcgctgctaccctccccggggggtggagaagcccctgcgtacattgatgcacgggcaaggcgtgtgcacggagctgtcggaaatcgaagccatccaggaaagcctgcagacctctgacaaggatgagagcgaacatcccaacaacagcttcaacccctgcagtgcccatgatcacaggtgcctgcagaagcatatggccaaaattagagatcggagcaagatgaagatcgtggggacacctcgggaggaaccccgtcctgtgccccagggttcctgccagagcgagctgcaccgggccctggagcggctggccgcctcacagagccgtacccacgaagacctcttcatcatccccattccaaactgtgaccgcaacggcaacttccaccccaaacagtgtcaccccgccctggacggacagcgtggcaagtgctggtgtgtggatcggaagacaggggtgaagcttcctgggggtttggaacccaagggggagctggactgccaccagctggctgacagcttccaagagtgagacctgccagcaggtgggggactcagtgccccctactaggcccctatgctctggggttgtagagttgacctggcttggagtctgagtcctggctgtctgcctctggccctgcccaaaatttccctttgtgtgtgtgtgcacacgcgtgcacatgctcgtgtgtgtgtgtttataggatgtgccctacaggataagcctgagccttctcgtgcccatgatgggcttagatacccccaagaggtctgagtgtagcaataggaattcccagtgtgctttcaaatcactatggaattcattcattcattcattcattcatccacctgaaaacacgtattgaaaacatactatgtgccagctttagttttcagcgccgggaagcctcgttgtgatttcctccgattgtagaatgtgacgaggtgttctcaactcaaggcccacgggagaagagaaacctcattcttggagtgagagagaaagaaagtggtgaacattgaacgccctccctctccatcttggccagagcgtaggaaggtgtagagtagaggctcacggctcctgagacccacctctgctctgtgctgctgcgtagagctctctccttggggcctccagggcacaagcttgagggacagggcccatctttggtcagggtattcggtcatccgacaatgccttgcaggtagatctctgctgtgggaaaggaatggggtgaggaaggccggaaggacagggggcagggaggtgggctcatcactctgagcctgcaggagagaggaaagaatgtgagattggattgtgtgtgtctgaggaatggccccattctggtccctatttgtggttattggtcatgaagtcaccaggatgactctatctttctagttgcccaccacctgtggctttgcctccgtccccatatttccctacccttatacctcctatcccgccatctctacccccctgggcattttctggcttgcctggatagatttagagacctgcttgttggctataatgtcttttcattctttcttcttccttttttaaaaaaatcagaacaaaaccaaaaa

SEQ ID NO: 140 - protein, 254 aa, Mus musculus
MLPFGLVAALLLAAGPRPSLGDEAIHCPPCSEEKLARCRPPVGCEELVREPGCGCCATCALGLGMPCGVYTPRCGSGMRCYPPRGVEKPLRTLMHGQGVCTELSEIEAIQESLQTSDKDESEHPNNSFNPCSAHDHRCLQKHMAKIRDRSKMKIVGTPREEPRPVPQGSCQSELHRALERLAASQSRTHEDLFIIPIPNCDRNGNFHPKQCHPALDGQRGKCWCVDRKTGVKLPGGLEPKGELDCHQLADSFQE

SEQ ID NO: 141 - DNA, 574 bp, Mus musculus
gtttttacaacattttactattaaagtaacatgtgacctttatagaaaaataaaaaaaaaaattgaagttccttataaagaagcatcatctttgacaggttggtacaagtctggtttatcatcaccgagatgatgcatcctaacattaattgttctccttatcttagcgtgaattataagcagttcccacgttgctgcagtctcttaacggcagtaagttgattaattttagtggcagtgtggatcgatagcagaagagtcttgagttgggaagctgtaagttatgggagtcattagtacagtcaaccattgggaaccaatctcgtagtgtctgttggtttcaggattcacagaagtgactctggatgttttctttaatatctacagcatcccttggaccaggagacactagatcctggcctttctggcttgcttttcccaatgccaagggagactaaatgtggttctggtaatcatgctctgtttcttcatccagggcttcgggatgcttgagaagtgagtaggtgatcgtattctcagcctcagttttggcagcttcttccagatcaggagg

SEQ ID NO: 142 - DNA, 1415 bp, Mus musculus
gttctctgggtactatctgccaagccgggagggaagcctgtgcctgcagctgactcgctccagagctgatgggaatcctgccgttcctactgatccccatggagagcaactggactgtccatgtgttctcacggactttgtgccatatgctactgtggacagccgtgctaaatcttgctgctgggactcatgatcttccaaaggctgtggtcaaactcgagcccccgtggatccaggtgctcaaggaagacacggtgacactgacatgcgaagggacccacaaccctgggaactcttctacccagtggttccacaatgggaggtccatccggagccaggtccaagccagctacacgtttaaggccacagtcaatgacagtggagaatatcggtgtcaaatggagcagacccgcctcagcgaccctgtagatctgggagtgatttctgactggctgctgctccagacccctcagctggtgtttctggaaggggaaaccatcacgctaaggtgccatagctggaggaacaaactactgaacaggatctcgttcttccataatgaaaaatccgtgaggtatcatcactacagtagtaatttctctatcccaaaagccaaccacagtcacagtggggactactactgcaaaggaagtctaggaaggacactgcaccagtccaagcctgtcaccatcactgtccaagggcccaagtccagcaggtctttaccagtattgacaattgtggctgctgtcactgggattgctgtcgcagccattgttattatcctagtatccttggtctatctcaagaaaaagcaggttccagacaatcctcctgatctggaagaagctgccaaaactgaggctgagaatacgatcacctactcacttctcaagcatcccgaagccctggatgaagaaacagagcatgattaccagaaccacatttagtctcccttggcattgggaaaagcaagccagaaaggccaggatctagtgtctcctggtccaagggatgctgtagatattaaagaaaacatccagagtcacttctgtgagtcctgaaaccaacagacactacgagattggttcccaatggttgactgtactaatgactcccataacttacagcttcccaactcaagactcttctgctatcgatccacactgccactaaaattaatcaacttactgccgttaagagactgcagcaacgtgggaactgcttataattcacgctaagataaggagaacaattaatgttaggatgcatcatctcggtgatgataaaccagacttgtaccaacctgtcaaagatgatgcttctttataaggaacttcaattttttttttatttttctataaaggtcacatgttactttaatagtaaaatgttgtaaaaacaaaaaaaaaaaa

SEQ ID NO: 143 - protein, 293 aa, Mus musculus
MGILPFLLIPMESNWTVHVFSRTLCHMLLWTAVLNLAAGTHDLPKAVVKLEPPWIQVLKEDTVTLTCEGTHNPGNSSTQWFHNGRSIRSQVQASYTFKATVNDSGEYRCQMEQTRLSDPVDLGVISDWLLLQTPQLVFLEGETITLRCHSWRNKLLNRISFFHNEKSVRYHHYSSNFSIPKANHSHSGDYYCKGSLGRTLHQSKPVTITVQGPKSSRSLPVLTIVAAVTGIAVAAIVIILVSLVYLKKKQVPDNPPDLEEAAKTEAENTITYSLLKHPEALDEETEHDYQNHI

SEQ ID NO: 144 - DNA, 2113 bp, Mus musculus
ggtgcaccggattccagctgttttcgcctgctcctcgccgtctccgccgctgccctcgttcgccatgctctccgtccgcaccccgctcgccaccatcgctgaccagcagcagctgcagttgtcgccgctgaagcgactcaccctggctgacaaggagaacacgcccccgactctcagcagcacccgcgtcctggccagcaaagctgcgaggagaatcttccaggactccgccgagctggaaagtaaagcgcctactaaccccagcgttgaggatgagccgttactgagagaaaacccccgccgcttcgttgtctttcccatcgagtaccatgatatctggcagatgtacaagaaagccgaggcctccttttggactgccgaggaggtggacctttccaaggatattcagcactgggaagctctgaaacccgatgagagacattttatatctcacgttctggctttctttgcagcgagtgatggcatagtcaatgagaacttggtggagcgatttagccaagaagttcaagttacagaggcccgctgtttctatggcttccaaattgccatggaaaacatacactctgaaatgtacagtctccttattgacacttacattaaagatcccaaggaaagagaatatctcttcaatgctattgaaactatgccttgtgtgaagaagaaggctgactgggccttgcgctggattggggacaaagaggctacgtatggagaacgcgttgtggcctttgccgccgtagaaggaatcttcttttccggttcttttgcatcgatattctggctcaagaaacgggggctgatgccgggccttacattttccaatgagcttattagcagagacgagggtttacactgtgactttgcctgcctgatgttcaagcacctggtacacaagccagcggagcagagggtccgagagataatcaccaacgccgttaggatagagcaggagttcctcacggaggccttgcccgtgaagctcatcgggatgaactgcactttgatgaagcagtacattgagtttgtggccgacaggcttatgctggagctgggttttaacaagattttcagagtagaaaatccgtttgacttcatggaaaatatctcactagaaggaaagacaaacttctttgagaagcgagtaggcgagtatcagaggatgggagtcatgtcgaattcgacagagaactcttttaccttggatgctgacttctaagtaactgatcgtgtgctcttcgctgatttttgtccccttgccattaaaagaaaccagcaaaaacaaccaactggctacaccatgaattgtcattaaatttgctaaacaggtgtctaaaaagctgtgtagctacctcagtcctgtttgccaggctggtcactagaagaaagtatacttcaaacaatgggtacttggatccttagggagatcctgtccttggcttttacaagtagtgtggtcacctttgacctcatcaaagtactaacagcactgggccaggttttaggagcagtgaccatcaagcaagcaggtttaaacatttagatgctgtttagggctgtttaaagatgtcggactgcttcctgcaggcatgcagagtctacttaacaagtttgtaaataaaattggcactttgcacacacacacatagtactgtcaggcgattaaactatacattttatgaggtagtacctctatgcttttttttattttaatgctcagtattatcttgaagtttgcaaatgctatgatggtacagtaaattctgacatttgccctaataagagtgtcactttttttttttttcttcgagacagagtttctctgtatagccctggctgtacggaattcacaagtgagtttgagcccagtggtgggtacacccgtgggactctcttacaaaccaaaacaggaaaagcaagtgttccctgaggtagtttactgtgatctagcttcctcatgaactgacataaccctgatcagtttccttgattattgtataaatgtttttgtaatatgaaaagcctttgtaccttttaaattattgttacttaaaattaataaactcttgaattaacagtcttgaactttcatggc

SEQ ID NO: 145 - protein, 390 aa, Mus musculus
MLSVRTPLATIADQQQLQLSPLKRLTLADKENTPPTLSSTRVLASKAARRIFQDSAELESKAPTNPSVEDEPLLRENPRRFVVFPIEYHDIWQMYKKAEASFWTAEEVDLSKDIQHWEALKPDERHFISHVLAFFAASDGIVNENLVERFSQEVQVTEARCFYGFQIAMENIHSEMYSLLIDTYIKDPKEREYLFNAIETMPCVKKKADWALRWIGDKEATYGERVVAFAAVEGIFFSGSFASIFWLKKRGLMPGLTFSNELISRDEGLHCDFACLMFKHLVHKPAEQRVREIITNAVRIEQEFLTEALPVKLIGMNCTLMKQYIEFVADRLMLELGFNKIFRVENPFDFMENISLEGKTNFFEKRVGEYQRMGVMSNSTENSFTLDADF

SEQ ID NO: 146 - DNA, 1878 bp, Mus musculus
gctctgagtctgcgcgacgcccggaattcccacggttctgcagtcaccgcggcgatcacccgcccagtcttctgtagcggacacggggtgcagaatgacctggagagtggctgtgcttctcagcctggtgctgggtgctggtgccgttcccgtcggtgtggacgatcccgaggatggaggcaagcactgggtggtgattgtggcgggctccaatggctggtataattaccgacaccaggcagacgcatgccacgcctaccagatcatccaccggaacgggattcctgacgagcagatcatagtgatgatgtatgacgacattgccaactctgaagaaaaccctaccccaggtgttgtgatcaaccgacctaacggcacagatgtatacaagggagtcctgaaggactacaccggagaggatgtgactccagagaatttcctcgccgtgctgagaggtgacgcagaagctgtgaagggcaaagggtctggaaaagtcttgaagagtggcccccgagatcatgtcttcatttacttcaccgaccacggagccaccgggatcctggtgtttcctaatgatgatcttcatgtcaaggacctgaataagactattcgctacatgtatgaacacaaaatgtaccagaagatggtgttctacattgaagcttgtgagtctggctccatgatgaaccacctgcccgacgacatcaacgtttatgcaactactgcggccaaccccaaggagtcatcttatgcctgctactacgacgaggagaggggcacttacctgggtgactggtacagcgtcaactggatggaagactccgatgtggaggacctgaccaaagagacccttcacaagcagtaccacctggtcaagtcccacaccaacaccagccatgtcatgcaatatgggaacaaatctatctctaccatgaaagtgatgcagtttcagggaatgaagcacagagccagttcccccatctccctgcctccggtcacacaccttgacctcacccccagccctgacgtgcccctgaccatcttgaagaggaagctgctgagaaccaacgacgtgaaggaatcccagaatctcattgggcagatccagcaatttctggatgccaggcacgtcattgagaagtctgtgcacaagatcgtttccctgctggcgggatttggggaaactgctgagagacatctgtcagagaggaccatgctcacagcacatgactgctaccaggaggctgtaacccacttccgcacacactgctttaactggcactctgtcacgtacgagcatgccttgcggtacttgtatgtgctggccaatctctgtgaggcaccatatccgattgacaggatagagatggccatggacaaagtgtgtcttagtcactactgaacagctcgcttcccaatgagtgagcacagtccactggaatatgaaccaaccagagactgaaagggcggagccagaggcagcacccgcgcccctggcccccaggaatactgcccgcccaccccagggcttgctttttgaagatacctgcttactaagaagccagtttgggtgggtaaagctctctggaagaaggaactttgcttcttaggagtttttttgtttgtttgttttggtttgttttgttgtccattagctttcaagagcaaattccccgcggcttctctagccagggaaggaatcgtctgagaaattcaaagctgaaacctcttgccgtcttcacagtgatttcactgaagaagagggtggaaagcaagcccctatgggagaattatttttagaattatataatttttgattgcttttatattttattctgtaataatggatgtcttaaaacaaataagtgaagtgagaca

SEQ ID NO: 147 - DNA, 3635 bp, Mus musculus
ccggcctgcactggccgccagccaccgagaggaggagcagaggatcctcggagcgcaataaaacggcggctcggcccgagcccgcagcaaacacagccatagaaggcagcggaggagccgagccgggctgcgctcgctcgccgccccccagcctctttcttctccgccgggtgcactgccctgcgccgtcccctcgccgctgcgccccttgacaaagaggacagaaagtttgcgcgggggagcgggccaggtgaggaggggcgtgcccggcgccccagtccgcgccccagcagccggacccaggcccccagcgcgcccgccatgggcgccgcggcccgcagcctccggctggcgctcggcctcctgctgctagccacgctgctgcgcccggccgacgcctgcagctgctccccggtgcacccgcaacaggcgttttgcaatgcagacgtagtgatcagagccaaagcagtgagcgagaaggaggtggattccgggaatgacatctatggcaaccccatcaagaggattcagtatgagatcaagcagataaagatgttcaaaggacctgacaaagacatcgagtttatctacacggccccctcttcagcagtgtgcggggtctcgctggacgttggaggaaagaaggagtatctaattgcaggaaaggcagaaggagatggcaagatgcacattaccctctgtgacttcattgtgccctgggacacgcttagcatcacccagaagaagagcctgaaccacaggtaccagatgggctgtgagtgcaagatcactcgctgtcccatgatcccttgctacatctcctccccggatgagtgcctctggatggactgggtcacagagaagagcatcaatgggcaccaggccaagttcttcgcctgcatcaagagaagtgatggttcttgcgcgtggtaccgcggggcggcaccccccaagcaagagtttcttgacatcgaggacccgtaagaaggctgacagagcccctgtggccaattgaaaagcctctgagggtttagactggtccagctttgacatcccttcctggaaacagcatgaataaaacatcaatcatccaagtgggttcacgctagtgtgattctgccccctcccctattttccctagacatggtagtgggtctggagggacaggcgggccaggttccctgccataccccttccctctgccagcctgagcactgtgtgtctcagtctttgatccttgctacaggcaggagtggagcacagacttgttaccaggtctctctggcactgtcacatgcagcagacaggcagcattaagggtaccctagctctgttagggcagagcctgggaatgtgcattttgcagaaactcttgaaggttgttgtaagactgtgtagccggcctaccaggtccttttcatcctgagagtgacatgtccctcgttttctgcagtggccacctctctctctggcccttgcaaatgcttcccatccctcctgcatctggtatggactttcaggaccctggtctccctcgggtctaagaatcaccctccaaccagtggttcatttttctaggagtcccagtcagccccatgaatccacagacttcagcgaatggaagccctccctgagccgtgtttctggcttcaaccaagtcattgctgccttcctctcccctgtctctacacacaccctcagtggggtctgtgaggtctcatgctgggggcagggatctgtggtgaggggtgcttggcagtccttgttgccactctcaagcttcccaagccattcttcacccctttccaagcaagcttcaagcatccaggctgagcagcacggctcggtttggctctctgtcgcatcaggccctgccgctgttggggggcgctaccagcactccctcttttgcacaaactgatgatataaaaggccagtcctaggcacctaggaaaagtctagtgaactctccctgctagatcagcggtcattatgaccctgttgatttctgtgtcagtaactaagcacgcagcaggggagactttgcggtgggcagttcttcgtaccaagcccccccccccaaggatgctgtggcttgttttcagatccatctcattttcctaaaggtgaattctcatgcatggctgagagacatgtgtatgcagctctgcttctgtctcttaatgtcgcttaaggcccttatagggaactggtatatctacttgctccttaaagcaattttctttctgatgccaataggatgggggttaagacaggcagagctttatcactaacaatatagacagccactcttccttctgcctgcgtcttaaaaaataagctgtcccaaggacacaaatgtatattatccatacatgcacgcatatgctcacacacaaactcagactctgaagtctggtagcctgtgaatgttccttttgtaaaatgcttccaaaagcctctttgctccaaccctgtcctaaccatcagaaaccccaaagaaacggttaaggactccccctcagactctccctacccaagcccctacaccaggacctggccagtccttttaagacagactgggaggacacacaggagtcagcctgccccttctgagggcattttcgtgttgtgcagtgatgttcttccttggatgctggcctggaccagccaacgagaccctgcagtctatcccgccctgcctgtttgcttcctgtgcggtggtatcaatatgtaacagtgcctgtttctgctgatttcatgacatgttctggtttgtttctgatgttcgccgtgagcgttcttgtgccgtgttgatgcctttcgtagcattagactttgcacttttaaaaaaaaaaaaaacaaaaatgttgaagcatcgaggaagcatcgaacccagagtggaatgcatggtatggtagctggcctgggacagagggaccctttctcatcttcctttgagttctttgactatctgcttttccagcctctcccgtcttttgtatctggttcaaattattataaaggaaagaccctctgagtataccggttctgaaagacggcctttctgttttccactcatgctggggtttctagccacaccaggcagatgagaggaaaccgagcgagcaaacgaacctttgggacaaagtgccagatggcagctgagcaacagccactcaaatgccttcccagcaaaccaattgcaatatatagtttaaggtgttgttttacttctgttatattctaagccctgggcctccctcccttactcccgtcatgccagcaactcgcaatatttcagatgacgtttacatggtagcaatttccaaatcgctgcctgatgcgtattaagacatatccgtgggcttgctgcataactcaacgctttgttgattttgtttctgtttgaactcttggttgtagggggggaggggtggaaccccatgtgcgtgctggaatatgaagtctgagatgtaccccccaacaccccacgctggcgatacgtgagagttgttgaaagtcagcaagccgagcgcgcctgatgctctgtatcagtctctacttttatttttatgagtttgctctgtcaatggacaataaaccatattatcaaagagaaaaaaaaaaaaaaa

SEQ ID NO: 148 - protein, 220 aa, Mus musculus
MGAAARSLRLALGLLLLATLLRPADACSCSPVHPQQAFCNADVVIRAKAVSEKEVDSGNDIYGNPIKRIQYEIKQIKMFKGPDKDIEFIYTAPSSAVCGVSLDVGGKKEYLIAGKAEGDGKMHITLCDFIVPWDTLSITQKKSLNHRYQMGCECKITRCPMIPCYISSPDECLWMDWVTEKSINGHQAKFFACIKRSDGSCAWYRGAAPPKQEFLDIEDP

SEQ ID NO: 149 - DNA, 1270 bp, Mus musculus
tcccctctccctgctgtccctgctgtcgctgcgcgggagcctagagaggactaggcccggagcatcatggtccacgaggctccgcacgcttcatccttccagatgctgctacagctggcgcttctgctcctgctccgggccgagccactgcggagcgctgagctcaccttcgagctgccggacaacgccaagcagtgcttccacgaagaggtggagcagggcgtgaagttctctctggattaccaggttatcactggaggccactacgatgtggactgctatgtggaagaccccaggggcaacgtcatctacagggagaccaagaagcagtatgacagcttcacgtacaagacagaggccaagggcgtctaccggttttgcttcagtaatgagttctccaccttctctcataagaccgtctactttgacttccaagtgggtgacgagccccccattctcccagacatggggaacagggtcacggctctcactcagatggaatctgcctgtgtaactattcacgaggctctgaagactgtgatcgactcccagacacactatcgactgcgggaggctcaggaccgagcccgagctgaagaccttaatagtcgagtgtcctactggtctgtaggcgagactattgccctgtttgtggtcagcttcagccaggtgctactgctgaaaagcttctttacagaaaagcgacctgttaacagagcggtccactcctagcctcgaagtccctcccacagtccctcacccctcccgggtctcaggttcctacagggctggtggtgtgacacagagaagagggagagcagcagggagaccaaaggcgcacatatctgatctttgcactccaacaacggaagctcacagagccccggtaatctgattgtattgtgtgtgtaacattggccaaaagtgtttacaggtcaacctagggtttggtcactacgggaggccagtgatctagcatccaatctgtaggagaactccaagtgcctggacctccagggcagtttagggttctgccactactccatgagacattggtcattaggtggttgtgtcttcttccatcttgagaaagggaccctgagttcttgccttcccttcgtggacctctctattcctgaggaaagctggtctaatctggaaacagtagggtcagacatacaaccaggacccaagtagagagtgagtgagtgcccttggtgatctatgtctcctggaggcagaactgtgctaataaaaaccaagactgagc

SEQ ID NO: 150 - protein, 221 aa, Mus musculus
MVHEAPHASSFQMLLQLALLLLLRAEPLRSAELTFELPDNAKQCFHEEVEQGVKFSLDYQVITGGHYDVDCYVEDPRGNVIYRETKKQYDSFTYKTEAKGVYRFCFSNEFSTFSHKTVYFDFQVGDEPPILPDMGNRVTALTQMESACVTIHEALKTVIDSQTHYRLREAQDRARAEDLNSRVSYWSVGETIALFVVSFSQVLLLKSFFTEKRPVNRAVHS

SEQ ID NO: 151 - DNA, 1059 bp, Mus musculus
cccacgcgtccggcggagctgtggagcctcctggtctccagcatcagttcctgggtcctgtccgaaggcacgctcaggagcagccaagcggtagaagccgggtggcatggcagcgagcacggacatagctgggctggaggagagcttccggaagtttgccatccatggcgaccccaaggccagcgggcaagagatgaatggcaagaactgggccaagctgtgcaaggactgtaaggtggccgacggaaaggccgtaacgggcaccgacgtcgacatcgtcttctccaaagtcaaggcgaaatctgctagagtaatcaactatgaggagttcaagaaggccctggaagagctggcaactaagcggttcaaggggaagtccaaggaggaggcctttgatgccatctgccagctgatagcgggcaaggaaccggccaacattggcgtcaccaaagctaaaacgggtggtgctgtggaccggctgacggacaccagtaagtatacgggctcccacaaagaacgctttgatgagagcggcaagggaaagggcatcgctggacggcaggacatcctggacgacagtggctacgtgagtgcctacaaaaacgcaggcacctatgacgccaaggtgaagaagtgacacctgccaaagcccccagggaggctgtcccactggaggcccaggcctggggtccagggtcacatggagcaagaaagcctggctccctccctgctggatctgccaccaagagcttcctgcccagtcccagggccatcccatcaggcctctgacccagactgctgtgtcccttcttcctgtctccctgtcatctgtctgggagtcagtgcctatatcctcaccgccccagcctggtcccaggcatggctgactcttgcctgcttttgcctcatatttaagctgctgctctggccaagtgcctaattcttacccagacctcaataaagataccttttgtaccaggaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 152 - protein, 176 aa, Mus musculus
MAASTDIAGLEESFRKFAIHGDPKASGQEMNGKNWAKLCKDCKVADGKAVTGTDVDIVFSKVKAKSARVINYEEFKKALEELATKRFKGKSKEEAFDAICQLIAGKEPANIGVTKAKTGGAVDRLTDTSKYTGSHKERFDESGKGKGIAGRQDILDDSGYVSAYKNAGTYDAKVKK

SEQ ID NO: 153 - DNA, 870 bp, Mus musculus
ggctgaccaatattgaaaagaatagacctggtttgtgaattatggcctggatttcacttatactctctctcctggctctcagctcaggggccatttcccaggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccattgggtgttcggtggaggaaccaaactgactgtcctaggccagcccaagtcttcgccatcagtcaccctgtttccaccttcctctgaagagctcgagactaacaaggccacactggtgtgtacgatcactgatttctacccaggtgtggtgacagtggactggaaggtagatggtacccctgtcactcagggtatggagacaacccagccttccaaacagagcaacaacaagtacatggctagcagctacctgaccctgacagcaagagcatgggaaaggcatagcagttacagctgccaggtcactcatgaaggtcacactgtggagaagagtttgtcccgtgctgactgttcctaggtcatctaaccttcattttacccacagaggctgagatcagaaacatgcccaagtgtatccttggtgcttttgcctaccatagcccttctctctaccctcaaaatgcacaataaaatgttcattc

SEQ ID NO: 154 - protein, 234 aa, Mus musculus
MAWISLILSLLALSSGAISQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 155 - DNA, 551 bp, Mus musculus
gtgaccaatattgaaaagaatagacctggtttgtgaattatggcctggatttcacttatactctctctcctggctctcagctcaggggccatttcccaggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccatttccacaatgacatgtgtagatggggaagtagaacaagaacactctggtacagtctcataactaccatcttcttaacaggtggctacatctccctagtctgttctcttttactatagagaaatttataaaagctgttgtctcaatcaataaaaagttttatttc

SEQ ID NO: 156 - protein, 154 aa, Mus musculus
MAWISLILSLLALSSGAISQAVVTQESALTTSPGETVTLTCRSSTGAVTTSNYANWVQEKPDHLFTGLIGGTNNRAPGVPARFSGSLIGDKAALTITGAQTEDEAIYFCALWYSNHFHNDMCRWGSRTRTLWYSLITTIFLTGGYISLVCSLLL

SEQ ID NO: 157 - DNA, 660 bp, Mus musculus
atggcctggatttcacttatactctctctcctggctctcagctcaggggccatttcccaggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccatttctattttcggcagtggaaccaaggtcactctcctaggtcagcccaagtccactcccacactcaccatgtttccaccttcccctgaggagctccaggaaaacaaagccacactcgtgtgtctgatttccaatttttccccaagtggtgtgacagtggcctggaaggcaaatggtacacctatcacccagggtgtggacacttcaaatcccaccaaagaggacaacaagtacatggccagcagcttcttacatttgacatcggaccagtggagatctcacaacagttttacctgccaagttacacatgaagg

(19) United States
(12) Patent Application Publication
(10) Pub. No.: US 2010/0099090 A1
(43) Pub. Date: Apr. 22, 2010

(54) BIOMARKERS AND METHODS FOR DETERMINING SENSITIVITY TO CTLA-4 ANTAGONISTS

(75) Inventors: Zenta Tsuchihashi, Skillman, NJ (US); Beihong Hu, Ringoes, NJ (US); Maria Jure-Kunkel, Plainsboro, NJ (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(73) Assignee: Bristol-Mayers Squibb Company, Princeton, NJ (US)

(21) Appl. No.: 12/449,942

(22) PCT Filed: Mar. 4, 2008
(86) PCT No.: PCT/US2008/002863

§ 371 Date: Sep. 3, 2009

Related U.S. Application Data
(60) Provisional application No. 60/892,916, filed on Mar. 5, 2007.
(60) Provisional application No. 60/923,117, filed on Apr. 12, 2007.

(51) Int. Cl. C12Q 1/68

(52) U.S. Cl. 435/6

(57) ABSTRACT

CTLA-4 biomarkers useful in a method for predicting the likelihood that a mammal that will respond therapeutically to a method of treating cancer comprising administering an CTLA-4 antagonist, wherein the method comprises (a) measuring in the mammal the level of at least one biomarker s, (b) exposing a biological sample from the mammal to the CTLA-4 antagonist, and (c) following the exposing of step (b), measuring in the biological sample the level of the at least one biomarker, wherein an increase in the level of the at least one biomarker measured in step (c) compared to the level of the at least one biomarker measured in step (a) indicates an increased likelihood that the mammal will respond therapeutically to the method of treating cancer.

This application claims the benefit of U.S. Provisional Application No. 60/892,916, filed Mar. 5, 2007, and U.S. Provisional Application No. 60/923,117, filed Apr. 12, 2007, whose contents are hereby incorporated by reference.

### FIELD OF THE INVENTION

The present invention relates generally to the field of pharmacogenomics. More specifically, the invention relates to methods and procedures to determine drug sensitivity and insensitivity in patients, which allows the identification of individualized genetic profiles which will aid in treating diseases and disorders including cancer. The invention also relates to methods for predicting the likelihood a mammal will respond therapeutically to a method of treating cancer.

### BACKGROUND OF THE INVENTION

Cancer is a disease with extensive histoclinical heterogeneity. Although conventional histological and clinical features have been correlated to prognosis, the same apparent prognostic type of tumors varies widely in its responsiveness to therapy and consequent survival of the patient.

New prognostic and predictive markers, which would facilitate an individualization of therapy for each patient, are needed to accurately predict patient response to treatments, such as small molecule or biological molecule drugs, in the clinic. The problem may be solved by the identification of new parameters that could better predict the patient's sensitivity to treatment. The classification of patient samples is a crucial aspect of cancer diagnosis and treatment. The association of a patient's response to a treatment with molecular and genetic markers can open up new opportunities for treatment development in non-responding patients, or distinguish a treatment's indication among other treatment choices because of higher confidence in the efficacy. Further, the pre-selection of patients who are likely to respond well to a medicine, drug, or combination therapy may reduce the number of patients needed in a clinical study or accelerate the time needed to complete a clinical development program (Cockett et al., Current Opinion in Biotechnology, 11:602-609 (2000)).

The ability to predict drug sensitivity in patients is particularly challenging because drug responses reflect not only properties intrinsic to the target cells, but also a host's metabolic properties. Efforts to use genetic information to predict drug sensitivity have primarily focused on individual genes that have broad effects, such as the multidrug resistance genes, mdr1 and mrp1 (Sonneveld, J. Intern. Med., 247:521-534 (2000)).

The development of microarray technologies for large scale characterization of gene mRNA expression pattern has made it possible to systematically search for molecular markers and to categorize cancers into distinct subgroups not evident by traditional histopathological methods (Khan et al., Cancer Res., 58:5009-5013 (1998); Alizadeh et al., Nature, 403:503-511 (2000); Bittner et al., Nature, 406:536-540 (2000); Khan et al., Nature Medicine, 7(6):673-679 (2001); and Golub et al., Science, 286:531-537 (1999); Alon et al., P. N. A. S. USA, 96:6745-6750 (1999)). Such technologies and molecular tools have made it possible to monitor the expression level of a large number of transcripts within a cell population at any given time (see, e.g., Schena et al., Science, 270:467-470 (1995); Lockhart et al., Nature Biotechnology, 14:1675-1680 (1996); Blanchard et al., Nature Biotechnology, 14:1649 (1996); U.S. Pat. No. 5,569,588).

Recent studies demonstrate that gene expression information generated by microarray analysis of human tumors can predict clinical outcome (van't Veer et al., Nature, 415:530-536 (2002); Sorlie et al., P. N. A. S. USA, 98:10869-10874 (2001); M. Shipp et al., Nature Medicine, 8(1):68-74 (2002): Glinsky et al., The Journal of Clin. Invest., 113(6):913-923 (2004)). These findings bring hope that cancer treatment will be vastly improved by better predicting the response of individual tumors to therapy.

The vertebrate immune system requires multiple signals to achieve optimal immune activation (see, e.g., Janeway, Cold Spring Harbor Symp. Quant. Biol. 1989; 54:1-14; Paul William E., ed. Raven Press, N.Y., Fundamental Immunology, 4th edition (1998), particularly chapters 12 and 13, pages 411 to 478). Interactions between T lymphocytes (T cells) and antigen presenting cells (APC) are essential to the immune response. Levels of many cohesive molecules found on T cells and APC's increase during an immune response (Springer et al., A. Rev. Immunol. 1987; 5:223-252; Shaw and Shimuzu, Current Opinion in Immunology, 1988 Eds. Kindt and Long, 1:92-97; and Hemler, Immunology Today 1988; 9:109-113). Increased levels of these molecules may help explain why activated APC's are more effective at stimulating antigen-specific T cell proliferation than are resting APC's (Kaiuchi et al., J. Immunol. 1983; 131:109-114; Kreiger et al., J. Immunol. 1985; 135:2937-2945; McKenzie, J. Immunol. 1988; 141:2907-2911; and Hawrylowicz and Unanue, J. Immunol. 1988; 141:4083-4088).

T cell immune response is a complex process that involves cell-cell interactions (Springer et al., A. Rev. Immunol. 1987; 5:223-252), particularly between T and accessory cells such as APC's, and production of soluble immune mediators (cytokines or lymphokines) (Dinarello, New Engl. J. Med 1987; 317:940-945; Sallusto, J. Exp. Med. 1997; 179:1109-1118). This response is regulated by several T-cell surface receptors, including the T-cell receptor complex (Weiss, Aim. Rev. Immunol. 1986; 4:593-619) and other “accessory” surface molecules (Allison, Curr. Opin. Immunol. 1994; 6:414-419; Springer, 1987, supra). Many of these accessory molecules are naturally occurring cell surface differentiation (CD) antigens defined by the reactivity of monoclonal antibodies on the surface of cells (McMichael, Ed., Leukocyte Typing III, Oxford Univ. Press, Oxford, N.Y., 1987).

CD28 antigen, a homodimeric glycoprotein of the immunoglobulin superfamily (Aruffo and Seed, Proc. Natl. Acad. Sci. 1987; 84:8573-8577), is an accessory molecule found on most mature human T cells (Damle et al., J. Immunol. 1983; 131:2296-2300). Current evidence suggests that this molecule functions in an alternative T cell activation pathway distinct from that initiated by the T-cell receptor complex (June et al., Mol. Cell. Biol. 1987; 7:4472-4481). Monoclonal antibodies (MAbs) reactive with CD28 antigen can augment T cell responses initiated by various polyclonal stimuli (reviewed by June et al., supra). These stimulatory effects may result from MAb-induced cytokine production (Thompson et al., Proc. Natl. Acad. Sci. 1989; 86:1333-1337; and Lindsten et al., Science 1989; 244:339-343) as a consequence of increased mRNA stabilization (Lindsten et al., 1989, supra).

CTLA-4 is a negative regulator of CD28′ dependent T cell activation, and acts as an inhibitory checkpoint for the adaptive immune response. Various preclinical studies have shown that CTLA-4 blockade by monoclonal antibodies enhances the host immune response against immunogenic tumors, and can even reject established tumors. Currently, ipilimumab (MDX-010) and CP-675206, both fully human anti-human CTLA-4 monoclonal antibodies (mAbs), are under clinical development to treat various types of solid tumors.

CTLA-4 (cytotoxic T lymphocycte-associated antigen-4) is accepted as opposing CD28 activity and dampening T cell activation (Krummel, J. Exp. Med. 1995; 182:459-465; Krummel et al., Int'l Immunol. 1996; 8:519-523; Chambers et al., Immunity. 1997; 7:885-895). CTLA-4 deficient mice suffer from massive lymphoproliferation (Chambers et al., supra). It has been reported that CTLA-4 blockade augments T cell responses in vitro (Walunas et al., Immunity. 1994; 1:405-413) and in vivo (Kearney, J. Immunol. 1995; 155:1032-1036), exacerbates antitumor immunity (Leach, Science 1996; 271:1734-1736), and enhances an induced autoimmune disease (Luhder, J. Exp. Med. 1998; 187:427-432). It has also been reported that CTLA-4 has an alternative or additional impact on the initial character of the T cell immune response (Chambers, Curr. Opin. Immunol. 1997; 9:396-404; Bluestone, J. Immunol. 1997; 158:1989-1993; Thompson, Immunity 1997; 7:445-450). This is consistent with the observation that some autoimmune patients have autoantibodies to CTLA-4. It is possible that CTLA-4 blocking autoantibodies play a pathogenic role in these patients (Matsui, J. Immunol. 1999; 162:4328-4335).

Non-human CTLA-4 antibodies have been used in the various studies discussed above. Furthermore, human antibodies against human CTLA-4 have been described as immunostimulation modulators in a number of disease conditions, such as treating or preventing viral and bacterial infection and for treating cancer (e.g., PCT Publication WO 01/14424 and PCT Publication WO 00/37504). U.S. Pat. No. 5,855,887 discloses a method of increasing the response of a mammalian T cell to antigenic stimulation by combining a T cell with a CTLA-4 blocking agent. U.S. Pat. No. 5,811,097 discloses a method of decreasing the growth of non-T cell tumors by administering a CTLA-4 blocking agent. U.S. Pat. No. 6,984,720 and U.S. Patent Publication No. 2002/0086014 disclose human CTLA-4 antibodies. Each of these patents and applications is hereby incorporated by reference.

Needed are new and alternative methods and procedures to determine drug sensitivity in patients to allow the development of individualized genetic profiles which are necessary to treat diseases and disorders based on patient response at a molecular level.

### SUMMARY OF THE INVENTION

The invention provides methods and procedures for determining patient sensitivity to one or more CTLA-4 antagonists. The invention also provides methods of determining or predicting whether an individual requiring therapy for a disease state such as cancer will or will not respond to treatment, prior to administration of the treatment, wherein the treatment comprises administration of one or more CTLA-4 antagonists. The one or more CTLA-4 antagonists are compounds that can be selected from, for example, one or more small molecule CTLA-4 inhibitors or one or more CTLA-4 binding monoclonal antibodies.

In one aspect, the invention provides a method for predicting the likelihood a mammal will respond therapeutically to a method of treating cancer comprising administering an CTLA-4 antagonist, wherein the method comprises: (a) measuring in the mammal the level of at least one biomarker; (b) exposing a biological sample from the mammal to the CTLA-4 antagonist; (c) following the exposing of step (b), measuring in the biological sample the level of the at least one biomarker, wherein an increase in the level of the at least one biomarker measured in step (c) compared to the level of the at least one biomarker measured in step (a) indicates an increased likelihood that the mammal will respond therapeutically to the method of treating cancer.

In another aspect, the invention provides a method for predicting the likelihood a mammal will respond therapeutically to a method of treating cancer comprising administering an CTLA-4 antagonist, wherein the method comprises: (a) measuring in the mammal the level of at least one biomarker selected from the biomarkers of Table 1 and Table 3; (b) exposing a biological sample from said mammal to the CTLA-4 antagonist; (c) following the exposing of step (b), measuring in said biological sample the level of the at least one biomarker, wherein an increase in the level of the at least one biomarker measured in step (c) compared to the level of the at least one biomarker measured in step (a), indicates an increased likelihood that the mammal will respond therapeutically to said method of treating cancer when said at least one biomarker is from Table 1, and indicates an increased likelihood that the mammal will not respond therapeutically to said method of treating cancer when said at least one biomarker is from Table 3.

The biological sample can comprise, for example, at least one of whole fresh blood, peripheral blood mononuclear cells, frozen whole blood, fresh plasma, frozen plasma, urine, saliva, skin, hair follicle, bone marrow, or tumor tissue. In one aspect, the biological sample is tumor tissue. In another aspect, the biological sample can be, for example, a tissue sample comprising cancer cells and the tissue is fixed, paraffin-embedded, fresh, or frozen.

A difference in the level of the biomarker that is sufficient to predict the likelihood that the mammal will or will not respond therapeutically to the method of treating cancer can be readily determined by one of skill in the art using known techniques. The increase or decrease in the level of the biomarker can be correlated to determine whether the difference is sufficient to predict the likelihood that a mammal will respond therapeutically. The difference in the level of the biomarker that is sufficient can, in one aspect, be predetermined prior to predicting the likelihood that the mammal will respond therapeutically to the treatment. In one aspect, the difference in the level of the biomarker is a difference in the mRNA level (measured, for example, by RT-PCR or a microarray), such as at least a two-fold difference, at least a three-fold difference, or at least a four-fold difference in the level of expression. In another aspect, the difference in the level of the biomarker is determined by IHC. In another aspect, the difference in the level of the biomarker refers to a p-value of <0.05 in Anova (t test) analysis. In yet another aspect, the difference is determined in an ELISA assay.

As used herein, respond therapeutically refers to the alleviation or abrogation of the cancer. This means that the life expectancy of an individual affected with the cancer will be increased or that one or more of the symptoms of the cancer will be reduced or ameliorated. The term encompasses a reduction in cancerous cell growth or tumor volume. Whether a mammal responds therapeutically can be measured by many methods well known in the art, such as PET imaging.

The mammal can be, for example, a human, rat, mouse, dog, rabbit, pig sheep, cow, horse, cat, primate, or monkey.

The method of the invention can be, for example, an in vitro method wherein the step of measuring in the mammal the level of at least one biomarker comprises taking a biological sample from the mammal and then measuring the level of the biomarker(s) in the biological sample. In one aspect, the biological sample is tumor tissue. In another aspect, the biological sample can comprise, for example, at least one of serum, whole fresh blood, peripheral blood mononuclear cells, frozen whole blood, fresh plasma, frozen plasma, urine, saliva, skin, hair follicle, bone marrow, or tumor tissue.

The level of the at least one biomarker can be, for example, the level of protein and/or mRNA transcript of the biomarker. The level of the biomarker can be determined, for example, by RT-PCR or another PCR-based method, immunohistochemistry, proteomics techniques, or any other methods known in the art, or their combination.

In another aspect, the invention provides a method for identifying a mammal that will respond therapeutically to a method of treating cancer comprising administering of an CTLA-4 antagonist, wherein the method comprises: (a) measuring in the mammal the level of at least one biomarker; (b) exposing a biological sample from the mammal to the CTLA-4 antagonist; (c) following the exposing in step (b), measuring in said biological sample the level of the at least one biomarker, wherein a difference in the level of the at least one biomarker measured in step (c) compared to the level of the at least one biomarker measured in step (a) indicates that the mammal will respond therapeutically to the said method of treating cancer.

In another aspect, the invention provides a method for identifying a mammal that will respond therapeutically to a method of treating cancer comprising administering an CTLA-4 antagonist, wherein the method comprises: (a) exposing a biological sample from the mammal to the CTLA-4 antagonist; (b) following the exposing of step (a), measuring in said biological sample the level of at least one biomarker, wherein a difference in the level of the at least one biomarker measured in step (b), compared to the level of the at least one biomarker in a mammal that has not been exposed to said CTLA-4 antagonist, indicates that the mammal will respond therapeutically to said method of treating cancer.

In yet another aspect, the invention provides a method for testing or predicting whether a mammal will respond therapeutically to a method of treating cancer comprising administering an CTLA-4 antagonist, wherein the method comprises: (a) measuring in the mammal the level of at least one biomarker; (b) exposing the mammal to the CTLA-4 antagonist; (c) following the exposing of step (b), measuring in the mammal the level of the at least one biomarker, wherein a difference in the level of the at least one biomarker measured in step (c) compared to the level of the at least one biomarker measured in step (a) indicates that the mammal will respond therapeutically to said method of treating cancer.

In another aspect, the invention provides a method for determining whether a compound inhibits CTLA-4 activity in a mammal, comprising: (a) exposing the mammal to the compound; and (b) following the exposing of step (a), measuring in the mammal the level of at least one biomarker, wherein a difference in the level of said biomarker measured in step (b), compared to the level of the biomarker in a mammal that has not been exposed to said compound, indicates that the compound inhibits CTLA-4 activity in the mammal.

In yet another aspect, the invention provides a method for determining whether a mammal has been exposed to a compound that inhibits CTLA-4 activity, comprising (a) exposing the mammal to the compound; and (b) following the exposing of step (a), measuring in the mammal the level of at least one biomarker, wherein a difference in the level of said biomarker measured in step (b), compared to the level of the biomarker in a mammal that has not been exposed to said compound, indicates that the mammal has been exposed to a compound that inhibits CTLA-4 activity.

In another aspect, the invention provides a method for determining whether a mammal is responding to a compound that inhibits CTLA-4 activity, comprising (a) exposing the mammal to the compound; and (b) following the exposing of step (a), measuring in the mammal the level of at least one biomarker, wherein a difference in the level of the at least one biomarker measured in step (b), compared to the level of the at least one biomarker in a mammal that has not been exposed to said compound, indicates that the mammal is responding to the compound that inhibits CTLA-4 activity.

As used herein, “responding” encompasses responding by way of a biological and cellular response, as well as a clinical response (such as improved symptoms, a therapeutic effect, or an adverse event), in a mammal.

The invention also provides an isolated biomarker. The biomarkers of the invention comprise sequences selected from the nucleotide and amino acid sequences, as well as fragments and variants thereof.

The invention also provides a biomarker set comprising two or more biomarkers.

The invention also provides kits for determining or predicting whether a patient would be susceptible to a treatment that comprises one or more CTLA-4 antagonists. The patient may have a cancer or tumor.

In one aspect, the kit comprises a suitable container that comprises one or more specialized microarrays of the invention, one or more CTLA-4 antagonists for use in testing cells from patient tissue specimens or patient samples, and instructions for use. The kit may further comprise reagents or materials for monitoring the expression of a biomarker set at the level of mRNA or protein.

In another aspect, the invention provides a kit comprising two or more biomarkers.

In yet another aspect, the invention provides a kit comprising at least one of an antibody and a nucleic acid for detecting the presence of at least one of the biomarkers. In one aspect, the kit further comprises instructions for determining whether or not a mammal will respond therapeutically to a method of treating cancer comprising administering a compound that inhibits CTLA-4 activity. In another aspect, the instructions comprise the steps of (a) measuring in the mammal the level of at least one biomarker, (b) exposing the mammal to the compound, (c) following the exposing of step (b), measuring in the mammal the level of the at least one biomarker, wherein a difference in the level of the at least one biomarker measured in step (c) compared to the level of the at least one biomarker measured in step (a) indicates that the mammal will respond therapeutically to said method of treating cancer.

The invention also provides screening assays for determining if a patient will be susceptible to treatment with one or more CTLA-4 antagonists.

The invention also provides a method of monitoring the treatment of a patient having a disease, wherein said disease is treated by a method comprising administering one or more CTLA-4 antagonists.

The invention also provides individualized genetic profiles which are necessary to treat diseases and disorders based on patient response at a molecular level.

The invention also provides specialized microarrays, e.g., oligonucleotide microarrays or cDNA microarrays, comprising one or more biomarkers having expression profiles that correlate with sensitivity to one or more CTLA-4 antagonists.

The invention also provides antibodies, including polyclonal or monoclonal, directed against one or more biomarkers of the invention.

The invention will be better understood upon a reading of the detailed description of the invention when considered in connection with any accompanying figures.

### BRIEF DESCRIPTION OF THE FIGURES

FIG. 1 illustrates the results obtained of the anti-tumor activity of UC10 in the Sa1N tumor model.

FIG. 2 illustrates the results obtained from immunohistochemistry staining.

FIG. 3 (FIGS. 3A and 3B) illustrates the results obtained from expansion of T cells with an effector/memory phenotype following UC treatment.

FIG. 4 illustrates the results obtained from qPCR analysis of tumor RNA

FIG. 5 illustrates the results obtained from qPCR analysis of peripheral blood RNA.

FIG. 6 illustrates the results obtained from induction of T cell receptor, immunoglobulin, and class II MHC genes.

FIG. 7 illustrates the results obtained showing a lack of anti-tumor activity of UC10 in the EMT6 tumor model.

FIG. 8 illustrates the results obtained from qPCR analysis of tumor RNA.

FIG. 9 illustrates the results obtained from qPCR analysis of peripheral blood RNA.

FIG. 10 illustrates the results obtained from measuring the time course of Ym1 and arginase 1 gene expression in the blood.

### DETAILED DESCRIPTION OF THE INVENTION

As used herein, the terms “cytotoxic T lymphocyte-associated antigen-4,” “CTLA-4,” “CTLA4,” “CTLA-4 antigen” and “CD152” (see, e.g., Murata, Am. J. Pathol. 1999; 155:453-460) are used interchangeably, and include variants, isoforms, species homologs of human CTLA-4, and analogs having at least one common epitope with CTLA-4 (see, e.g., Balzano (1992) Int. J. Cancer Suppl. 7:28-32). CTLA-4's complete sequence is found in GenBank Accession No. L15006.

The human monoclonal antibody MDX-010 (Medarex, Inc.) in clinical development corresponds to monoclonal antibody 10D1, which is disclosed in U.S. Patent Publication No. 20050201994 and PCT Publication No. WO 01/14424. MDX-010 has been administered as single or multiple doses, alone or in combination with a vaccine, chemotherapy, or interleukin-2 to greater than 500 patients diagnosed with metastatic melanoma, prostate cancer, lymphoma, renal cell cancer, breast cancer, ovarian cancer, and HIV.

Other anti-CTLA-4 antibodies that can be used in a method of the present invention include, for example, those disclosed in: WO 98/42752; WO 00/37504; U.S. Pat. No. 6,207,156; Hurwitz et al., PNAS 1998; 95(17):10067-10071; Camacho et al., J Clin Oncology 2004:22(145):abstract no. 2505 (antibody CP-675206); and Mokyr, et al., Cancer Research 1998; 58:5301-5304.

In this study, the effect of anti-CTLA-4 treatment on various biomarkers was characterized in mouse tumor models with the goal to obtain candidate biomarkers useful in monitoring the biological effects of this treatment in the clinical setting. Two mouse tumor models, Sa1N (fibrosarcoma, ALT mouse strain) and EMT-6 (mammary carcinoma, Balb/c mouse strain), were used in these studies. The anti-CTLA-4 treatment was highly efficacious against Sa1N tumors, producing complete regressions in most of the treated mice. However, it showed no inhibitory effect on the growth of EMT-6 tumors. Tumor and blood samples from SA1N and EMT-6-bearing mice treated with anti-CTLA-4 antibody or control vehicle were collected at various timepoints. Several selected markers were analyzed by immunohistochemistry (IHC) (tumor only) or RT-PCR (tumor and blood). A significant increase of CD4+ and CD8+ T cells were detected in SA1N tumors (sensitive model) upon treatment but not in EMT-6 tumors (resistant model). Gene expression analysis corroborated the IHC result by showing the induction of various immune response related genes including Cd3d, Cd4, Cd8b1, interferon-γ, perforin 1, and granzyme B in Sa1N tumor, but not in EMT6. These gene expression changes in the tumor tissue reflected the anti-tumor immune reaction induced by this treatment. In the peripheral blood, anti-CTLA-4 treatment in Sa1N-bearing mice also induced IFN-γ, perforin 1, and granzyme B genes, indicating the systemic elevation of Th1 and cytotoxic immune responses. However the same treatment failed to raise the levels of these genes in the EMT6 model blood, and instead it induced the genes related to the alternative activation of macrophages, such as Chi313 (Ym1) and Retnla (Fizz1). Such changes of the gene expression patterns in the peripheral blood reflect different types of immune response induced by this treatment, and may provide useful biomarkers to monitor the action of anti-CTLA4 treatment in the clinical setting.

The invention provides biomarkers that correlate with CTLA-4 antagonist sensitivity or resistance. These biomarkers can be employed for predicting response to one or more CTLA-4 antagonists. The biomarkers of the invention include polynucleotide and polypeptide sequences.

The biomarkers have expression levels in cells that may be dependent on the activity of the CTLA-4 signal transduction pathway, and that are also highly correlated with CTLA-4 antagonist sensitivity exhibited by the cells. Biomarkers serve as useful molecular tools for predicting the likelihood of a response to CTLA-4 antagonists, preferably biological molecules, small molecules, and the like that affect CTLA-4 kinase activity via direct or indirect inhibition or antagonism of CTLA-4 kinase function or activity.

### Biomarkers and Biomarker Sets

The invention includes individual biomarkers and biomarker sets having both diagnostic and prognostic value in disease areas in which signaling through CTLA-4 or the CTLA-4 pathway is of importance, e.g., in cancers or tumors, in immunological disorders, conditions or dysfunctions, or in disease states in which cell signaling and/or cellular proliferation controls are abnormal or aberrant. The biomarker sets comprise a plurality of biomarkers that highly correlate with sensitivity to one or more CTLA-4 antagonists.

The biomarkers and biomarker sets of the invention enable one to predict or reasonably foretell the likely effect of one or more CTLA-4 antagonists in different biological systems or for cellular responses. The biomarkers and biomarker sets can be used in in vitro assays of CTLA-4 antagonist response by test cells to predict in vivo outcome. In accordance with the invention, the various biomarkers and biomarker sets described herein, or the combination of these biomarker sets with other biomarkers or markers, can be used, for example, to predict how patients with cancer might respond to therapeutic intervention with one or more CTLA-4 antagonists.

A biomarker and biomarker set of cellular gene expression patterns correlating with sensitivity of cells following exposure of the cells to one or more CTLA-4 antagonists provides a useful tool for screening one or more tumor samples before treatment with the CTLA-4 antagonist. The screening allows a prediction of cells of a tumor sample exposed to one or more CTLA-4 antagonists, based on the expression results of the biomarker and biomarker set, as to whether or not the tumor, and hence a patient harboring the tumor, will or will not respond to treatment with the CTLA-4 antagonist.

The biomarker or biomarker set can also be used as described herein for monitoring the progress of disease treatment or therapy in those patients undergoing treatment for a disease involving an CTLA-4 antagonist.

The biomarkers also serve as targets for the development of therapies for disease treatment. Such targets may be particularly applicable to treatment of colorectal cancer. Indeed, because these biomarkers are differentially expressed in sensitive cells, their expression patterns are correlated with relative intrinsic sensitivity of cells to treatment with CTLA-4 antagonists.

The level of biomarker protein and/or mRNA can be determined using methods well known to those skilled in the art. For example, quantification of protein can be carried out using methods such as ELISA, 2-dimensional SDS PAGE, Western blot, immunopreciptation, immunohistochemistry, fluorescence activated cell sorting (FACS), or flow cytometry. Quantification of mRNA can be carried out using methods such as PCR, array hybridization, Northern blot, in-situ hybridization, dot-blot, Taqman, or RNAse protection assay.

### Microarrays

The invention also includes specialized microarrays, e.g., oligonucleotide microarrays or cDNA microarrays, comprising one or more biomarkers, showing expression profiles that correlate with sensitivity to one or more CTLA-4 antagonists. Such microarrays can be employed in in vitro assays for assessing the expression level of the biomarkers in the test cells from tumor biopsies, and determining whether these test cells are likely to be sensitive to CTLA-4 antagonists. For example, a specialized microarray can be prepared using all the biomarkers, or subsets thereof, as described herein. Cells from a tissue or organ biopsy can be isolated and exposed to one or more of the CTLA-4 antagonists. In one aspect, following application of nucleic acids isolated from both untreated and treated cells to one or more of the specialized microarrays, the pattern of gene expression of the tested cells can be determined and compared with that of the biomarker pattern from the control panel of cells used to create the biomarker set on the microarray. Based upon the gene expression pattern results from the cells that underwent testing, it can be determined if the cells show a sensitive profile of gene expression. Whether or not the tested cells from a tissue or organ biopsy will respond to one or more of the CTLA-4 antagonists and the course of treatment or therapy can then be determined or evaluated based on the information gleaned from the results of the specialized microarray analysis.

### Antibodies

The invention also includes antibodies, including polyclonal or monoclonal, directed against one or more of the polypeptide biomarkers. Such antibodies can be used in a variety of ways, for example, to purify, detect, and target the biomarkers of the invention, including both in vitro and in vivo diagnostic, detection, screening, and/or therapeutic methods.

### Kits

The invention also includes kits for determining or predicting whether a patient would be susceptible to a treatment that comprises one or more CTLA-4 antagonists. The patient may have a cancer or tumor such as, for example, colorectal cancer. Such kits would be useful in a clinical setting for use in testing a patient's biopsied tumor or other cancer samples, for example, to determine or predict if the patient's tumor or cancer will be sensitive to a given treatment or therapy with an CTLA-4 antagonist. The kit comprises a suitable container that comprises: one or more microarrays, e.g., oligonucleotide microarrays or cDNA microarrays, that comprise those biomarkers that correlate with sensitivity to CTLA-4 antagonists, particularly CTLA-4 inhibitors; one or more CTLA-4 antagonists for use in testing cells from patient tissue specimens or patient samples; and instructions for use. In addition, kits contemplated by the invention can further include, for example, reagents or materials for monitoring the expression of biomarkers of the invention at the level of mRNA or protein, using other techniques and systems practiced in the art such as, for example, RT-PCR assays, which employ primers designed on the basis of one or more of the biomarkers described herein, immunoassays, such as enzyme linked immunosorbent assays (ELISAs), immunoblotting, e.g., Western blots, or in situ hybridization, and the like.

### Application of Biomarkers and Biomarker Sets

The biomarkers and biomarker sets may be used in different applications.

Biomarker sets can be built from any combination of biomarkers to make predictions about the effect of an CTLA-4 antagonist in different biological systems. The various biomarkers and biomarkers sets described herein can be used, for example, as diagnostic or prognostic indicators in disease management, to predict how patients with cancer might respond to therapeutic intervention with compounds that modulate the CTLA-4, and to predict how patients might respond to therapeutic intervention that modulates signaling through the entire CTLA-4 regulatory pathway.

The biomarkers have both diagnostic and prognostic value in diseases areas in which signaling through CTLA-4 or the CTLA-4 pathway is of importance, e.g., in immunology, or in cancers or tumors in which cell signaling and/or proliferation controls have gone awry.

In one aspect, cells from a patient tissue sample, e.g., a tumor or cancer biopsy, can be assayed to determine the expression pattern of one or more biomarkers prior to treatment with one or more CTLA-4 antagonists. In one aspect, the tumor or cancer is colorectal. Success or failure of a treatment can be determined based on the biomarker expression pattern of the cells from the test tissue (test cells), e.g., tumor or cancer biopsy, as being relatively similar or different from the expression pattern of a control set of the one or more biomarkers. Thus, if the test cells show a biomarker expression profile which corresponds to that of the biomarkers in the control panel of cells which are sensitive to the CTLA-4 antagonist, it is highly likely or predicted that the individual's cancer or tumor will respond favorably to treatment with the CTLA-4 antagonist.

The invention also provides a method of monitoring the treatment of a patient having a disease treatable by one or more CTLA-4 antagonists. The isolated test cells from the patient's tissue sample, e.g., a tumor biopsy or tumor sample, can be assayed to determine the expression pattern of one or more biomarkers before and after exposure to an CTLA-4 antagonist wherein, preferably, the CTLA-4 antagonist is an CTLA-4 inhibitor. The resulting biomarker expression profile of the test cells before and after treatment is compared with that of one or more biomarkers as described and shown herein to be highly expressed in the control panel of cells that are sensitive to an CTLA-4 antagonist. Thus, if a patient's response is sensitive to treatment by an CTLA-4 antagonist, based on correlation of the expression profile of the one or biomarkers, the patient's treatment prognosis can be qualified as favorable and treatment can continue. Also, if, after treatment with an CTLA-4 antagonist, the test cells don't show a change in the biomarker expression profile corresponding to the control panel of cells that are sensitive to the CTLA-4 antagonist, it can serve as an indicator that the current treatment should be modified, changed, or even discontinued. This monitoring process can indicate success or failure of a patient's treatment with an CTLA-4 antagonist and such monitoring processes can be repeated as necessary or desired.

### EXAMPLES

In Examples 1 and 2, the effect of an anti-CTLA4 antibody (UC10) was investigated on the biomarker patterns in a sensitive mouse tumor model (Sa1N fibrosarcoma) and a resistant model (EMT6 mammary carcinoma). UC10 is known in the art and is described, for example, in T. Walunas et al., Immunity., August; 1(5):405-13 (1994).

### RNA Isolation:

In the Sa1N model, UC10 induced the infiltration of T cells into the tumor. The gene expression analysis supported this by detecting the induction of various immune response genes including interferon gamma, granzyme B and perforin 1. Gene expression data also indicated the activation of humoral immunity and antigen presentation in the tumor. The activation of interferon gamma, granzyme B and perforin 1 genes was also observed in the peripheral blood. These result indicated the checkpoint blockade by the anti-CTLA4 antibody induced a wide range of immune responses, including those that are crucial to the anti-tumor response.

On the other hand, the effect of UC10 on the EMT6 (resistant model) was entirely different. There, we observed no increase of tumor infiltrating lymphocytes, and no induction of interferon gamma, granzyme B and perforin 1 in the tumor. Thus, a biomarker signal of anti-tumor immunity was not detected in this model. Thus, it appears UC10 failed to modulate any immune response to EMT6 tumors.

However, in the peripheral blood of mice bearing EMT6 tumors, UC10 induced the genes that were the hallmark of alternatively activated macrophages (M2), such as Ym1, Fizz1, and arginase 1.

Currently, it is not known what is the exact type of cell that expresses these genes in the peripheral blood. However, the close resemblance of the induced gene expression pattern with that of alternatively activated macrophages suggests that this type of macrophages or a related type of cells may be highly induced by the UC10 treatment in this model. Alternatively, activated macrophages can be induced by Th2 cytokines and are known to dampen the anti-tumor immune response at least in some cases. Balb/c mice (host of EMT6 tumor) are genetically predisposed to the Th2 response, and the induction of alternatively activated macrophage type genes by CTLA4 blockade with UC10 may be due to this genetic predisposition of the host mice. This may be one of the reasons why this anti-CTLA4 antibody was unable to mount an effective anti-tumor immunity in this model.

In Examples 1 and 2, tumor samples were collected in RNAlater reagent (Ambion, Inc., Austin, Tex.) following the manufacturer's manual. Tumor RNA was extracted using Qiagen RNAeasy mini kit. The quality of isolated RNA was confirmed using Agilent 2100 nucleic acid analyzer detecting the peaks for 18S and 28S rRNA.

### Affymetrix GeneChip Process for Examples 1 and 2:

Mouse GeneChip 430A, v2.0 was used and globin reduction pretreatment was required. Experimental process (cDNA synthesis, in vitro transcription/IVT, and GeneChip hybridization) followed the standard Affymetrix manual. Transcriptional profiling was performed on the RNA obtained from the tumor samples. The Affymetrix GeneChip system (Affymetrix, Santa Clara, Calif.) was used for hybridization and scanning of the mouse 430A arrays. Data were preprocessed using the MAS 5.0 software. Generation of cRNA followed a standard T7 amplification protocol. Total RNA was reverse-transcribed with SuperScript II (Gibco, Carlsbad, Calif.) in the presence of T7-(dT)₂₄ primer to generate first strand cDNA. A second-strand cDNA synthesis was performed in the presence of DNA Polymerase I, DNA ligase, and RNase H (Gibco). The resulting double-stranded cDNA was blunt-ended using T4 DNA polymerase. This double-stranded cDNA was then transcribed into cRNA in the presence of biotin-ribonucleotides using the BioArray High Yield RNA transcript labeling kit (Enzo Life Sciences, Farmingdale, N.Y.). The amplified, biotin-labeled cRNA was purified using Qiagen RNeasy columns (Qiagen Sciences), quantified and fragmented at 94° C. for 35 minutes in the presence of fragmentation buffer (1×). Fragmented cRNA was hybridized to the Affymetrix 430A arrays overnight at 42° C. The arrays were then placed in the fluidics stations for staining and washing as recommended by Affymetrix protocols. The chips were scanned and raw intensity values were generated for each probe on the arrays. The trimmed mean intensity for each array was scaled to 1,500 to account for minor differences in global chip intensity so that the overall expression level for each sample was comparable.

### Data Analysis for Examples 1 and 2:

The GeneChip data was uploaded to PartekPro Pattern Recognition software (Partek, St. Louis, Mo.) for data analysis after using the RMA (Robust Multi-array Analysis) normalization procedure (Irizarry et al., Biostatistics, April; 4(2):249-64 (2003)) with a log 2 transformation.

### Example 1

### Identification of Biomarkers Using Sa1N Mouse Tumor Model

The effect of the anti-CTLA4 monoclonal antibody UC10 was studied on various biomarkers in the Sa1N fibrosarcoma model. (D. Leach et al., J. Immunol., January 15; 154(2):738-43 (1995))

Tumor and peripheral blood samples were collected for RNA analysis. Both the tumor and the blood RNA samples were analyzed by quantitative real-time PCR (qPCR). This was followed up by RNA expression profile analysis of the tumor samples using Affymetrix GeneChip.

### Animal Sample Collection:

SA1N tumor cells were injected in the subcutaneous space of A/J mice. Treatments were initiated on day 6 post implantation when the subcutaneous tumor reached a median size of approximately 145-160 mm³. 0.3125, 1.25, 5, or 20 mg/kg of anti-CTLA4 monoclonal antibody UC10 or 0.2 mL of PBS (phosphate buffered saline) was injected intraperitoneally every three days for three doses (q3dx3). At days 4 (one day post second injection), 7 (one day post third injection), and 11 (seven days post third injection), four mice from each treatment arm were sacrificed.

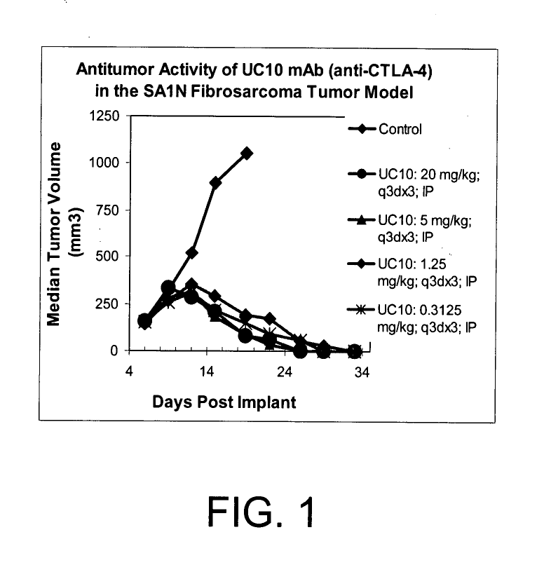

As shown in FIG. 1, UC10 treatments resulted in complete regressions of SA1N tumors in >50% of mice at doses>0.3 mg/kg.

### SA1N Immunohistochemistry Staining:

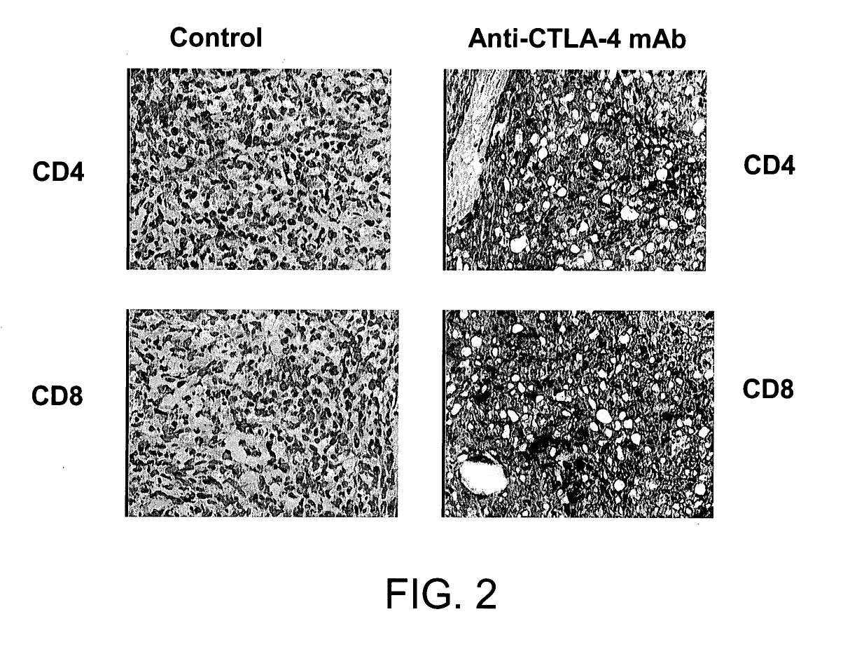

Tumors were collected and frozen 1 day following the third dose of anti-CTLA-4 mAb (5 mg/kg, day 7). Tumor sections were stained with anti-mouse CD4 and anti-mouse CD8 monoclonal antibodies (BD Pharmingen). Higher infiltration of CD4 and CD8 T cells were observed in tumors from animals treated with anti-CTLA-4 mAb vs. control mice (FIG. 2)

Expansion of T Cells with an Effector/Memory Phenotype Following Anti-CTLA-4 Treatment:

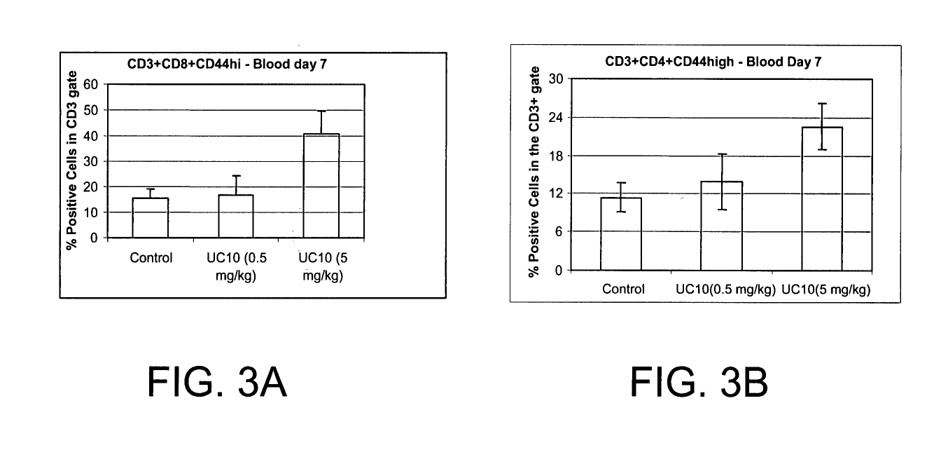

UC10 did not affect frequencies of CD4 or CD8 T cells in blood at the times tested. However, an expansion of CD44high was observed preferentially in the CD8 T cell subset, suggesting an induction of an effector/memory T cell response. (FIG. 3A and FIG. 3B)

qPCR Analysis:

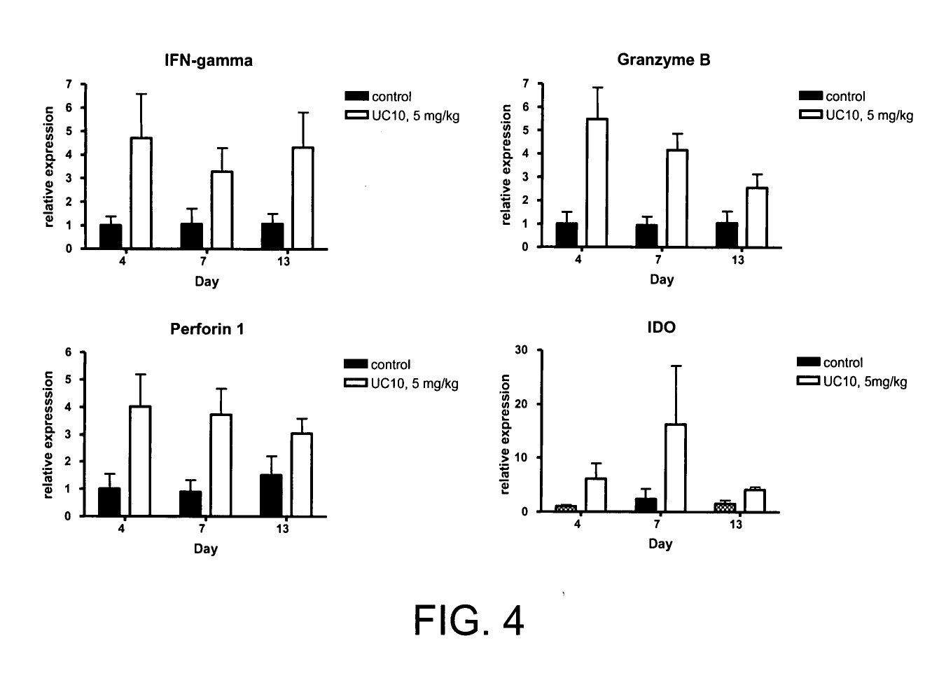

Tumor RNA samples were analyzed by quantitative PCR (qPCR) using Taqman assay on demand reagents (Applied Biosystems) and results are illustrated in FIG. 4. Interferon-gamma and cytotoxic T-cell effector genes (granzyme B and perforin 1) as well as T cell marker genes (Cd3d, Cd4, Cd8b1; not shown) were induced by UC10 in the tumor. The gene for indoleamine-pyrrole 2,3 dioxygenase (IDO) was also induced. IDO is an interferon-gamma inducible gene and can potentially be immunosuppressive.

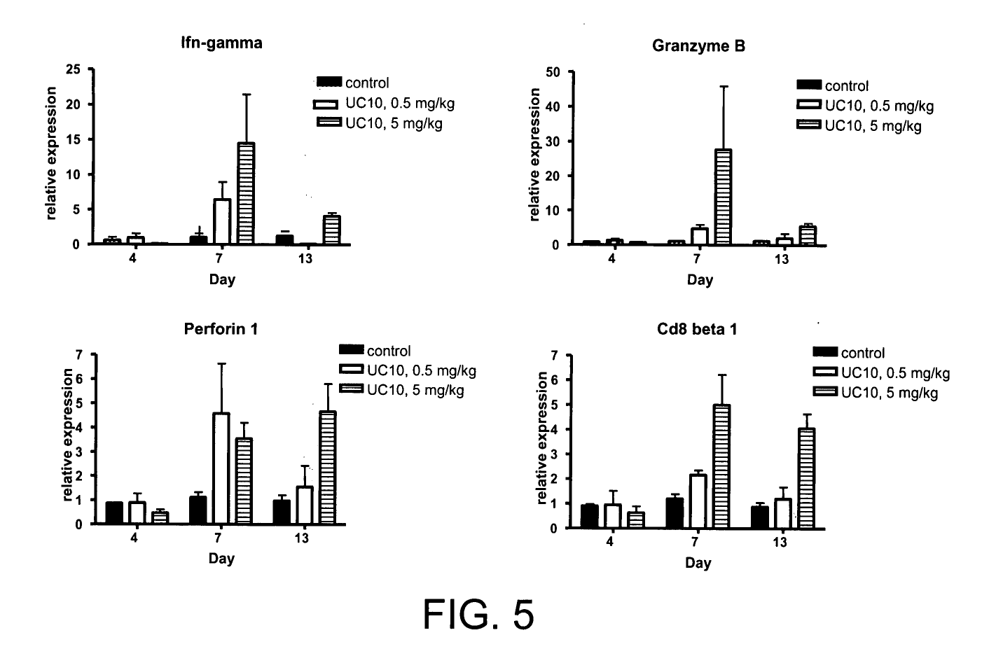

Quantitative PCR (qPCR) analysis of peripheral blood RNA was also performed and the results are illustrated in FIG. 5. Interferon gamma, Cd8b1, granzyme B and perforin 1 genes were all induced in the peripheral blood by UC10. The expression of interferon gamma, granzyme B and Cd8b1 was highest at day 7 (one day after the 3rd injection), and it also was higher at the fully efficacious dose (5 mg/kg) than at the sub-optimal dose (0.5 mg/kg).

### Tumor RNA Expression Analysis:

The tumor cells were collected and tumor RNA extracted as described above. The Affymetrix GeneChip process and data analysis as described above.

### RNA Expression Profiling:

To further identify genes induced by UC10 in the tumor, the same RNA samples were analyzed by RNA expression profiling using Affymetrix mouse A430_{—}2 GeneChip.

The RNA expression data was exported from Xpress using the RMA method with log₂ transformation. The entire data was loaded onto Partek software and the subsequent analysis was performed using this software. Initially, the genes were filtered to select for those with a reliable signal and inter sample variations, applying the following two criteria: (i) CV>10% (to remove the genes that did not have significant variation among different samples); and (ii) the maximal signal >5 (to select genes with a reliable expression level at least in one of the samples).

The genes induced by UC10 were identified using a t-test comparing the expression levels in all the UC10 treated samples (all time points combined) and the control samples (all the time points combined) and are provided in Table 1A.

TABLE 1A
Top 30 genes induced by UC10 (ranked by p-values)

| | | | Fold |
|---|---|---|---|
| Probe Set ID | Gene Symbol | p-value | change |
| 1419762_at | Ubd | 7.56E−07 | 6.18 |
| 1422527_at | H2-DMa | 2.53E−06 | 5.67 |
| 1424923_at | Serpina3g | 4.07E−06 | 6.26 |
| 1418641_at | Lcp2 | 4.20E−06 | 3.05 |
| 1418638_at | — | 7.49E−06 | 2.62 |
| 1449556_at | H2-T23 /// C920025E04Rik | 1.20E−05 | 2.77 |
| 1419004_s_at | Bcl2a1a /// Bcl2a1b /// Bcl2a1d | 1.46E−05 | 2.89 |
| 1450678_at | — | 1.49E−05 | 3.01 |
| 1449580_s_at | H2-DMb1 /// H2-DMb2 | 1.96E−05 | 4.90 |
| 1420915_at | Stat1 | 2.64E−05 | 2.76 |
| 1460218_at | Cd52 | 2.89E−05 | 4.96 |
| 1417025_at | H2-Eb1 | 3.69E−05 | 7.30 |
| 1425477_x_at | H2-Ab1 /// Rmcs1 | 3.77E−05 | 5.26 |
| 1451721_a_at | H2-Ab1 | 4.43E−05 | 5.49 |
| 1419060_at | Gzmb | 4.62E−05 | 4.50 |
| 1452117_a_at | Fyb | 4.84E−05 | 3.34 |
| 1433741_at | Cd38 | 5.22E−05 | 3.34 |
| 1450648_s_at | H2-Ab1 /// Rmcs1 | 5.43E−05 | 5.73 |
| 1448786_at | 1100001H23Rik | 5.65E−05 | 3.10 |
| 1435176_a_at | Idb2 | 6.76E−05 | 2.69 |
| 1422124_a_at | Ptprc | 6.82E−05 | 4.90 |
| 1450753_at | Nkg7 | 6.88E−05 | 3.75 |
| 1425519_a_at | Ii | 8.26E−05 | 5.42 |
| 1454268_a_at | Cyba | 8.64E−05 | 2.52 |
| 1416016_at | — | 8.67E−05 | 2.18 |
| 1431008_at | 0610037M15Rik | 8.68E−05 | 3.08 |
| 1451318_a_at | Lyn | 8.78E−05 | 2.30 |
| 1416296_at | — | 9.83E−05 | 3.35 |
| 1422903_at | Ly86 | 0.0001101 | 3.32 |
| 1423467_at | Ms4a4b | 0.0001104 | 12.83 |

The top 15 genes (probes) of Table 1A included various class II MHC genes as well as other genes important in immune response. Various other genes involved in T cell and B cell activation were also induced. Table 1 provides the biomarkers of Table 1A.

TABLE 1
Biomarkers of Table 1A

| | | | | | | Protein | |
|---|---|---|---|---|---|---|---|
| | | | | DNA | | SEQ | |
| | NCBI Gene | Gene | Probe | SEQ | DNA | ID | Protein |
| Entry | (LocusLink) Title | Symbol | Set ID | ID NO: | Accession | NO: | Accession |
| 1 | Ubiquitin D | Ubd | 1419762_at | 1 | NM_023137.2 | 2 | NP_075626.1 |
| 2 | Histocompatibility | H2- | 1422527_at | 3 | AK146950.1 | 4 | BAE27559.1 |
| | 2, class II, locus | DMa | | | | | |
| | DMa {POOR HIT | (66%) | | | | | |
| | 66%} | | | | | | |
| 3 | Serine (Or cysteine) | Serpina | 1424923_at | 5 | NM_009251.1 | 6 | NP_033277.1 |
| | peptidase inhibitor, | 3g | | | | | |
| | clade A, member | | | | | | |
| | 3G | | | | | | |
| 4 | Lymphocyte | Lcp2 | 1418641_at | 7 | NM_010696.3 | 8 | NP_034826.2 |
| | cytosolic protein 2 | | | | | | |
| 5 | Histocompatibility | H2- | 1418638_at | 9; 11 | AK170866.1; | 10; 12 | BAE42080.1; |
| | 2, class II, locus | DMb1 | | | BC002237.1 | | AAH02237.1 |
| | Mb1 | (??) | | | | | |
| | {UNVALIDATED} | | | | | | |
| 6 | similar to H-2 class | N/A; | 1449556_at | 13; 15; | NM_010398.1; | 14; 16; | NP_034528.1; |
| | I histocompatibility | N/A; | | 17; 19; | XM_904658.2; | 18; 20 | XP_909751.1; |
| | antigen, D-37 alpha | N/A; | | 21 | XM_975970.1; | | XP_981064.1; |
| | chain precursor; | H2-T23 | | | XM_992574.1; | | XP_997668.1 |
| | similar to H-2 class | | | | XR_003960.1 | | |
| | I histocompatibility | | | | | | |
| | antigen, D-37 alpha | | | | | | |
| | chain precursor; | | | | | | |
| | similar to H-2 class | | | | | | |
| | I histocompatibility | | | | | | |
| | antigen, D-37 alpha | | | | | | |
| | chain precursor; | | | | | | |
| | histocompatibility | | | | | | |
| | 2, T region locus 23 | | | | | | |
| 7 | B-cell | Bcl2a1a; | 1419004_s_at | 22; 24; | NM_007534.1; | 23; 25; | NP_031560.1; |
| | leukemia/lymphoma | Bcl2a1b; | | 26 | NM_007536.2; | 27 | NP_031562.1; |
| | 2 related protein | Bcl2a1d | | | NM_009742.3 | | NP_033872.1 |
| | A1a; B-cell | | | | | | |
| | leukemia/lymphoma | | | | | | |
| | 2 related protein | | | | | | |
| | A1b; B-cell | | | | | | |
| | leukemia/lymphoma | | | | | | |
| | 2 related protein | | | | | | |
| | A1d | | | | | | |
| 8 | Integrin beta 2 | Itgb2 | 1450678_at | 28 | NM_008404.2 | 29 | NP_032430.2 |
| 9 | Histocompatibility | H2- | 1449580_s_at | 30; 32 | NM_010387.2; | 31; 33 | NP_034517.2; |
| | 2, class II, locus | DMb1; | | | NM_010388.2 | | NP_034518.1 |
| | Mb1; | H2- | | | | | |
| | histocompatibility | DMb2 | | | | | |
| | 2, class II, locus | | | | | | |
| | Mb2 | | | | | | |
| 10 | Signal transducer | Stat1 | 1420915_at | 34 | NM_009283.3 | 35 | NP_033309.3 |
| | and activator of | | | | | | |
| | transcription 1 | | | | | | |
| 11 | CD52 antigen | Cd52 | 1460218_at | 36 | NM_013706.1 | 37 | NP_038734.1 |
| 12 | N/A; | N/A; | 1417025_at | 38; 39 | AK005018.1; | 40 | BAE33527.1 |
| | histocompatibility | H2-Eb1 | | | AK155968.1 | | |
| | 2, class II antigen E | (74%) | | | | | |
| | beta {POOR HIT | | | | | | |
| | 74%} | | | | | | |
| 13 | Histocompatibility | H2-Ab1 | 1425477_x_at | 41; 43 | M15848.1; | 42; 44 | AAA39547.1; |
| | 2, class II antigen | (72%) | | | NM_207105.1 | | NP_99698.1 |
| | A, beta 1 {POOR | | | | | | |
| | HIT 72%} | | | | | | |
| 14 | N/A; | N/A; | 1451721_a_at | 41; 45; | M15848.1; | 42; 46; | AAA39547.1; |
| | Histocompatibility | H2-Ab1 | | 47; 49; | BC008168.1; | 48; 51; | AAH08168.1; |
| | 2, class II antigen | | | 50; 52 | BC057998.1; | 53 | AAH57998.1; |
| | A, beta 1 | | | | ENSMUST00000040828.4; | | AAA39633.1; |
| | | | | | M13537.1; | | AAA39635.1 |
| | | | | | M13539.1 | | |
| 15 | !! [Gzmb] | | 1419060_at | 54 | ENSMUST00000015581.3 | | |
| | Granzyme B | | | | | | |
| 16 | FYN binding | Fyb | 1452117_a_at | 55 | NM_011815.1 | 56 | NP_035945.1 |
| | protein | | | | | | |
| 17 | CD38 antigen | Cd38 | 1433741_at | 57 | NM_007646.2 | 58 | NP_031672.2 |
| 18 | Histocompatibility | H2-Ab1 | 1450648_s_at | 43 | NM_207105.1 | 44 | NP_996988.1 |
| | 2, class II antigen | | | | | | |
| | A, beta 1 | | | | | | |
| 19 | RIKEN cDNA | 1100001H23Rik | 1448786_at | 59; 61; | NM_025806.1; | 60; 62; | NP_080082.1; |
| | 1100001H23 gene | | | 63 | XM_974622.1; | 64 | XP_979716.1; |
| | | | | | XM_974657.1 | | XP_979751.1 |
| 20 | N/A; Inhibitor of | N/A; | 1435176_a_at | 65; 66; | BF019883.1; | 68 | NP_034626.1 |
| | DNA binding 2 | Id2 | | 67 | ENSMUST00000020974.3; | | |
| | | | | | NM_010496.2 | | |
| 21 | Protein tyrosine | Ptprc | 1422124_a_at | 69 | NM_011210.2 | 70 | NP_035340.2 |
| | phosphatase, | | | | | | |
| | receptor type, C | | | | | | |
| 22 | Natural killer cell | Nkg7 | 1450753_at | 71 | NM_024253.4 | 72 | NP_077215.2 |
| | group 7 sequence | | | | | | |
| 23 | CD74 antigen | Cd74 | 1425519_a_at | 73 | NM_010545.3 | 74 | NP_034675.1 |
| | (invariant | | | | | | |
| | polypeptide of | | | | | | |
| | major | | | | | | |
| | histocompatibility | | | | | | |
| | complex, class II | | | | | | |
| | antigen-associated) | | | | | | |
| 24 | Cytochrome b-245, | Cyba | 1454268_a_at | 75 | NM_007806.1 | 76 | NP_031832.1 |
| | alpha polypeptide | | | | | | |
| 25 | N/A; transporter 1, | N/A; | 1416016_at | 77; 78; | AK166046.1; AW048052.1; | | |
| | ATP-binding | Tap1 | | 79 | ENSMUST00000041633.5 | | |
| | cassette, sub-family | (74%) | | | | | |
| | B (MDR/TAP) | | | | | | |
| | {POOR HIT 74%} | | | | | | |
| 26 | RIKEN cDNA | 0610037M15Rik | 1431008_at | 80 | XM_903697.2 | 81 | XP_908790.2 |
| | 0610037M15 gene | | | | | | |
| 27 | similar to | N/A; | 1451318_a_at | 82; 84; | NM_010747.1; | 83; 85; | NP_034877.1; |
| | Yamaguchi sarcoma | Lyn | | 86 | XM_973394.1; | 87 | XP_978488.1; |
| | viral (v-yes-1) | | | | XM_991890.1 | | XP_996984.1 |
| | oncogene homolog; | | | | | | |
| | Yamaguchi sarcoma | | | | | | |
| | viral (V-yes-1) | | | | | | |
| | oncogene homolog | | | | | | |
| 28 | N/A; interleukin 2 | N/A; | 1416296_at | 88; 89 | ENSMUST00000033664.5; | 90 | AAA39286.1 |
| | receptor, gamma | Il2rg | | | L20048.1 | | |
| | chain | | | | | | |
| 29 | !! [Ly86] | | 1422903_at | 91 | ENSMUST00000021860.3 | | |
| | Lymphocyte | | | | | | |
| | antigen 86 | | | | | | |
| 30 | membrane-spanning | Ms4a4b | 1423467_at | 92 | NM_021718.2 | 93 | NP_068364.1 |
| | 4-domains, | | | | | | |
| | subfamily A, | | | | | | |
| | member 4B | | | | | | |

Table 2 provides the top genes of Table 1A inducted by UC10 ranked by fold changes.

TABLE 2
Top genes induced by UC10 (ranked by fold changes)

| Probe Set ID | Gene Symbol | p-value | Fold change |
|---|---|---|---|
| 1425324_x_at | Igh-4 | 0.0024827 | 42.01 |
| 1425247_a_at | Igh-4 | 0.0026075 | 33.24 |
| 1427455_x_at | Igk-V8 | 0.0012493 | 31.51 |
| 1424305_at | Igj | 0.0037365 | 29.12 |
| 1427756_x_at | Igh-4 | 0.0037902 | 26.47 |
| 1452463_x_at | Igk-V8 | 0.0022802 | 23.03 |
| 1452417_x_at | 2010205A11Rik | 0.0018467 | 20.49 |
| 1427660_x_at | Igk-V8 | 0.0015737 | 20.02 |
| 1451632_a_at | Igh-6 | 0.0067362 | 20.02 |
| 1427351_s_at | Igh-6 | 0.00119 | 19.43 |
| 1430523_s_at | Igl-V1 | 0.0058301 | 16.77 |
| 1424931_s_at | Igl-V1 | 0.0060744 | 16.38 |
| 1427329_a_at | Igh-6 | 0.0039934 | 15.09 |
| 1427870_x_at | Igh-4 | 0.0041593 | 14.59 |
| 1423467_at | Ms4a4b | 0.0001104 | 12.83 |
| 1424825_a_at | Glycam1 | 0.0074547 | 11.35 |
| 1425738_at | LOC243469 | 0.0070091 | 10.97 |
| 1419426_s_at | Ccl21b /// Ccl21a /// Ccl21c | 0.0077637 | 9.96 |
| 1425871_a_at | LOC384413 /// LOC434038 | 0.0147353 | 9.93 |
| 1417851_at | Cxcl13 | 0.0004093 | 9.67 |
| 1426772_x_at | Tcrb-V13 | 0.0010696 | 9.47 |
| 1426113_x_at | Tcra | 0.0005445 | 9.26 |
| 1425226_x_at | Tcrb-V13 | 0.0011634 | 8.66 |
| 1448377_at | Slpi | 0.003984 | 8.46 |
| 1452205_x_at | Tcrb-V13 | 0.0020205 | 8.18 |
| 1426174_s_at | Ighg | 0.0095374 | 8.05 |
| 1417640_at | Cd79b | 0.00978 | 8.00 |
| 1452557_a_at | — | 0.0058116 | 7.93 |
| 1425854_x_at | Tcrb-V13 | 0.002944 | 7.58 |
| 1422828_at | Cd3d | 0.0018528 | 7.52 |

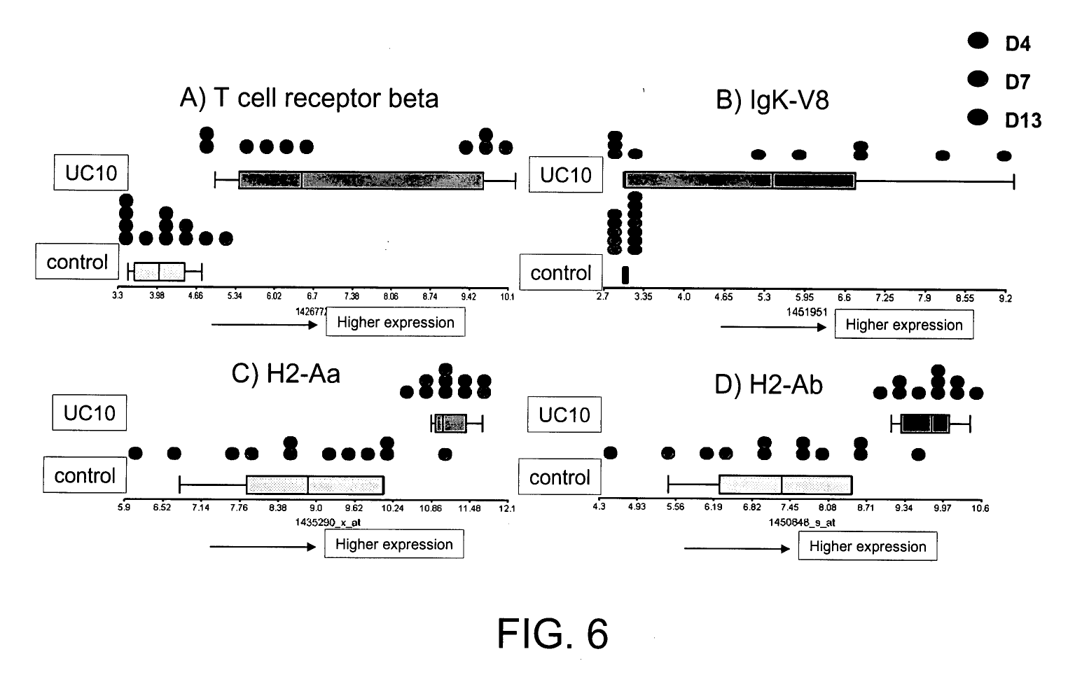

FIG. 6 illustrates the expression levels of T cell receptor beta, immunoglobulin, and class II MHC genes in the tumor RNA expression profile analysis. These genes were induced by UC10 treatment, suggesting the infiltration of T, B, and possibly antigen presenting cells as well in the tumor.

Ubd (FAT10/diubiquitin) gene was induced strongly by UC10 treatment. This gene may be a marker for the activation of anti-tumor immunity. Ubd gene is known to be induced by IFN-gamma and TNF-alpha, but its role in anti-immune response has not been described in the literature. (A. Canaan et al., Mol. Cell. Biol., July; 26(13):5180-9 (2006); E. Bates et al., Eur. J. Immunol., October; 27(10):2471-7 (1997))

### Example 2

### Identification of Biomarkers Using EMT-6 Mouse Tumor Model

The effect of UC10 vs. control (hamster IgG) was studied. Initially, tumor and blood total RNA were analyzed by qPCR. This was followed by RNA expression profile analysis using Affymetrix GeneChip.

### Animal Sample Collection:

EMT6 mouse mammary tumor was maintained in vitro. (Rockwell and Kallman, Radiat Res., February; 53(2):281-94 (1973)) EMT6 tumor cells were injected in the subcutaneous space of the right flank of Balb/c mice. Treatments were initiated when the subcutaneous tumor reached a median size between 100-200 mm³. 4.5 mg/kg of UC10 or Hamster IgG was injected intravenously once per week for three weeks (q7dx3 IV). At 24h after the third treatment of UC10, four mice were sacrificed. At seven days after the third treatment of hamster IgG, four mice were sacrificed.

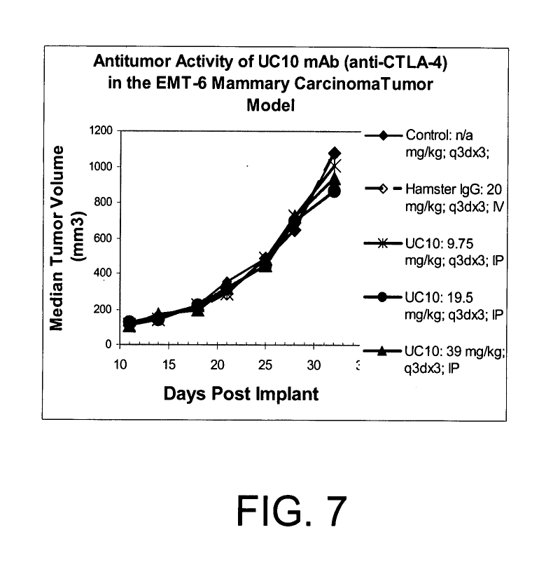

Separately, anti-tumor activity of UC10 was studied in EMT6 tumor cells. The results are provided in FIG. 7, wherein UC10 treatments resulted in no activity at doses as high as 39 mg/kg.

RNA Expression Analysis by Quantitative PCR (qPCR):

Tumor and blood samples were collected from these mice. Tumors were collected in RNAlater, while blood samples were collected in PAXgene tubes. Total RNA was extracted from the blood using PAXgene RNA extraction kit. Tumor RNA was extracted using Qiagene RNAeasy mini kit. The quality of isolated RNA was confirmed using Agilent 2100 nucleic acid analyzer detecting the peaks for 18S and 28S rRNA. cDNA was synthesized with a reverse transcription kit (Applied Biosystems) using random hexamers as primers.

qPCR was performed using the Taqman method using Applied Biosystems (AB)'s ‘Assay on Demand (AoD)’ premade assays. Some of the analysis was done using individual Taqman reagents in 96-well formats. Other assays were performed using Taqman Low Density Array (TLDA) microfluidic card system. All of these assays were run on the 7900HT sequence detection machine (AB).

### Tumor Result:

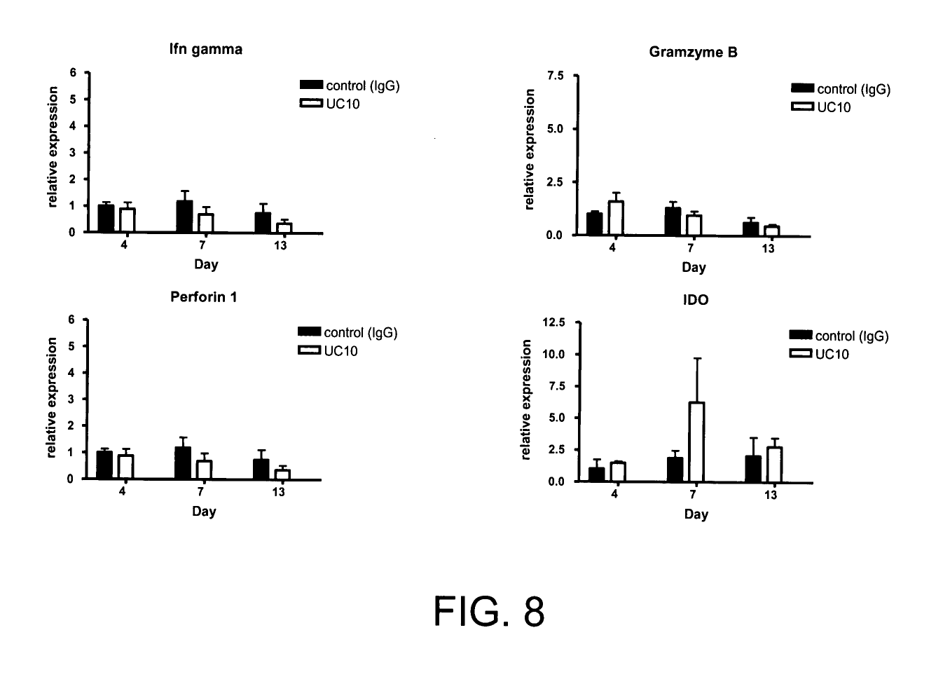

Tumor qPCR analysis was done for all the samples including the UC10 treated arm. FIG. 8 illustrates the EMT6 tumor RNA qPCR results. As shown in FIG. 8, EMT6 model does not respond to the UC10 treatment. In this model, UC10 did not induce the induction of interferon gamma, granzyme B, or perforin 1 genes suggesting there was no activation of the immune response in the tumor.

### Blood Result:

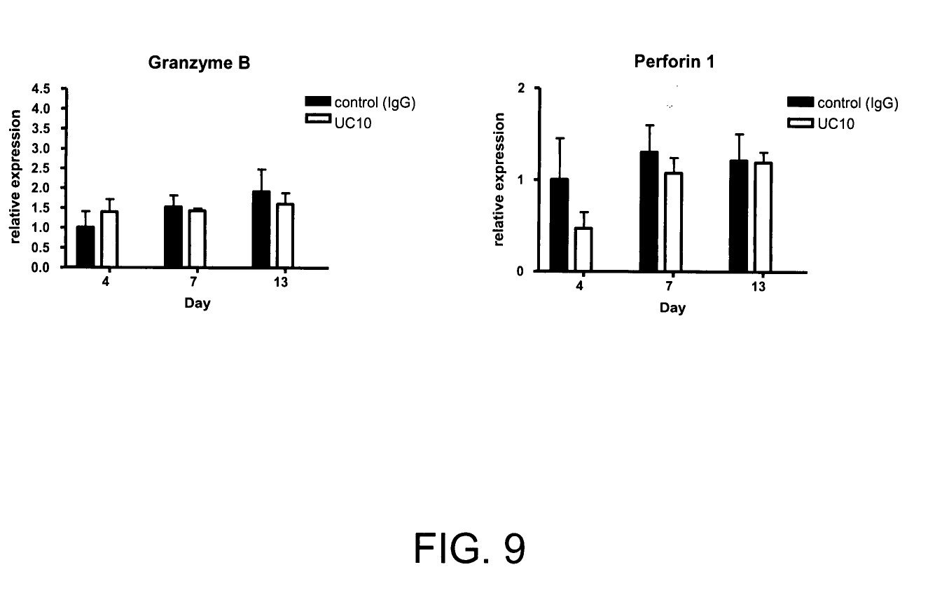

The same set of genes were analyzed in the blood. As illustrated in FIG. 9, UC10 did not induce granzyme B or perforin 1 gene expression in the blood of EMT6 model. This contrasted with the Sa1N model, again reflecting the lack of anti-tumor response in this model.

### Tumor RNA Expression Analysis:

The tumor cells were collected and tumor RNA extracted as described above. The Affymetrix GeneChip process and data analysis as described above. The blood analysis protocol was identical to the tumor analysis, except for the globin reduction pretreatment that was required since a pilot experiment indicated that mouse blood RNA generated a poor quality RNA expression data without the globin reduction process. Globin reduction was performed using the Ambion protocol following the manufacturer's manual.

UC10 effect was analyzed for one time point (1 day after the third treatment), as this was the only time the samples were collected. The analysis was done as a one-way ANOVA (essentially a t-test) using Partek software. None of the genes appeared to be changed significantly by UC10. Also, the genes with the lowest p-value for treatment effect appeared from random from various pathways. This reflects the efficacy results.

### Blood RNA Expression Analysis:

To further identify genes induced by UC10 in the blood, the same RNA samples were analyzed by RNA expression profiling using Affymetrix mouse A430_{—}2 GeneChip.

The RNA expression data was exported from Xpress using the RMA method with log₂ transformation. The entire data was loaded onto Partek software and the subsequent analysis was performed using this software. Initially, the genes were filtered to select for those with a reliable signal and inter sample variations, applying the following two criteria: (i) CV>10% (to remove the genes that did not have significant variation among different samples); and (ii) the maximal signal >6.6 (to select genes with a reliable expression level at least in one of the samples), which corresponds to the signal of 100 without log 2 transformation.

The top 29 genes (probes) based on the p-value are provided in Table 3A.

TABLE 3A
Top 29 genes induced by UC10 (ranked by p-values)

| | | | Fold | p- |
|---|---|---|---|---|
| Probe Set ID | Gene Symbol | Gene Title | change | value (treatment) |
| 1425295_at | Ear11 | eosinophil-associated, | 30.75 | 0.00030 |
| | | ribonuclease A family, | | |
| | | member 11 | | |
| 1419764_at | Chi3l3 | chitinase 3-like 3 | 46.74 | 0.00046 |
| 1455530_at | — | — | 0.37 | 0.00059 |
| 1422122_at | Fcer2a | Fc receptor, IgE, low affinity | 0.34 | 0.00098 |
| | | II, alpha polypeptide | | |
| 1416746_at | H2afx | H2A histone family, | 2.51 | 0.00156 |
| | | member X | | |
| 1425451_s_at | Chi3l3 /// | chitinase 3-like 3 /// | 20.60 | 0.00176 |
| | Chi3l4 | chitinase 3-like 4 | | |
| 1419549_at | Arg1 | arginase 1, liver | 24.05 | 0.00205 |
| 1449015_at | Retnla | resistin like alpha | 7.98 | 0.00239 |
| 1420249_s_at | Ccl6 | chemokine (C-C motif) | 2.94 | 0.00285 |
| | | ligand 6 | | |
| 1450430_at | Mrc1 | mannose receptor, C type 1 | 6.34 | 0.00558 |
| 1455106_a_at | Ckb | creatine kinase, brain | 3.63 | 0.00567 |
| 1419515_at | Fgd2 | FYVE, RhoGEF and PH | 3.00 | 0.00582 |
| | | domain containing 2 | | |
| 1417936_at | Ccl9 | chemokine (C-C motif) | 3.15 | 0.00628 |
| | | ligand 9 | | |
| 1448898_at | Ccl9 | chemokine (C-C motif) | 3.04 | 0.00783 |
| | | ligand 9 | | |
| 1418509_at | Cbr2 | carbonyl reductase 2 | 5.46 | 0.00817 |
| 1417346_at | Pycard | PYD and CARD domain | 2.01 | 0.00897 |
| | | containing | | |
| 1425469_a_at | — | — | 2.07 | 0.00900 |
| 1438009_at | Hist1h2ad | Histone 1, H2ae, mRNA | 3.20 | 0.00945 |
| | | (cDNA clone MGC: 90847 | | |
| | | IMAGE: 5713252) /// CDNA | | |
| | | clone MGC: 103288 | | |
| | | IMAGE: 5150365 | | |
| 1423756_s_at | Igfbp4 | insulin-like growth factor | 0.45 | 0.01371 |
| | | binding protein 4 | | |
| 1455332_x_at | Fcgr2b | Fc receptor, IgG, low | 2.80 | 0.01505 |
| | | affinity IIb | | |
| 1451941_a_at | Fcgr2b | Fc receptor, IgG, low | 3.88 | 0.01528 |
| | | affinity IIb | | |
| 1434437_x_at | Rrm2 | ribonucleotide reductase M2 | 3.78 | 0.01718 |
| 1435476_a_at | Fcgr2b | Fc receptor, IgG, low | 2.41 | 0.01721 |
| | | affinity IIb | | |
| 1448883_at | Lgmn | legumain | 2.15 | 0.01815 |
| 1435477_s_at | Fcgr2b | Fc receptor, IgG, low | 3.23 | 0.02094 |
| | | affinity IIb | | |
| 1460287_at | Timp2 | tissue inhibitor of | 3.14 | 0.02462 |
| | | metalloproteinase 2 | | |
| 1416108_a_at | Tmed3 | transmembrane emp24 | 1.87 | 0.02477 |
| | | domain containing 3 | | |
| 1416713_at | 2700055K07Rik | RIKEN cDNA 2700055K07 | 2.32 | 0.02703 |
| | | gene | | |
| 1430523_s_at | Igl-V1 | immunoglobulin lambda | 1.99 | 0.02782 |
| | | chain, variable 1 | | |

Table 3 provides the biomarkers of Table 3A.

TABLE 3
Biomarkers of Table 3A

| | | | | DNA | | | |
|---|---|---|---|---|---|---|---|
| | NCBI Gene | | | SEQ | | Protein | |
| | (LocusLink) | Gene | Probe Set | ID | DNA | SEQ ID | Protein |
| Entry | Title | Symbol | ID | NO: | Accession | NO: | Accession |
| 1 | Eosinophil- | Ear11 | 1425295_at | 94 | NM_053113.2 | 95 | NP_444343.2 |
| | associated, | | | | | | |
| | ribonuclease | | | | | | |
| | A family, | | | | | | |
| | member 11 | | | | | | |
| 2 | chitinase 3- | Chi313 | 1419764_at | 96 | XM_992616.1 | 97 | XP_997710.1 |
| | like 3 | | | | | | |
| 3 | !! uv90d11.x1 Soares | | 1455530_at | 98 | BE686052.1 | | |
| | mouse 3NbMS Mus | | | | | | |
| | musculus cDNA clone | | | | | | |
| | IMAGE: 3414453 3′, | | | | | | |
| | mRNA sequence | | | | | | |
| 4 | Fc receptor, | Fcer2a | 1422122_at | 99 | NM_013517.1 | 100 | NP_038545.1 |
| | IgE, low | | | | | | |
| | affinity II, | | | | | | |
| | alpha | | | | | | |
| | polypeptide | | | | | | |
| 5 | H2A | H2afx | 1416746_at | 101 | NM_010436.2 | 102 | NP_034566.1 |
| | histone | | | | | | |
| | family, | | | | | | |
| | member X | | | | | | |
| 6 | chitinase 3- | Chi313; | 1425451_s_at | 96; | XM_992616.1; | 97; 104; | XP_997710.1; |
| | like 3; | Chi314 | | 103; | NM_009892.1; | 106 | NP_034022.1; |
| | chitinase 3- | | | 105 | NM_145126.1 | | NP_660108.1 |
| | like 4 | | | | | | |
| 7 | N/A; | N/A; | 1419549_at | 107; | BC050005.1; | 108; 111 | AAH50005.2; |
| | arginase 1, | Arg1 | | 109; | ENSMUST00000020161.5; | | AAA98611.1 |
| | liver | (65%) | | 110 | U51805.1 | | |
| | {POOR | | | | | | |
| | HIT 65%} | | | | | | |
| 8 | resistin like | Retnla | 1449015_at | 112 | NM_020509.3 | 113 | NP_065255.2 |
| | alpha | | | | | | |
| 9 | chemokine | Cc16 | 1420249_s_at | 114 | NM_009139.3 | 115 | NP_033165.1 |
| | (C-C motif) | | | | | | |
| | ligand 6 | | | | | | |
| 10 | Mannose | Mrc1 | 1450430_at | 116 | NM_008625.1 | 117 | NP_032651.1 |
| | receptor, C | | | | | | |
| | type 1 | | | | | | |
| 11 | N/A; | N/A; | 1455106_a_at | 118; | AK165779.1; | 122 | NP_067248.1 |
| | Creatine | Ckb | | 119; | BG967663.1; | | |
| | kinase, | | | 120; | ENSMUST00000001304.5; | | |
| | brain | | | 121 | NM_021273.3 | | |
| 12 | !! [Fgd2] | | 1419515_at | 123 | ENSMUST00000024810.4 | | |
| | FYVE, | | | | | | |
| | RhoGEF | | | | | | |
| | and PH | | | | | | |
| | domain | | | | | | |
| | containing 2 | | | | | | |
| 13 | Chemokine | Ccl9 | 1417936_at | 124; | AF128196.1; | 125; 127 | AAF22537.1; |
| | (C-C motif) | | | 126 | NM_011338.2 | | NP_035468.1 |
| | ligand 9 | | | | | | |
| 14 | Chemokine | Ccl9 | 1448898_at | 126 | NM_011338.2 | 127 | NP_035468.1 |
| | (C-C motif) | | | | | | |
| | ligand 9 | | | | | | |
| 15 | Carbonyl | Cbr2 | 1418509_at | 128 | NM_007621.1 | 129 | NP_031647.1 |
| | reductase 2 | | | | | | |
| 16 | PYD and | Pycard | 1417346_at | 130 | NM_023258.3 | 131 | NP_075747.2 |
| | CARD | | | | | | |
| | domain | | | | | | |
| | containing | | | | | | |
| 17 | !! hypothetical protein | | 1425469_a_at | 132; | AK085738.1; | 134 | AAH03855.1 |
| | LOC624610\|\| | | | 133 | BC003855.1 | | |
| | hypothetical protein | | | | | | |
| | LOC675730 \|\| similar | | | | | | |
| | to IG KAPPA CHAIN | | | | | | |
| | V-V REGION L6 | | | | | | |
| | PRECURSOR | | | | | | |
| 18 | Similar to | MGC73635 | 1438009_at | 135; | XM_978296.1; | 136; 138 | XP_983390.1; |
| | histone 2a | (*) | | 137 | XM_978341.1 | | XP_983435.1 |
| 19 | Insulin-like | Igfbp4 | 1423756_s_at | 139 | NM_010517.3 | 140 | NP_034647.1 |
| | growth | | | | | | |
| | factor | | | | | | |
| | binding | | | | | | |
| | protein 4 | | | | | | |
| 20 | N/A; Fc | N/A; | 1455332_x_at | 141; | BM224327.2; | 143 | NP_034317.1 |
| | receptor, | Fcgr2b | | 142 | NM_010187.2 | | |
| | IgG, low | | | | | | |
| | affinity IIb | | | | | | |
| 21 | Fc receptor, | Fcgr2b | 1451941_a_at | 142 | NM_010187.2 | 143 | NP_034317.1 |
| | IgG, low | | | | | | |
| | affinity IIb | | | | | | |
| 22 | ribonucleotide | Rrm2 | 1434437_x_at | 144 | NM_009104.1 | 145 | NP_033130.1 |
| | reductase | | | | | | |
| | M2 | | | | | | |
| 23 | Fc receptor, | Fcgr2b | 1435476_a_at | 142 | NM_010187.2 | 143 | NP_034317.1 |
| | IgG, low | | | | | | |
| | affinity IIb | | | | | | |
| 24 | !! [Lgmn] | | 1448883_at | 146 | ENSMUST00000021607.5 | | |
| | Legumain | | | | | | |
| 25 | Fc receptor, | Fcgr2b | 1435477_s_at | 142 | NM_010187.2 | 143 | NP_034317.1 |
| | IgG, low | | | | | | |
| | affinity IIb | | | | | | |
| 26 | Tissue | Timp2 | 1460287_at | 147 | NM_011594.3 | 148 | NP_035724.2 |
| | inhibitor of | | | | | | |
| | metalloproteinase 2 | | | | | | |
| 27 | Transmembrane | Tmed3 | 1416108_a_at | 149 | NM_025360.1 | 150 | NP_079636.1 |
| | emp24 | | | | | | |
| | domain | | | | | | |
| | containing 3 | | | | | | |
| 28 | RIKEN | 2700055K07Rik | 1416713_at | 151 | NM_026481.2 | 152 | NP_080757.1 |
| | cDNA | | | | | | |
| | 2700055K07 | | | | | | |
| | gene | | | | | | |
| 29 | immunoglobulin | Igl-V1 | 1430523_s_at | 153; | AK008094.1; | 154; 156 | BAB25455.1; |
| | lambda | | | 155; | AK008145.1; | | BAB25493.1 |
| | chain, | | | 157 | AY170495.1 | | |
| | variable 1 | | | | | | |

The highest induction was seen with genes associated with Th2 response or alternative activation of macrophage. Both of these pathways can be anti-inflammatory, and may suppress anti-tumor immunity. It should be also noted that none of these patterns was observed in the tumor gene expression from the same mice.

Earl1 is one of the mouse versions of eosinophil cationic protein (ECP), which is one of the mediators of the Th2 response.

Tables 3A and 3 include genes associated with alternative activation of macrophages (Ym1, arginase 1 and Fizz 1). Arginasel, Chi313 (Ym1), Retnla (resistin-like alpha/Fizz1) have all been reported to be markers of alternatively activated macrophages in mice. (Nair et al., Immunol Lett., January 22; 85(2):173-80 (2003)) Mrc1 may also be involved in the type-2 activation of monocyte derived DCs, which can be anti-inflammatory. (Chieppa et al., J. Immunol., November 1; 171(9):4552-60 (2003))

Activation of creatine kinase has been observed during monocyte to macrophage development. (Loike et al., J Exp Med., March 1; 159(3):746-57 (1984))

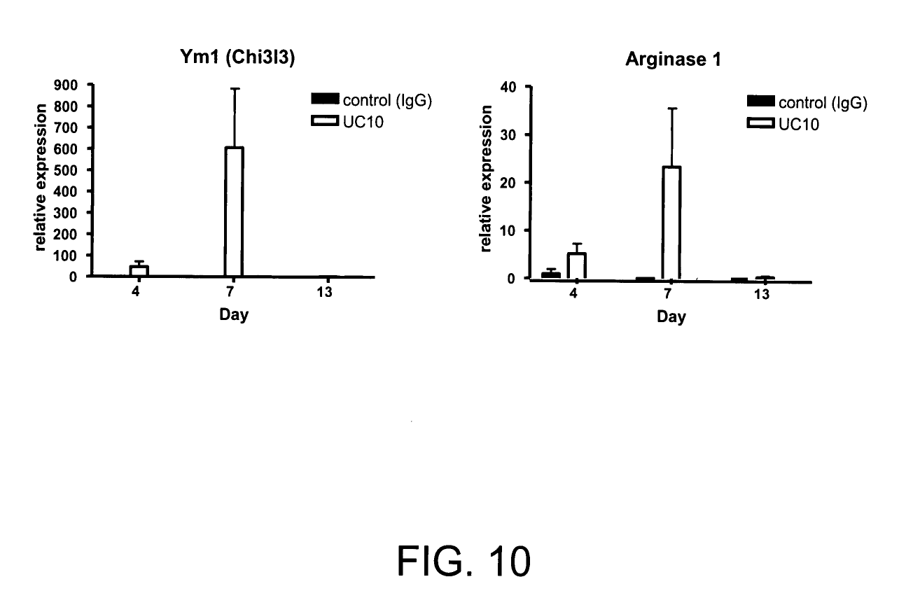

The same blood RNA samples analyzed in FIG. 9 were analyzed for the expression of Ym1 and ariginase 1 and the results are provided in FIG. 10. The induction of these genes by UC10 was reproduced, and the expression level was extremely high at day 7. This high expression of Ym1 and arginase 1 mostly disappeared by day 13.

### Example 3

### Production of Antibodies Against the Biomarkers

Antibodies against the biomarkers can be prepared by a variety of methods. For example, cells expressing a biomarker polypeptide can be administered to an animal to induce the production of sera containing polyclonal antibodies directed to the expressed polypeptides. In one aspect, the biomarker protein is prepared and isolated or otherwise purified to render it substantially free of natural contaminants, using techniques commonly practiced in the art. Such a preparation is then introduced into an animal in order to produce polyclonal antisera of greater specific activity for the expressed and isolated polypeptide.

In one aspect, the antibodies of the invention are monoclonal antibodies (or protein binding fragments thereof). Cells expressing the biomarker polypeptide can be cultured in any suitable tissue culture medium, however, it is preferable to culture cells in Earle's modified Eagle's medium supplemented to contain 10% fetal bovine serum (inactivated at about 56° C.), and supplemented to contain about 10 g/l nonessential amino acids, about 1.00 U/ml penicillin, and about 100 μg/ml streptomycin.

The splenocytes of immunized (and boosted) mice can be extracted and fused with a suitable myeloma cell line. Any suitable myeloma cell line can be employed in accordance with the invention, however, it is preferable to employ the parent myeloma cell line (SP2/0), available from the ATCC (Manassas, Va.). After fusion, the resulting hybridoma cells are selectively maintained in HAT medium, and then cloned by limiting dilution as described by Wands et al. (1981, Gastroenterology, 80:225-232). The hybridoma cells obtained through such a selection are then assayed to identify those cell clones that secrete antibodies capable of binding to the polypeptide immunogen, or a portion thereof.

Alternatively, additional antibodies capable of binding to the biomarker polypeptide can be produced in a two-step procedure using anti-idiotypic antibodies. Such a method makes use of the fact that antibodies are themselves antigens and, therefore, it is possible to obtain an antibody that binds to a second antibody. In accordance with this method, protein specific antibodies can be used to immunize an animal, preferably a mouse. The splenocytes of such an immunized animal are then used to produce hybridoma cells, and the hybridoma cells are screened to identify clones that produce an antibody whose ability to bind to the protein-specific antibody can be blocked by the polypeptide. Such antibodies comprise anti-idiotypic antibodies to the protein-specific antibody and can be used to immunize an animal to induce the formation of further protein-specific antibodies.

### Example 4

### Immunofluorescence Assays

The following immunofluorescence protocol may be used, for example, to verify CTLA-4 biomarker protein expression on cells or, for example, to check for the presence of one or more antibodies that bind CTLA-4 biomarkers expressed on the surface of cells. Briefly, Lab-Tek II chamber slides are coated overnight at 4° C. with 10 micrograms/milliliter (μg/ml) of bovine collagen Type II in DPBS containing calcium and magnesium (DPBS++). The slides are then washed twice with cold DPBS++ and seeded with 8000 CHO—CCR5 or CHO pC4 transfected cells in a total volume of 125 μl and incubated at 37° C. in the presence of 95% oxygen/5% carbon dioxide.

The culture medium is gently removed by aspiration and the adherent cells are washed twice with DPBS++ at ambient temperature. The slides are blocked with DPBS++ containing 0.2% BSA (blocker) at 0-4° C. for one hour. The blocking solution is gently removed by aspiration, and 125 μl of antibody containing solution (an antibody containing solution may be, for example, a hybridoma culture supernatant which is usually used undiluted, or serum/plasma which is usually diluted, e.g., a dilution of about 1/100 dilution). The slides are incubated for 1 hour at 0-4° C. Antibody solutions are then gently removed by aspiration and the cells are washed five times with 400 μl of ice cold blocking solution. Next, 125 μl of 1 μg/ml rhodamine labeled secondary antibody (e.g., anti-human IgG) in blocker solution is added to the cells. Again, cells are incubated for 1 hour at 0-4° C.

The secondary antibody solution is then gently removed by aspiration and the cells are washed three times with 400 μl of ice cold blocking solution, and five times with cold DPBS++. The cells are then fixed with 125 μl of 3.7% formaldehyde in DPBS++ for 15 minutes at ambient temperature. Thereafter, the cells are washed five times with 400 μl of DPBS++ at ambient temperature. Finally, the cells are mounted in 50% aqueous glycerol and viewed in a fluorescence microscope using rhodamine filters.

What is claimed is:

1. A method for predicting the likelihood a mammal will respond therapeutically to a method of treating cancer comprising administering an CTLA-4 antagonist, wherein the method comprises:
(a) measuring in the mammal the level of at least one biomarker selected from the biomarkers of Table 1 and Table 3;
(b) exposing a biological sample from said mammal to the CTLA-4 antagonist;
(c) following the exposing of step (b), measuring in said biological sample the level of the at least one biomarker,
wherein an increase in the level of the at least one biomarker measured in step (c) compared to the level of the at least one biomarker measured in step (a),
indicates an increased likelihood that the mammal will respond therapeutically to said method of treating cancer when said at least one biomarker is from Table 1, and
indicates an increased likelihood that the mammal will not respond therapeutically to said method of treating cancer when said at least one biomarker is from Table 3.

2. The method of claim 1 wherein said at least one biomarker further comprises at least one additional biomarker.

3. The method of claim 1 wherein said biological sample is a tumor or blood sample.
